(19) United States
(12) Patent Application Publication
(10) Pub. No.: US 2023/0174953 A1
(43) Pub. Date: Jun. 8, 2023

(54) DEFECTIVE INTERFERING VIRAL GENOMES

(71) Applicant: INSTITUT PASTEUR, PARIS CEDEX 15 (FR)

(72) Inventors: MARCO VIGNUZZI, Paris (FR); BJORN MEYER, Paris (FR); VERONICA REZELJ, Paris (FR); LAURA LEVI, Paris (FR); VERONIKA BERNHAUEROVA, Paris (FR); THOMAS VALLET, Paris (FR); TANGUY PIEPLU, Paris (FR); DJOSHKUN SHENGJULER, Paris (FR); STEPHANIE BEAUCOURT, Paris (FR); HERVE BLANC, Paris (FR); NATHALIE PARDIGON, Paris (FR); GIOVANNA BARBA-SPAETH, Paris (FR); MARIA-CARLA SALEH, Paris (FR)

(21) Appl. No.: 17/907,424

(22) PCT Filed: Mar. 26, 2021
(86) PCT No.: PCT/IB2021/000231

§ 371 Date: Sep. 27, 2022

Related U.S. Application Data
(60) Provisional application No. 63/000,998, filed on Mar. 27, 2020.

(51) Int. Cl. C12N 7/00

(52) U.S. Cl. CPC C12N 7/00; C12N 2770/20021; C12N 2770/20034; C12N 2770/32321; C12N 2770/32334; C12N 2770/32721; C12N 2770/32734; C12N 2770/36121; C12N 2770/36134

(57) ABSTRACT

Method for producing a defective interfering viral genome (DVG), defective interfering particles comprising the DVG, and methods and uses thereof.

A part of the invention was made with United States Government Support under Agreement No.: HR0011-17-2-0023 awarded by the Defense Advanced Research Projects Agency (DARPA). The United States Government has certain rights in the invention.

### INTRODUCTION

This invention was made with government support under DARPA agreement number HR00111720023 awarded by the Defense Advanced Research Projects Agency (DARPA).Viruses that possess an RNA genome have polymerases with an intrinsically high error rate. As a consequence of this error prone replication process, these viruses do not only generate full-length viral genomes or genomes with point mutations, but furthermore generate defective genomes. Various types of defective genomes have been described, which include truncations, insertions, deletions, mosaic or rearranged genomes and copyback/snapbacks. While the majority of defective genomes are thought to be dead-end side-products of RNA virus replication, a subset of these defective viral genomes (DVGs) interfere with the generation of infectious progeny virus particles to high numbers.

Along with high mutation rates, recombination is another main driving force of RNA virus evolution. Non-homologous recombination can give rise to truncated and/or rearranged viral genomes that constitute defective viral genomes (DVGs). First described in 1954 by Von Magnus in influenza A virus, DVGs have since been described in all viral families. Because they lack part of their genome or its encoded functions, DVGs must co-infect cells with their parental virus, in order to take advantage of the proteins encoded by the full-length virus. Hence, DVGs that hijack the replication machinery or use proteins encoded by the parental virus, may compete with wild type virus for resources, which can result in inhibition of the parental virus. These types of DVGs are often referred to as defective interfering particles (DIPs). Furthermore, many DVGs have a strong, immunostimulatory potential both in mammals and invertebrates. DVGs were identified in sera from patients suffering from acute dengue virus infection, but their pathophysiological role remains unknown. In humans, their presence correlates with milder disease and better outcome in influenza virus and respiratory syncytial virus infections. All these reasons justify renewed interest in using DVGs as antiviral therapy.

While DVGs have been described to exist for most viruses, DVGs were not considered to be relevant or predominant in in many virus families. Generally, only a few DVGs have been described for any given virus. These previous reports relied on more classic methods of isolation, such as PCR amplification, that select for only one or two DVGs and bias towards the shortest or most abundant DVGs. These DVGs are not necessarily the best candidates in terms of ability to compete with wild type virus. A need thus exists for reproducible and rational methods to identify and generate the best DVG candidates to compete and interfere with wild type virus infection, that could be applied to any virus of interest, especially human pathogens. The inventions disclosed herein meet these and other needs.

Our combined experimental evolution and computational approach identifies thousands of different DVGs for any virus. First, our experimental approach generates all possible DVGs in a virus population. Expectedly, our list includes DVGs that resemble those identified by more classic methods. Second, our computational analysis allows us to predict which DVGs would work best as defective interfering particles, using temporal frequencies estimated from the sequence data. Using this combined approach, we have generated a list of top candidates for a wide range of viruses belonging to different virus families, of which up to 90% have confirmed capacity to interfere with wild type virus replication.

### DESCRIPTION

This disclosure provides methods of generating DVGs and also provides new DVGs generated by the methods. DVGs generated from chikungunya virus (CHIKV), Zika virus (ZIKV), enterovirus 71 (EV71), West Nile virus (WNV), yellow fever virus (YFV) and rhinovirus (RV), and coronavirus (CV) (for example, the recently identified novel coronavirus named SARS-CoV-2, 2019-nCov or COVID-19) are provided.

The inventors have identified DVGs using next-generation sequencing methods followed by computational analysis, hotspot analysis identified a variety of DVGs that were deletion DVGs (internal deletions in the viral genomes of CHIKV, EV71, ZIKV as well as RV). Subsequently, these DVGs were genetically engineered and tested for their ability to reduce infectious viral titers of CHIKV, EV71, ZIKV as well as RV. Successful DVG candidates have the potential to be used as therapeutic interfering particles (TIPs), which could be used as a new strategy to treat and limit the severity of virus infections. Furthermore, DVGs for WNV and YFV were also identified. Based on success with other viruses, these DVGs have potential to be used as TIPs.

In a first aspect, a method for producing a defective interfering viral genome (DVG) is provided. The method comprises providing a first set of at least three replicate in vitro cell cultures, comprising a solid support and cells infected with a reference infectious virus at a high multiplicity of infection (MOI); providing a second set of at least three replicate in vitro cell cultures, comprising a solid support and cells infected with the reference infectious virus at a low multiplicity of infection (MOI); culturing the first and second sets of replicate in vitro cell cultures for at least 5 passages under normal growth conditions; collecting a plurality of DVG candidates from the medium of the first and second sets of in vitro replicate cell cultures; deep sequencing the collected DVG candidates; and selecting a subset of DVGs from the plurality of DVG candidates.

In some embodiments the method further comprises providing a third set of at least three replicate in vitro cell cultures, comprising a solid support and cells infected with a reference infectious virus at a high multiplicity of infection (MOI); and/or providing a fourth set of at least three replicate in vitro cell cultures, comprising a solid support and cells infected with the reference infectious virus at a low multiplicity of infection (MOI); and culturing the third and/or fourth sets of replicate in vitro cell cultures for at least 5 passages under mutagenic conditions ; or using a reference infectious virus with a mutator phenotype to perform said passages.

In some embodiments selecting a DVG from the plurality of DVG candidates comprises: comparing the sequences of the DVG candidates with the sequence of the genome of the reference infectious virus; identifying at least one DVG candidate having a genome comprising at least one splicing event where the splicing event is a deletion or a rearrangement; determining the relative frequency of at least one DVG candidate having a genome with at least one splicing event among the population of sequenced DVG candidate genomes; and, selecting at least one DVG for which: a) at least one splicing event is more abundant at high MOI cultures than at low MOI cultures; and/or b) at least one splicing event appears in at least 2, 3, 4, 5, 6, 7, 8 or 9 different cell lines; and/or, c) at least one splicing event is found in at least 3 of at least 12, 24 or 36 independent replicates, after at least 5, 10, 15 or 20 passages; and/or, d) at least one splicing event is a deletion event and the nucleotide size of the deletion is at least 40, 42, 50, 100, 150, 200, 400, 600 or 1000 nucleotides; and/or, e) an open reading frame in the DVG is maintained; and/or, f) the structural domains and functions necessary for viral replication, are maintained in the DVG; and/or g) the DVG comprises at least one mutation that reduces the self-replicative capacity of the reference infectious virus.

In some embodiments the DVG is characterized by an in vitro inhibitory activity of at least 50%, 60%, 70%, 80% or 90% against the reference infectious virus. In some embodiments the reference infectious virus is a chikungunya virus (CHIKV), Zika virus (ZIKV), enterovirus 71 (EV71), rhinovirus (RV), yellow fever virus (YFV) or West Nile virus (WNV). In some embodiments the reference infectious virus is selected from the group consisting of: CHIKV Indian Ocean lineage (GenBank accession no. AM258994), CHIKV Caribbean strain (GenBank accession no. LN898104.1), ZIKV African strain MR766 (KY989511.1), EV71 Sep006 (GenBank: KX197462.1), RV-A01a (NC_038311.1), RV-A16 (L24917.1), RV-B14 (L05355.1), RV-C15 (GU219984.1), West Nile virus Israel 1998 and YFV strain, Yellow Fever Virus Asibi (YF-Asibi) strain and Yellow Fever Virus vaccine (YF-17D) strain. In some embodiments the reference infectious virus is SARS-CoV-2 (Zhu N et al., N Engl J Med., 2020 Jan 24). In some embodiments the cells are a mammalian cell line selected from the group consisting of: a cell line derived from African green monkey kidney cells (optionally Vero, optionally Vero-E6 cell line), a cell line derived from human muscle (optionally RD cell line), a cell line derived from baby hamster kidneys (optionally BHK cell line), a cell line derived from human embryonic kidney cells (optionally HEK293T cell line) a cell line derived from liver carcinoma cells (optionally Huh7 cell line), and a cell line derived from cervical adenocarcinoma (optionally H1-HeLa, optionally HeLa-E8 cell line). In some embodiments the cells are mosquito a cell line selected from the group consisting of: a cell line derived from Aedes aegypti (optionally Aag2 cell line), a cell derived from Aedes albopictus (optionally C6/36 cell line) and a cell line derived from Aedes albopictus (optionally U4.4 cell line). In some embodiments the low MOI for infection is from 0.001 to 0.1 PFU/cell, in particular from 0.01 to 0.1 PFU/cell. In some embodiments the high MOI for infection is from 1 to 100 PFU/cell.

Also provided is a DVG produced by the method of this invention.

Also provided is a DVG comprising or consisting of a nucleotide sequence selected from the group consisting of: SEQ ID NO: 1 (DG1 : EV71 DVG 293-390), SEQ ID NO: 2 (DG2 : EV71 DVG 294-404), SEQ ID NO: 3 (DG3 : EV71 DVG 1746-2895), SEQ ID NO: 4 (DG4 : EV71 DVG 1752-2894), SEQ ID NO: 5 (DG5 : EV71 DVG 1752-2896), SEQ ID NO: 6 (DG6 : EV71 DVG 1880-6487), SEQ ID NO: 7 (DG7 : EV71 DVG 1880-6488), SEQ ID NO: 8 (DG8 : EV71 DVG 3513-6516), SEQ ID NO: 9 (DG9 : EV71 DVG 5475-5634), SEQ ID NO: 10 (DG10: EV71 DVG 5610-6876), SEQ ID NO: 11 (DG11 : EV71 DVG 5610-6877), SEQ ID NO: 12 (DG12: EV71 DVG 5746-5821), SEQ ID NO: 13 (DG13 : EV71 DVG 6322-6356), SEQ ID NO: 14 (DG14: EV71 DVG 6322-6358), SEQ ID NO: 15 (DG15: EV71 DVG 6728-6779), SEQ ID NO: 16 (DG16: EV71 DVG 6966-7012), SEQ ID NO: 17 (DG17: EV71 DVG 6966-7014), SEQ ID NO: 18 (DG18: EV71 DVG 7098-7122), SEQ ID NO: 19 (DG19: EV71 DVG 7165-7200), SEQ ID NO: 20 (DG20: EV71 DVG 7165-7201), SEQ ID NO: 21 (DG21: EV71 DVG 7238-7292), SEQ ID NO: 22 (ZIKV DVG-A), SEQ ID NO: 23 (ZIKV DVG-B), SEQ ID NO: 24 (ZIKV DVG-C), SEQ ID NO: 25 (ZIKV DVG-D), SEQ ID NO: 26 (ZIKV DVG-E), SEQ ID NO: 27 (ZIKV DVG-F), SEQ ID NO: 28 (ZIKV DVG-G), SEQ ID NO: 29 (ZIKV DVG-H), SEQ ID NO: 30 (ZIKV DVG-I), SEQ ID NO: 31 (ZIKV DVG-J), SEQ ID NO: 32 (ZIKV DVG-K), SEQ ID NO: 33 (ZIKV DVG-L), SEQ ID NO: 34 (CHIKV DVG-IV1), SEQ ID NO: 35 (CHIKV DVG-IV2), SEQ ID NO: 36 (CHIKV DVG-IV3), SEQ ID NO: 37 (CHIKV DVG-IV4), SEQ ID NO: 38 (CHIKV DVG-IH1), SEQ ID NO: 39 (CHIKV DVG-IU1), SEQ ID NO: 40 (CHIKV DVG-CV1), SEQ ID NO: 41 (CHIKV DVG-CV2), SEQ ID NO: 42 (CHIKV DVG-CV3), SEQ ID NO: 43 (CHIKV DVG-CV4), SEQ ID NO: 44 (CHIKV DVG-CH1), SEQ ID NO: 45 (CHIKV DVG-CH2), SEQ ID NO: 46 (CHIKV DVG-CH3), SEQ ID NO: 47 (CHIKV DVG-CM1), SEQ ID NO: 48 (CHIKV DVG-CU1), SEQ ID NO: 49 (CHIKV DVG-CU2), SEQ ID NO: 50 (CHIKV DVG-CA1), SEQ ID NO: 51 (CHIKV DVG-CA2), SEQ ID NO: 52 (CHIKV DVG-CA3), SEQ ID NO: 53 (CHIKV DVG-CA4), SEQ ID NO: 55 (RV DVG A01a-TIP-01), SEQ ID NO: 56 (RV DVG A01a-TIP-02), SEQ ID NO: 57 (RV DVG A01a-TIP-03), SEQ ID NO: 58 (RV DVG A01a-TIP-04), SEQ ID NO: 59 (RV DVG A01a-TIP-05), SEQ ID NO: 60 (RV DVG A01a-TIP-06), SEQ ID NO: 62 (RV DVG A16-TIP-01), SEQ ID NO: 63 (RV DVG A16-TIP-02), SEQ ID NO: 64 (RV DVG A16-TIP-03), SEQ ID NO: 65 (RV DVG A16-TIP-04), SEQ ID NO: 66 (RV DVG A16-TIP-05), SEQ ID NO: 89 (RV DVG C15-TIP-01), SEQ ID NO: 90 (RV DVG C15-TIP-02), SEQ ID NO: 91 (RV DVG C15-TIP-03), SEQ ID NO: 92 (RV DVG C15-TIP-04), SEQ ID NO: 68 (RV DVG B14-TIP-01), SEQ ID NO: 69 (RV DVG B14-TIP-02), SEQ ID NO: 70 (RV DVG B14-TIP-03), SEQ ID NO: 71 (RV DVG B14-TIP-04), SEQ ID NO: 72 (RV DVG B14-TIP-05), SEQ ID NO: 73 (RV DVG B14-TIP-06), SEQ ID NO: 74 (RV DVG B14-TIP-07), SEQ ID NO: 75 (RV DVG B14-TIP-08), SEQ ID NO: 76 (RV DVG B14-TIP-09), SEQ ID NO: 77 (RV DVG B14-TIP-10), SEQ ID NO: 78 (RV DVG B14-TIP-11), SEQ ID NO: 79 (RV DVG B14-TIP-12), SEQ ID NO: 80 (RV DVG B14-TIP-13), SEQ ID NO: 81 (RV DVG B14-TIP-14), SEQ ID NO: 82 (RV DVG B14-TIP-15), SEQ ID NO: 83 (RV DVG B14-TIP-16), SEQ ID NO: 84 (RV DVG B14-TIP-17), SEQ ID NO: 85 (RV DVG B14-TIP-18), SEQ ID NO: 86 (RV DVG B14-TIP-19), SEQ ID NO: 87 (RV DVG B14-TIP-20), SEQ ID NO:93 (YFV DVG-A), SEQ ID NO: 94 (YFV DVG-B), SEQ ID NO: 95 (YFV DVG-C), SEQ ID NO: 96 (YFV DVG-D), SEQ ID NO: 97 (YFV DVG-E), SEQ ID NO: 98 (YFV DVG-F), SEQ ID NO: 99 (YFV DVG-G), SEQ ID NO: 100 (YFV DVG-H), SEQ ID NO: 101 (YFV DVG-I), SEQ ID NO: 102 (YFV DVG-J), SEQ ID NO: 103 (YFV DVG-K), SEQ ID NO: 104 (YFV DVG-L), SEQ ID NO:105 (WNV DVG-1), SEQ ID NO: 106 (WNV DVG-2), SEQ ID NO: 107 (WNV DVG-3), SEQ ID NO: 108 (WNV DVG-4), SEQ ID NO: 109 (WNV DVG-5), SEQ ID NO: 110 (WNV DVG-6), SEQ ID NO: 111 (SARS-CoV-2 DVG_1), SEQ ID NO: 112 (SARS-CoV-2 DVG_2) and SEQ ID NO: 113 (SARS-CoV-2 DVG_3), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

Also provided is a defective interfering particle comprising a DVG of this invention.

Also provided are methods of treating a viral infection in a subject, comprising administering an efficient therapeutic amount of at least one DVG or defective interfering particle according to this invention.

In some embodiments the DVG is administered as RNA, naked RNA or RNA formulated with appropriate carriers such as nanoparticles or lipids.

In some embodiments the DVG is administered as a packaged RNA, as a virus-like particle (VLP) or other packaging carrier.

In some embodiments the DVG is transcribed from a DNA under the control of a host-appropriate transcriptional promoter.

In some embodiments the at least one DVG is a defective interfering CHIKV genome. In some embodiments the at least one defective interfering CHIKV genome comprises or consists of a nucleotide sequence selected from the group consisting of: SEQ ID NO: 34 (CHIKV DVG-IV1), SEQ ID NO: 35 (CHIKV DVG-IV2), SEQ ID NO: 36 (CHIKV DVG-IV3), SEQ ID NO: 37 (CHIKV DVG-IV4), SEQ ID NO: 38 (CHIKV DVG-IH1), SEQ ID NO: 39 (CHIKV DVG-IU1), SEQ ID NO: 40 (CHIKV DVG-CV1), SEQ ID NO: 41 (CHIKV DVG-CV2), SEQ ID NO: 42 (CHIKV DVG-CV3), SEQ ID NO: 43 (CHIKV DVG-CV4), SEQ ID NO: 44 (CHIKV DVG-CH1), SEQ ID NO: 45 (CHIKV DVG-CH2), SEQ ID NO: 46 (CHIKV DVG-CH3), SEQ ID NO: 47 (CHIKV DVG-CM1), SEQ ID NO: 48 (CHIKV DVG-CU1), SEQ ID NO: 49 (CHIKV DVG-CU2), SEQ ID NO: 50 (CHIKV DVG-CA1), SEQ ID NO: 51 (CHIKV DVG-CA2), SEQ ID NO: 52 (CHIKV DVG-CA3) and SEQ ID NO: 53 (CHIKV DVG-CA4), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

In some embodiments the at least one defective interfering genome is a defective interfering ZIKV genome. In some embodiments the at least one interfering ZIKV genome comprises or consists of a nucleotide sequence selected from the group consisting of: SEQ ID NO: 22 (ZIKV DVG-A), SEQ ID NO: 23 (ZIKV DVG-B), SEQ ID NO: 24 (ZIKV DVG-C), SEQ ID NO: 25 (ZIKV DVG-D), SEQ ID NO: 26 (ZIKV DVG-E), SEQ ID NO: 27 (ZIKV DVG-F), SEQ ID NO: 28 (ZIKV DVG-G), SEQ ID NO: 29 (ZIKV DVG-H), SEQ ID NO: 30 (ZIKV DVG-I), SEQ ID NO: 31 (ZIKV DVG-J), SEQ ID NO: 32 (ZIKV DVG-K) and SEQ ID NO: 33 (ZIKV DVG-L), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

In some embodiments the at least one defective interfering genome is a defective interfering EV71 genome. In some embodiments the at least one defective interfering EV71 genome comprises or consists of a nucleotide sequence selected from the group consisting of: SEQ ID NO: 1 (DG1 : EV71 DVG 293-390), SEQ ID NO: 2 (DG2 : EV71 DVG 294-404), SEQ ID NO: 3 (DG3 : EV71 DVG 1746-2895), SEQ ID NO: 4 (DG4 : EV71 DVG 1752-2894), SEQ ID NO: 5 (DG5 : EV71 DVG 1752-2896), SEQ ID NO: 6 (DG6 : EV71 DVG 1880-6487), SEQ ID NO: 7 (DG7 : EV71 DVG 1880-6488), SEQ ID NO: 8 (DG8 : EV71 DVG 3513-6516), SEQ ID NO: 9 (DG9 : EV71 DVG 5475-5634), SEQ ID NO: 10 (DG10: EV71 DVG 5610-6876), SEQ ID NO: 11 (DG11 : EV71 DVG 5610-6877), SEQ ID NO: 12 (DG12: EV71 DVG 5746-5821), SEQ ID NO: 13 (DG13 : EV71 DVG 6322-6356), SEQ ID NO: 14 (DG14: EV71 DVG 6322-6358), SEQ ID NO: 15 (DG15: EV71 DVG 6728-6779), SEQ ID NO: 16 (DG16: EV71 DVG 6966-7012), SEQ ID NO: 17 (DG17: EV71 DVG 6966-7014), SEQ ID NO: 18 (DG18: EV71 DVG 7098-7122), SEQ ID NO: 19 (DG19: EV71 DVG 7165-7200), SEQ ID NO: 20 (DG20: EV71 DVG 7165-7201) and SEQ ID NO: 21 (DG21: EV71 DVG 7238-7292), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

In some embodiments the at least one defective interfering genome is a defective interfering RV genome. In some embodiments the at least one defective interfering RV genome comprises or consists of a nucleotide sequence selected from the group consisting of: SEQ ID NO: 55 (RV DVG A01a-TIP-01), SEQ ID NO: 56 (RV DVG A01a-TIP-02), SEQ ID NO: 57 (RV DVG A01a-TIP-03), SEQ ID NO: 58 (RV DVG A01a-TIP-04), SEQ ID NO: 59 (RV DVG A01a-TIP-05), SEQ ID NO: 60 (RV DVG A01a-TIP-06), SEQ ID NO: 62 (RV DVG A16-TIP-01), SEQ ID NO: 63 (RV DVG A16-TIP-02), SEQ ID NO: 64 (RV DVG A16-TIP-03), SEQ ID NO: 65 (RV DVG A16-TIP-04), SEQ ID NO: 66 (RV DVG A16-TIP-05), SEQ ID NO: 89 (RV DVG C15-TIP-01), SEQ ID NO: 90 (RV DVG C15-TIP-02), SEQ ID NO: 91 (RV DVG C15-TIP-03), SEQ ID NO: 92 (RV DVG C15-TIP-04), SEQ ID NO: 68 (RV DVG B14-TIP-01), SEQ ID NO: 69 (RV DVG B14-TIP-02), SEQ ID NO: 70 (RV DVG B14-TIP-03), SEQ ID NO: 71 (RV DVG B14-TIP-04), SEQ ID NO: 72 (RV DVG B14-TIP-05), SEQ ID NO: 73 (RV DVG B14-TIP-06), SEQ ID NO: 74 (RV DVG B14-TIP-07), SEQ ID NO: 75 (RV DVG B14-TIP-08), SEQ ID NO: 76 (RV DVG B14-TIP-09), SEQ ID NO: 77 (RV DVG B14-TIP-10), SEQ ID NO: 78 (RV DVG B14-TIP-11), SEQ ID NO: 79 (RV DVG B14-TIP-12), SEQ ID NO: 80 (RV DVG B14-TIP-13), SEQ ID NO: 81 (RV DVG B14-TIP-14), SEQ ID NO: 82 (RV DVG B14-TIP-15), SEQ ID NO: 83 (RV DVG B14-TIP-16), SEQ ID NO: 84 (RV DVG B14-TIP-17), SEQ ID NO: 85 (RV DVG B14-TIP-18), SEQ ID NO: 86 (RV DVG B14-TIP-19) and SEQ ID NO: 87 (RV DVG B14-TIP-20), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

In some embodiments the at least one defective interfering genome is a defective interfering YFV genome. In some embodiments the at least one defective interfering YFV genome comprises or consists of a nucleotide sequence selected from the group consisting of: SEQ ID NO:93 (YFV DVG-A), SEQ ID NO: 94 (YFV DVG-B), SEQ ID NO: 95 (YFV DVG-C), SEQ ID NO: 96 (YFV DVG-D), SEQ ID NO: 97 (YFV DVG-E), SEQ ID NO: 98 (YFV DVG-F), SEQ ID NO: 99 (YFV DVG-G), SEQ ID NO: 100 (YFV DVG-H), SEQ ID NO: 101 (YFV DVG-I), SEQ ID NO: 102 (YFV DVG-J), SEQ ID NO: 103 (YFV DVG-K) and SEQ ID NO: 104 (YFV DVG-L), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

In some embodiments the at least one defective interfering genome is a defective interfering WNV genome. In some embodiments the at least one defective interfering WNV genome comprises or consists of a nucleotide sequence selected from the group consisting of: SEQ ID NO:105 (WNV DVG-1), SEQ ID NO: 106 (WNV DVG-2), SEQ ID NO: 107 (WNV DVG-3), SEQ ID NO: 108 (WNV DVG-4), SEQ ID NO: 109 (WNV DVG-5) and SEQ ID NO: 110 (WNV DVG-6), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

In some embodiments the at least one defective interfering genome is a defective interfering CV genome, for example a defective interfering SARS-CoV-2 genome. In some embodiments the at least one defective interfering SARS-CoV-2 genome comprises or consists of a nucleotide sequence selected from the group consisting of: SEQ ID NO: 111 (SARS-CoV-2 DVG_1), SEQ ID NO: 112 (SARS-CoV-2 DVG_2) and SEQ ID NO: 113 (SARS-CoV-2 DVG_3), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

Also provided is a defective interfering genome DVG or defective interfering particle of the invention for use in a method of treatment of CHIKV infection to a subject, wherein the defective interfering genome comprises or consists of a nucleotide sequence selected from the group consisting of: SEQ ID NO: 34 (CHIKV DVG-IV1), SEQ ID NO: 35 (CHIKV DVG-IV2), SEQ ID NO: 36 (CHIKV DVG-IV3), SEQ ID NO: 37 (CHIKV DVG-IV4), SEQ ID NO: 38 (CHIKV DVG-IH1), SEQ ID NO: 39 (CHIKV DVG-IU1), SEQ ID NO: 40 (CHIKV DVG-CV1), SEQ ID NO: 41 (CHIKV DVG-CV2), SEQ ID NO: 42 (CHIKV DVG-CV3), SEQ ID NO: 43 (CHIKV DVG-CV4), SEQ ID NO: 44 (CHIKV DVG-CH1), SEQ ID NO: 45 (CHIKV DVG-CH2), SEQ ID NO: 46 (CHIKV DVG-CH3), SEQ ID NO: 47 (CHIKV DVG-CM1), SEQ ID NO: 48 (CHIKV DVG-CU1), SEQ ID NO: 49 (CHIKV DVG-CU2), SEQ ID NO: 50 (CHIKV DVG-CA1), SEQ ID NO: 51 (CHIKV DVG-CA2), SEQ ID NO: 52 (CHIKV DVG-CA3) and SEQ ID NO: 53 (CHIKV DVG-CA4), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

Also provided is a defective interfering genome DVG or defective interfering particle of the invention for use in a method of treatment of ZIKV infection to a subject, wherein the defective interfering genome comprises or consists of a nucleotide sequence selected from the group consisting of: SEQ ID NO: 22 (ZIKV DVG-A), SEQ ID NO: 23 (ZIKV DVG-B), SEQ ID NO: 24 (ZIKV DVG-C), SEQ ID NO: 25 (ZIKV DVG-D), SEQ ID NO: 26 (ZIKV DVG-E), SEQ ID NO: 27 (ZIKV DVG-F), SEQ ID NO: 28 (ZIKV DVG-G), SEQ ID NO: 29 (ZIKV DVG-H), SEQ ID NO: 30 (ZIKV DVG-I), SEQ ID NO: 31 (ZIKV DVG-J), SEQ ID NO: 32 (ZIKV DVG-K) and SEQ ID NO: 33 (ZIKV DVG-L), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

Also provided is a defective interfering genome DVG or defective interfering particle of the invention for use in a method of treatment of EV71 infection to a subject, wherein the defective interfering genome comprises or consists of a nucleotide sequence selected from the group consisting of: SEQ ID NO: 1 (DG1 : EV71 DVG 293-390), SEQ ID NO: 2 (DG2 : EV71 DVG 294-404), SEQ ID NO: 3 (DG3 : EV71 DVG 1746-2895), SEQ ID NO: 4 (DG4 : EV71 DVG 1752-2894), SEQ ID NO: 5 (DG5 : EV71 DVG 1752-2896), SEQ ID NO: 6 (DG6 : EV71 DVG 1880-6487), SEQ ID NO: 7 (DG7 : EV71 DVG 1880-6488), SEQ ID NO: 8 (DG8 : EV71 DVG 3513-6516), SEQ ID NO: 9 (DG9 : EV71 DVG 5475-5634), SEQ ID NO: 10 (DG10: EV71 DVG 5610-6876), SEQ ID NO: 11 (DG11 : EV71 DVG 5610-6877), SEQ ID NO: 12 (DG12: EV71 DVG 5746-5821), SEQ ID NO: 13 (DG13 : EV71 DVG 6322-6356), SEQ ID NO: 14 (DG14: EV71 DVG 6322-6358), SEQ ID NO: 15 (DG15: EV71 DVG 6728-6779), SEQ ID NO: 16 (DG16: EV71 DVG 6966-7012), SEQ ID NO: 17 (DG17: EV71 DVG 6966-7014), SEQ ID NO: 18 (DG18: EV71 DVG 7098-7122), SEQ ID NO: 19 (DG19: EV71 DVG 7165-7200), SEQ ID NO: 20 (DG20: EV71 DVG 7165-7201) and SEQ ID NO: 21 (DG21: EV71 DVG 7238-7292), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

Also provided is a defective interfering genome DVG or defective interfering particle of the invention for use in a method of treatment of RV infection to a subject, wherein the defective interfering genome comprises or consists of a nucleotide sequence selected from the group consisting of: SEQ ID NO: 55 (RV DVG A01a-TIP-01), SEQ ID NO: 56 (RV DVG A01a-TIP-02), SEQ ID NO: 57 (RV DVG A01a-TIP-03), SEQ ID NO: 58 (RV DVG A01a-TIP-04), SEQ ID NO: 59 (RV DVG A01a-TIP-05), SEQ ID NO: 60 (RV DVG A01a-TIP-06), SEQ ID NO: 62 (RV DVG A16-TIP-01), SEQ ID NO: 63 (RV DVG A16-TIP-02), SEQ ID NO: 64 (RV DVG A16-TIP-03), SEQ ID NO: 65 (RV DVG A16-TIP-04), SEQ ID NO: 66 (RV DVG A16-TIP-05), SEQ ID NO: 89 (RV DVG C15-TIP-01), SEQ ID NO: 90 (RV DVG C15-TIP-02), SEQ ID NO: 91 (RV DVG C15-TIP-03), SEQ ID NO: 92 (RV DVG C15-TIP-04), SEQ ID NO: 68 (RV DVG B14-TIP-01), SEQ ID NO: 69 (RV DVG B14-TIP-02), SEQ ID NO: 70 (RV DVG B14-TIP-03), SEQ ID NO: 71 (RV DVG B14-TIP-04), SEQ ID NO: 72 (RV DVG B14-TIP-05), SEQ ID NO: 73 (RV DVG B14-TIP-06), SEQ ID NO: 74 (RV DVG B14-TIP-07), SEQ ID NO: 75 (RV DVG B14-TIP-08), SEQ ID NO: 76 (RV DVG B14-TIP-09), SEQ ID NO: 77 (RV DVG B14-TIP-10), SEQ ID NO: 78 (RV DVG B14-TIP-11), SEQ ID NO: 79 (RV DVG B14-TIP-12), SEQ ID NO: 80 (RV DVG B14-TIP-13), SEQ ID NO: 81 (RV DVG B14-TIP-14), SEQ ID NO: 82 (RV DVG B14-TIP-15), SEQ ID NO: 83 (RV DVG B14-TIP-16), SEQ ID NO: 84 (RV DVG B14-TIP-17), SEQ ID NO: 85 (RV DVG B14-TIP-18), SEQ ID NO: 86 (RV DVG B14-TIP-19) and SEQ ID NO: 87 (RV DVG B14-TIP-20), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

Also provided is a defective interfering genome DVG or defective interfering particle of the invention for use in a method of treatment of YFV infection to a subject, wherein the defective interfering genome comprises or consists of a nucleotide sequence selected from the group consisting of: SEQ ID NO:93 (YFV DVG-A), SEQ ID NO: 94 (YFV DVG-B), SEQ ID NO: 95 (YFV DVG-C), SEQ ID NO: 96 (YFV DVG-D), SEQ ID NO: 97 (YFV DVG-E), SEQ ID NO: 98 (YFV DVG-F), SEQ ID NO: 99 (YFV DVG-G), SEQ ID NO: 100 (YFV DVG-H), SEQ ID NO: 101 (YFV DVG-I), SEQ ID NO: 102 (YFV DVG-J), SEQ ID NO: 103 (YFV DVG-K) and SEQ ID NO: 104 (YFV DVG-L), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

Also provided is a defective interfering genome DVG or defective interfering particle of the invention for use in a method of treatment of WNV infection to a subject, wherein the defective interfering genome comprises or consists of a nucleotide sequence selected from the group consisting of: SEQ ID NO:105 (WNV DVG-1), SEQ ID NO: 106 (WNV DVG-2), SEQ ID NO: 107 (WNV DVG-3), SEQ ID NO: 108 (WNV DVG-4), SEQ ID NO: 109 (WNV DVG-5) and SEQ ID NO: 110 (WNV DVG-6), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

Also provided is a defective interfering genome DVG or defective interfering particle of the invention for use in a method of treatment of a CV infection to a subject, wherein the CV infection is caused by SARS-CoV-2, and wherein the defective interfering genome comprises or consists of a nucleotide sequence selected from the group consisting of: SEQ ID NO: 111 (SARS-CoV-2 DVG_1), SEQ ID NO: 112 (SARS-CoV-2 DVG_2) and SEQ ID NO: 113 (SARS-CoV-2 DVG_3), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

Also provided is a pharmaceutical, immunogenic, or therapeutic composition, comprising at least one DVG according to the invention, and a pharmaceutically acceptable carrier.

Also provided is a pharmaceutical, immunogenic, or therapeutic composition, comprising at least one defective interfering particle according to the invention, and a pharmaceutically acceptable carrier.

Also provided is a vaccine comprising an immunogenic composition of the invention.

Also provided is the use of a DVG of the invention, or a defective interfering particle of the invention, for the preparation of a drug for the treatment of a patient infected with a target virus of the DVG or defective interfering particle.

Also provided is the use of a DVG of the invention, or a defective interfering particle of the invention, for the preparation of a drug for the treatment of a patient infected with CHIKV.

Also provided is the use of a DVG of the invention, or a defective interfering particle of the invention, for the preparation of a drug for the treatment of a patient infected with ZIKV.

Also provided is the use of a DVG of the invention, or a defective interfering particle of the invention, for the preparation of a drug for the treatment of a patient infected with EV71.

Also provided is the use of a DVG of the invention, or a defective interfering particle of the invention, for the preparation of a drug for the treatment of a patient infected with RV.

Also provided is the use of a DVG of the invention, or a defective interfering particle of the invention, for the preparation of a drug for the treatment of a patient infected with YFV.

Also provided is the use of a DVG of the invention, or a defective interfering particle of the invention, for the preparation of a drug for the treatment of a patient infected with WNV.

Also provided is the use of a DVG of the invention, or a defective interfering particle of the invention, for the preparation of a drug for the treatment of a patient infected with CV, in particular by SARS-CoV-2.

Also provided is a polynucleotide comprising or consisting of a nucleotide sequence selected from the group consisting of: SEQ ID NO: 1 (DG1 : EV71 DVG 293-390), SEQ ID NO: 2 (DG2 : EV71 DVG 294-404), SEQ ID NO: 3 (DG3 : EV71 DVG 1746-2895), SEQ ID NO: 4 (DG4 : EV71 DVG 1752-2894), SEQ ID NO: 5 (DG5 : EV71 DVG 1752-2896), SEQ ID NO: 6 (DG6 : EV71 DVG 1880-6487), SEQ ID NO: 7 (DG7 : EV71 DVG 1880-6488), SEQ ID NO: 8 (DG8 : EV71 DVG 3513-6516), SEQ ID NO: 9 (DG9 : EV71 DVG 5475-5634), SEQ ID NO: 10 (DG10: EV71 DVG 5610-6876), SEQ ID NO: 11 (DG11 : EV71 DVG 5610-6877), SEQ ID NO: 12 (DG12: EV71 DVG 5746-5821), SEQ ID NO: 13 (DG13 : EV71 DVG 6322-6356), SEQ ID NO: 14 (DG14: EV71 DVG 6322-6358), SEQ ID NO: 15 (DG15: EV71 DVG 6728-6779), SEQ ID NO: 16 (DG16: EV71 DVG 6966-7012), SEQ ID NO: 17 (DG17: EV71 DVG 6966-7014), SEQ ID NO: 18 (DG18: EV71 DVG 7098-7122), SEQ ID NO: 19 (DG19: EV71 DVG 7165-7200), SEQ ID NO: 20 (DG20: EV71 DVG 7165-7201), SEQ ID NO: 21 (DG21: EV71 DVG 7238-7292), SEQ ID NO: 22 (ZIKV DVG-A), SEQ ID NO: 23 (ZIKV DVG-B), SEQ ID NO: 24 (ZIKV DVG-C), SEQ ID NO: 25 (ZIKV DVG-D), SEQ ID NO: 26 (ZIKV DVG-E), SEQ ID NO: 27 (ZIKV DVG-F), SEQ ID NO: 28 (ZIKV DVG-G), SEQ ID NO: 29 (ZIKV DVG-H), SEQ ID NO: 30 (ZIKV DVG-I), SEQ ID NO: 31 (ZIKV DVG-J), SEQ ID NO: 32 (ZIKV DVG-K), SEQ ID NO: 33 (ZIKV DVG-L), SEQ ID NO: 34 (CHIKV DVG-IV1), SEQ ID NO: 35 (CHIKV DVG-IV2), SEQ ID NO: 36 (CHIKV DVG-IV3), SEQ ID NO: 37 (CHIKV DVG-IV4), SEQ ID NO: 38 (CHIKV DVG-IH1), SEQ ID NO: 39 (CHIKV DVG-IU1), SEQ ID NO: 40 (CHIKV DVG-CV1), SEQ ID NO: 41 (CHIKV DVG-CV2), SEQ ID NO: 42 (CHIKV DVG-CV3), SEQ ID NO: 43 (CHIKV DVG-CV4), SEQ ID NO: 44 (CHIKV DVG-CH1), SEQ ID NO: 45 (CHIKV DVG-CH2), SEQ ID NO: 46 (CHIKV DVG-CH3), SEQ ID NO: 47 (CHIKV DVG-CM1), SEQ ID NO: 48 (CHIKV DVG-CU1), SEQ ID NO: 49 (CHIKV DVG-CU2), SEQ ID NO: 50 (CHIKV DVG-CA1), SEQ ID NO: 51 (CHIKV DVG-CA2), SEQ ID NO: 52 (CHIKV DVG-CA3), SEQ ID NO: 53 (CHIKV DVG-CA4), SEQ ID NO: 55 (RV DVG A01a-TIP-01), SEQ ID NO: 56 (RV DVG A01a-TIP-02), SEQ ID NO: 57 (RV DVG A01a-TIP-03), SEQ ID NO: 58 (RV DVG A01a-TIP-04), SEQ ID NO: 59 (RV DVG A01a-TIP-05), SEQ ID NO: 60 (RV DVG A01a-TIP-06), SEQ ID NO: 62 (RV DVG A16-TIP-01), SEQ ID NO: 63 (RV DVG A16-TIP-02), SEQ ID NO: 64 (RV DVG A16-TIP-03), SEQ ID NO: 65 (RV DVG A16-TIP-04), SEQ ID NO: 66 (RV DVG A16-TIP-05), SEQ ID NO: 89 (RV DVG C15-TIP-01), SEQ ID NO: 90 (RV DVG C15-TIP-02), SEQ ID NO: 91 (RV DVG C15-TIP-03), SEQ ID NO: 92 (RV DVG C15-TIP-04), SEQ ID NO: 68 (RV DVG B14-TIP-01), SEQ ID NO: 69 (RV DVG B14-TIP-02), SEQ ID NO: 70 (RV DVG B14-TIP-03), SEQ ID NO: 71 (RV DVG B14-TIP-04), SEQ ID NO: 72 (RV DVG B14-TIP-05), SEQ ID NO: 73 (RV DVG B14-TIP-06), SEQ ID NO: 74 (RV DVG B14-TIP-07), SEQ ID NO: 75 (RV DVG B14-TIP-08), SEQ ID NO: 76 (RV DVG B14-TIP-09), SEQ ID NO: 77 (RV DVG B14-TIP-10), SEQ ID NO: 78 (RV DVG B14-TIP-11), SEQ ID NO: 79 (RV DVG B14-TIP-12), SEQ ID NO: 80 (RV DVG B14-TIP-13), SEQ ID NO: 81 (RV DVG B14-TIP-14), SEQ ID NO: 82 (RV DVG B14-TIP-15), SEQ ID NO: 83 (RV DVG B14-TIP-16), SEQ ID NO: 84 (RV DVG B14-TIP-17), SEQ ID NO: 85 (RV DVG B14-TIP-18), SEQ ID NO: 86 (RV DVG B14-TIP-19), and SEQ ID NO: 87 (RV DVG B14-TIP-20), SEQ ID NO:93 (YFV DVG-A), SEQ ID NO: 94 (YFV DVG-B), SEQ ID NO: 95 (YFV DVG-C), SEQ ID NO: 96 (YFV DVG-D), SEQ ID NO: 97 (YFV DVG-E), SEQ ID NO: 98 (YFV DVG-F), SEQ ID NO: 99 (YFV DVG-G), SEQ ID NO: 100 (YFV DVG-H), SEQ ID NO: 101 (YFV DVG-I), SEQ ID NO: 102 (YFV DVG-J), SEQ ID NO: 103 (YFV DVG-K), SEQ ID NO: 104 (YFV DVG-L), SEQ ID NO:105 (WNV DVG-1), SEQ ID NO: 106 (WNV DVG-2), SEQ ID NO: 107 (WNV DVG-3), SEQ ID NO: 108 (WNV DVG-4), SEQ ID NO: 109 (WNV DVG-5), SEQ ID NO: 110 (WNV DVG-6), SEQ ID NO: 111 (SARS-CoV-2 DVG_1), SEQ ID NO: 112 (SARS-CoV-2 DVG_2) and SEQ ID NO: 113 (SARS-CoV-2 DVG_3), or having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

Also provided is an expression vector or a plasmid comprising a polynucleotide of the invention.

Also provided is a cell line producing a DVG according to the invention.

The invention will be more fully understood by reference to the following examples, which are non-limiting.

### EXAMPLES

### Example 1: Methods

### Growth of Viruses

Traditionally, DVGs were observed when virus is passaged at high multiplicity of infection (MOI). Most published work is based on identifying DVGs from few passages and few replicates, resulting in the identification of a single most abundant DVG, generally by methods that biased towards identifying the shortest DVGs with the largest deletions (e.g. by RT-PCR amplification using primers spanning the 5′ and 3′ ends of RNA genomes). This led to the notion that only a few DVG are generated for any given virus. We find that DVGs number is in the hundreds to thousands, in terms of sequence; and that the best candidate DVGs for interference are not necessarily the single most abundant DVG that appears in a single growth condition. In our method, wild-type virus is passaged under both low (control) and high MOI conditions, as well as in mutagenic conditions that we have found to increase the generation of defective genomes. These mutagenic conditions include either mutator variants of viruses that generate more errors than the native wild-type virus, or extrinsic treatments that increase these errors rates (for example, base analogs such as ribavirin, 5-fluorouracil, 5-azacytidine, T705 or other treatments such as increasing temperature or supplementing media with Mn++). Viruses are passaged in high biological replicate numbers (3, 12, 24 or 36) for up to 10 passages. We observed that 5 passages suffice to generate the required data. We also recommend using a variety of cell types or different hosts, since we observe differences in the types of DVGs that are favored between cell lines and hosts.

### Identifying DVG Candidates

The viruses from the passage series are deep sequenced to characterize every deletion or rearrangement that occurs across the entire genome. The number of reads describing a specific deletion/rearrangement relative to the total number of reads at that position is used as a proxy of the frequency at which that deletion is present in the entire virus population. Hundreds to thousands of different DVGs are found for any given virus using this method. We use computational tools to identify which of these DVGs occur with high frequency and abundance, and which are redundant between replicates, to select the 10-20 best candidates for further testing.

We use the following criteria and rationale to predict which DVGs would make the best TIP candidates, based on the assumption that a good candidate will have relatively high replication fitness compared to wild-type, will be encapsidated or passaged from cell to cell alongside wild-type, will compete and interfere with wild-type, will retain domains and functions required for propagation, will retain fitness in different cell types and hosts:

DVGs that occur at higher frequency in a population, are most likely to be viable (able to replicate or be replicated by wild-type virus) and have a higher likelihood of competing with wild-type virus.

DVGs that occur across a passage series, are most likely to be able to compete with wild-type, and be encapsidated into VLPs, to infect new cells.

DVGs that occur in more than three biological replicates, are more likely to have a high enough fitness to compete with wild-type, and likely have retained the structural and functional elements required for replication and propagation.

DVGs that occur in more than one cell type or host, are more likely to be viable in different cell conditions.

DVGs that occur at higher frequency in high MOI or mutagenic conditions versus low MOI conditions, are more likely to be true competitors with wild-type virus.

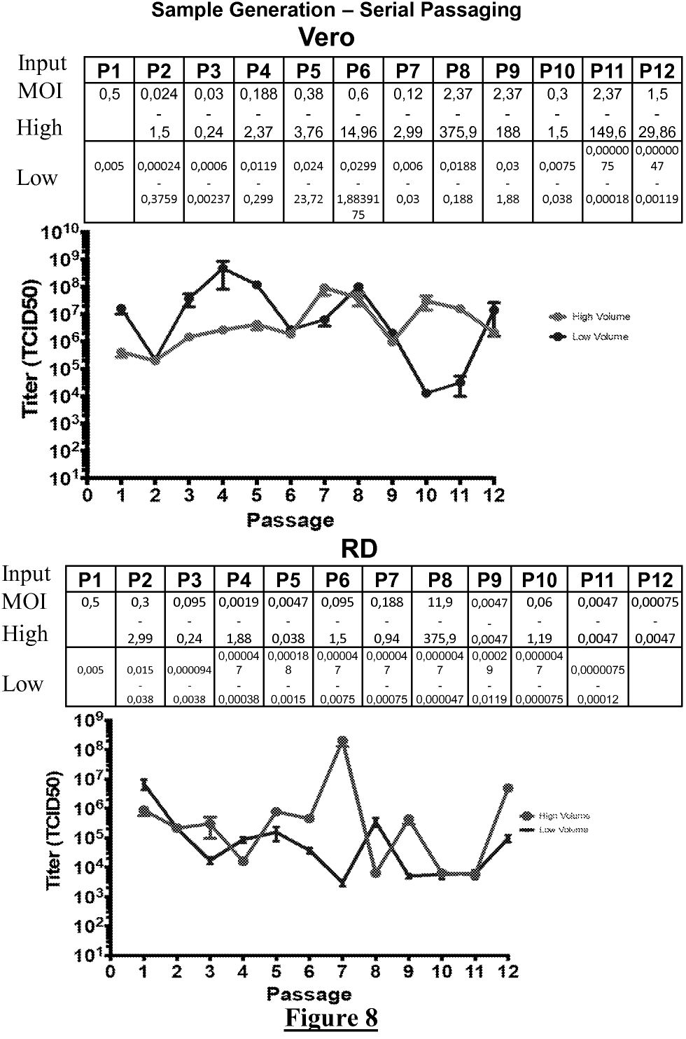

Additionally, we match the nucleotide position of start and stop of the deletion (the breakpoints) with the predicted RNA structure of the genome (See Example 2, FIGS. 8-22). We found that while small deletions occur at random across the entire genome, larger deletions in the DVG candidates that satisfy the criteria above, occur at key structural sites. That is, the predicted RNA structure of a virus can be used to predict where a large deletion will occur, and whether it will result in a viable DVG candidate.

We observed that DVGs may interfere and operate by more than one mechanism. The classic view is that shorter DVGs steal replication machinery from wild-type and outcompete wild-type because their shorter genomes replicate faster. However, additional mechanisms that we have observed include: DVGs that alter the stoichiometry of viral proteins available to wild-type by providing more of some viral proteins than is normally required; DVGs that generate misfolded proteins that induce cellular responses and danger signals; DVGs that encode faulty proteins that result in non-viable wild-type viruses by ‘poisoning’ wild-type replication and assembly; DVGs that induce innate immunity and other cellular responses because they are more readily sensed as danger signals. Ideal DVGs could present one, some or all of these mechanisms or be composed of a cocktail of DVGs each manifesting some of these mechanisms.

Using this approach, we have obtained the total DVG profiles (hundreds to thousands) of the following viruses and strains:
Chikungunya virus, Caribbean and Indian Ocean islands strain;
Zika virus, African strain;
Enterovirus A71;
Rhinovirus A01a, A16, B14, and C15;
Yellow fever virus 17D vaccine strain and Asibi; and
West Nile virus.
Coronavirus, in particular SARS-CoV-2 strain (BetaCoV/ France/IDF0372/2020).

From these lists, using the selection criteria mentioned above, we identified the top candidates for each type of virus. The top sequences of the top candidates are provided in attached information sequence listing. Once the list of top candidates is established, infectious clones of DVGs are made and tested in vitro, and then in vivo. Our results show that this method of identification and down-selection has a very high success rate, where 50-90% of candidates show inhibitory activity against wild-type virus in vitro.

### EXAMPLES OF THE METHOD

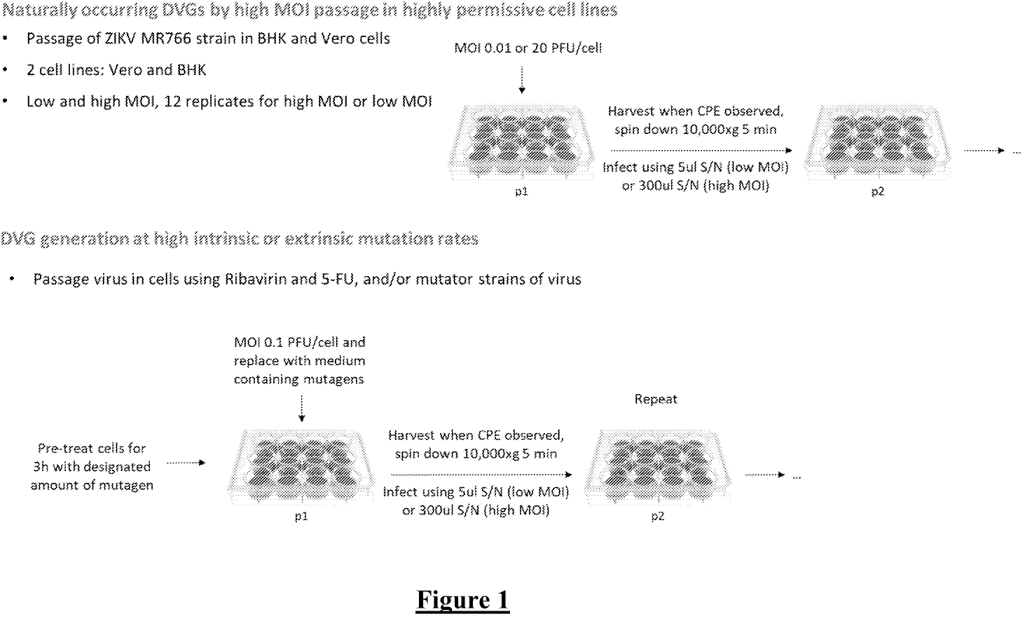

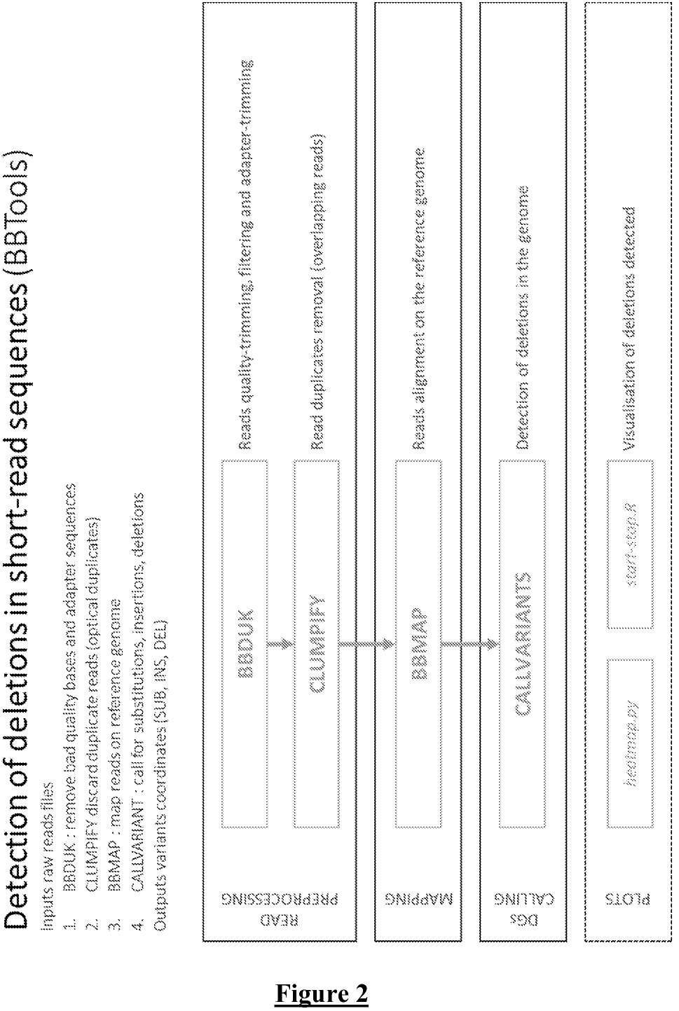

Examples of the method are shown in FIGS. 1 to 7.

FIG. 1 shows passaging methods at low/high MOI or mutagenic conditions.

FIG. 2 shows bioinformatics pipeline to detect deletions.

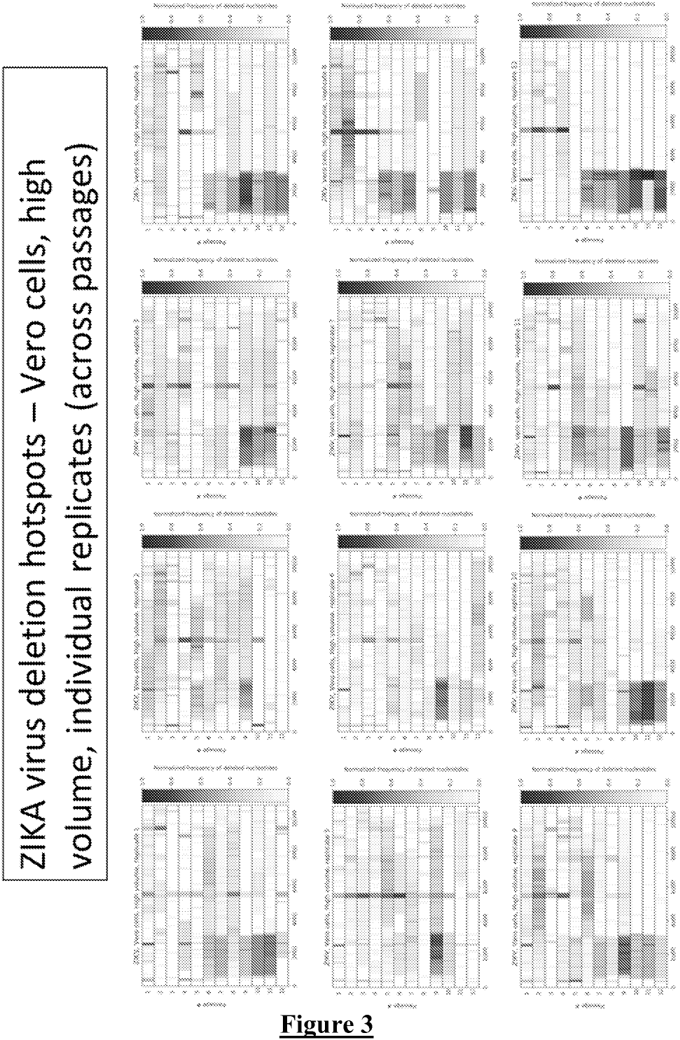

FIG. 3 shows heat map example of deletions.

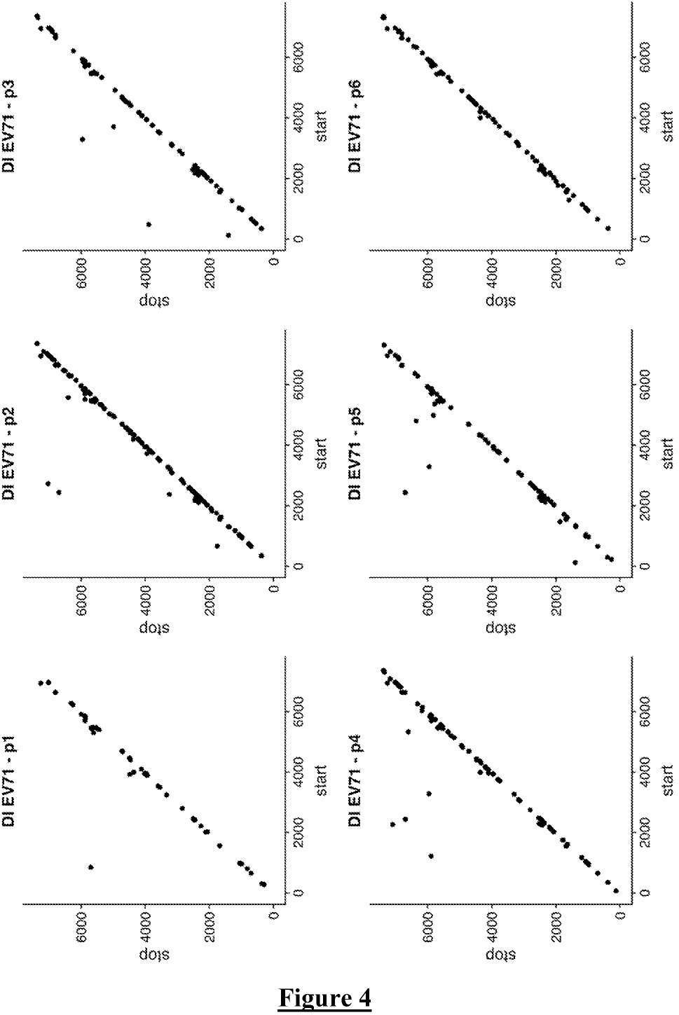

FIG. 4 shows start/stop example.

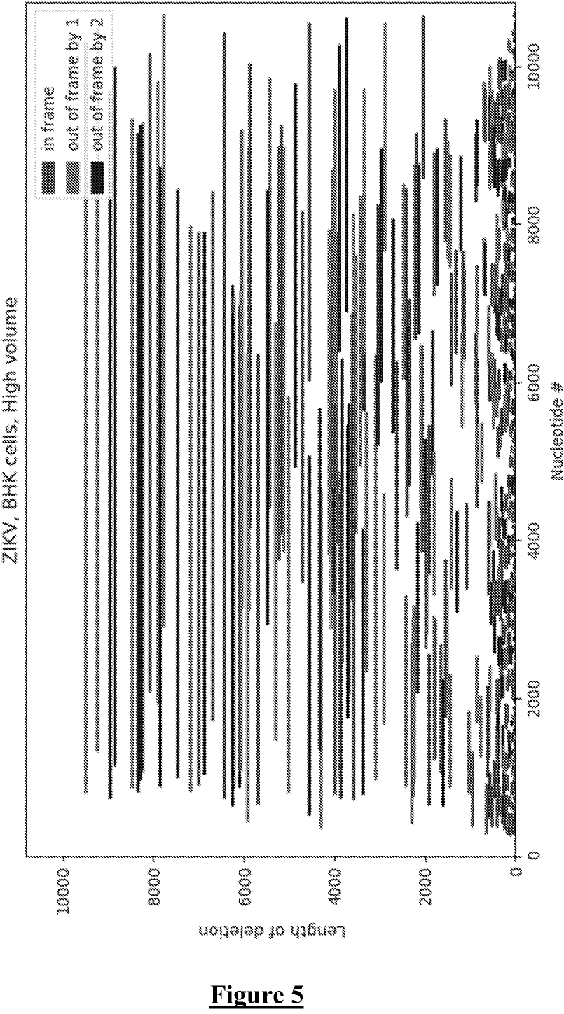

FIG. 5 shows specific deletions in a sample example.

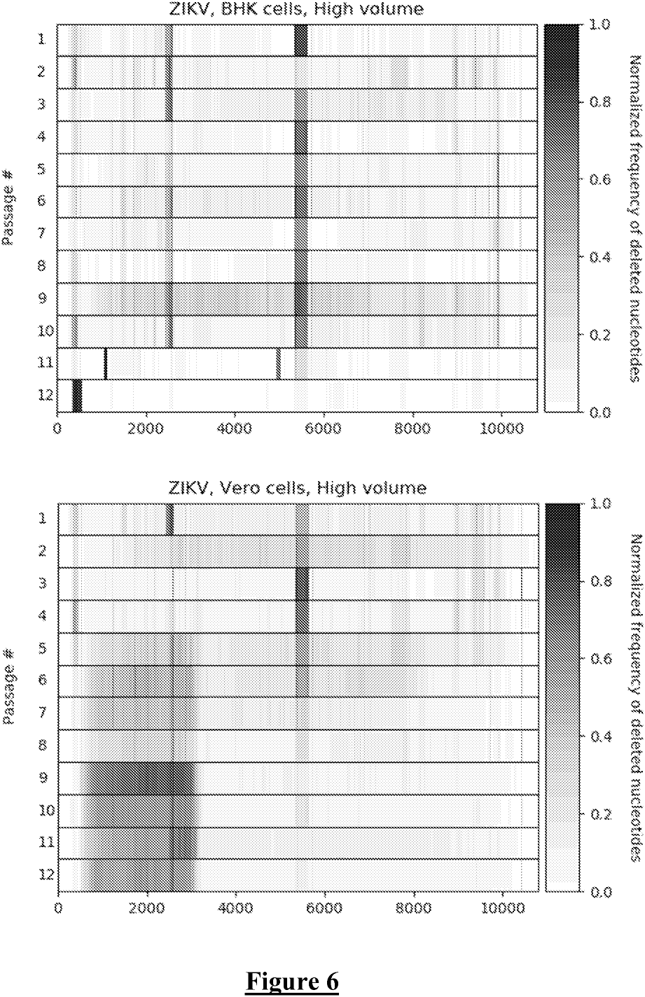

FIG. 6 shows differences in cell types example.

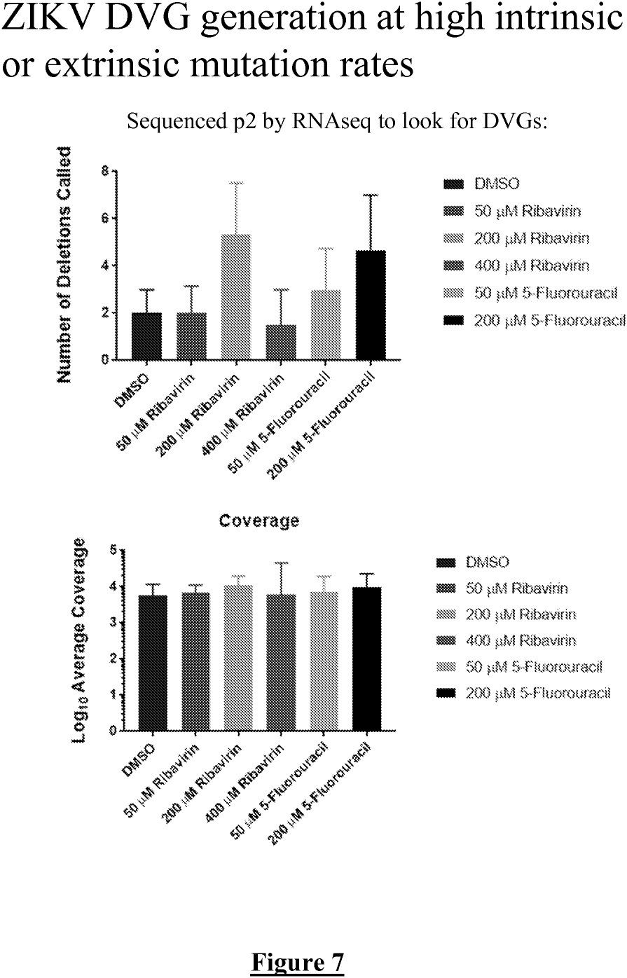

FIG. 7 shows mutagenic conditions sample.

### Example 2: Enterovirus A71 (EV71)

### Generation and Characterization of All DVGs Occurring Within EV71

A 12-well plate was seeded with Vero cells to reach ~90% confluency the next day. The 12 wells were individually transfected with 500 ng pCAGGS-EV71_Sep006 (EV71 Sep006 - GenBank: KX197462.1), using Lipofectamine 2000 (Life Technologies), to generate infectious virus from a stable background. Subsequently Vero cells were infected with 300 µl of the virus supernatant. Or 12-well plates of Vero or RD cells were infected either with a fixed low volume (3 µl) or a high volume (300 µl). Cells were incubated at 37° C., and 5% CO₂ until wells showed >90% CPE. After two freeze-thaw cycles fresh confluent monolayers of cells were infected. This was repeated 12 times using 12 replicates.

The supernatant was processed by centrifugation (13,000 g for 30 min), followed by either RNaseA treatment (1 µl; Thermo; 3 h at 37° C.) or by PEG-precipitation (10 mM HEPES (Sigma), pH 8.0, 0.8% PEG8000 (Sigma), and 50 mM NaCl (Sigma)), followed by another centrifugation of 13,500 x g for 1 h. The RNA was extracted from the cleared supernatant using Trizol (Sigma).

Sample RNA was fragmented using the NEBNext RNA First Strand Buffer (NEB) and heat-fragmentation at 94° C. for 15 min. The RNAseq libraries were generated the NEBNext Ultra II Non-Directional RNA Library Prep Kit for Illumina (NEB) according to the manufacturer’s instructions. To allow multiplexing of the samples, NEBNext Multiplex Oligos for Illumina (NEB) were used to generate indexes. The generated libraries were pooled and sequenced on an Illumina NextSeq500 Sequencer using Illumina NextSeq 500/550 Mid Output Reagent Cartridge v2 and Illumina NextSeq 500/550 Mid Output Flow Cell Cartridge v2.5 using 150 cycle single-end reads. The obtained sequences were analyzed using the BBMap suite-based computational pipeline.

Initially identified DVGs were analyzed by reoccurrence between replicates, passages, and cell lines. This yielded a list of 15 DVGs that were taken forward to be analyzed for interference activity and thus, whether these DVGs could be used as TIPs. Of the 15 identified DVGs, 6 resulted in a deletion that would result in a nonsense mutation after the breakpoint. As a control, the deletion length of these 6 DVGs were adjusted to remain the open reading frame. Thus, the candidate list assessed was 21 DVGs.

FIG. 8: Serial Passaging of EV71. 12 replicates of either Vero or RD cells were infected with EV71 at fixed volumes of either 3 µl (low) or 300 µl (high). Infections were incubated until cells showed >90% CPE. The virus was serially passaged over 12 passages. Virus titers were measured using TCID50 and the MOI range that could be obtained with these fixed volumes were calculated.

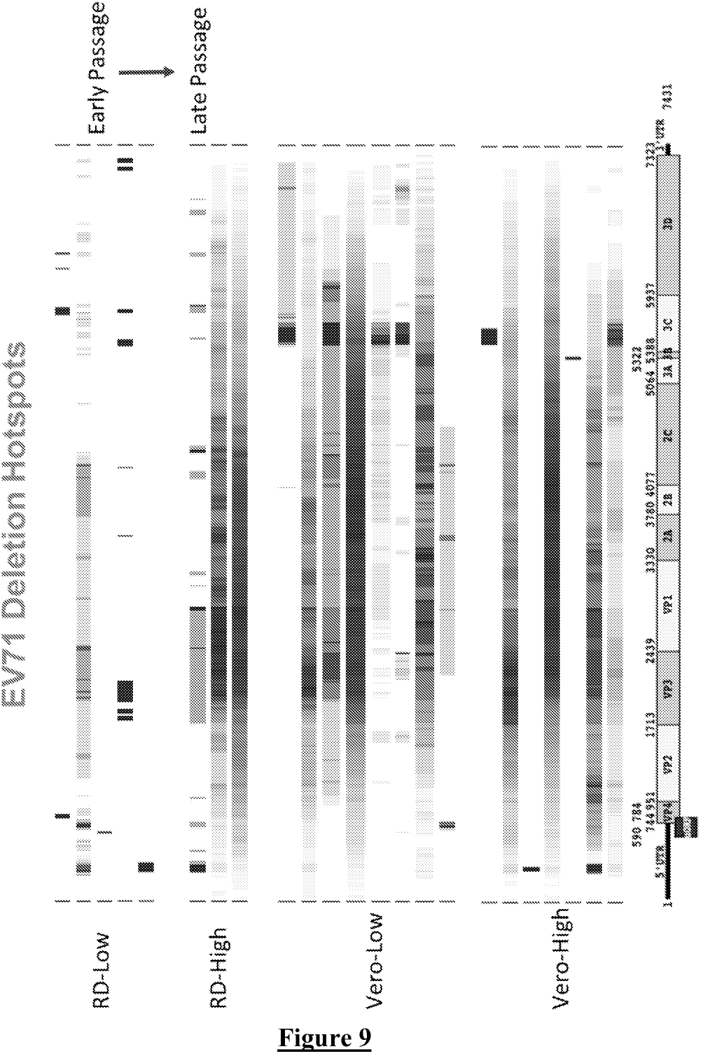

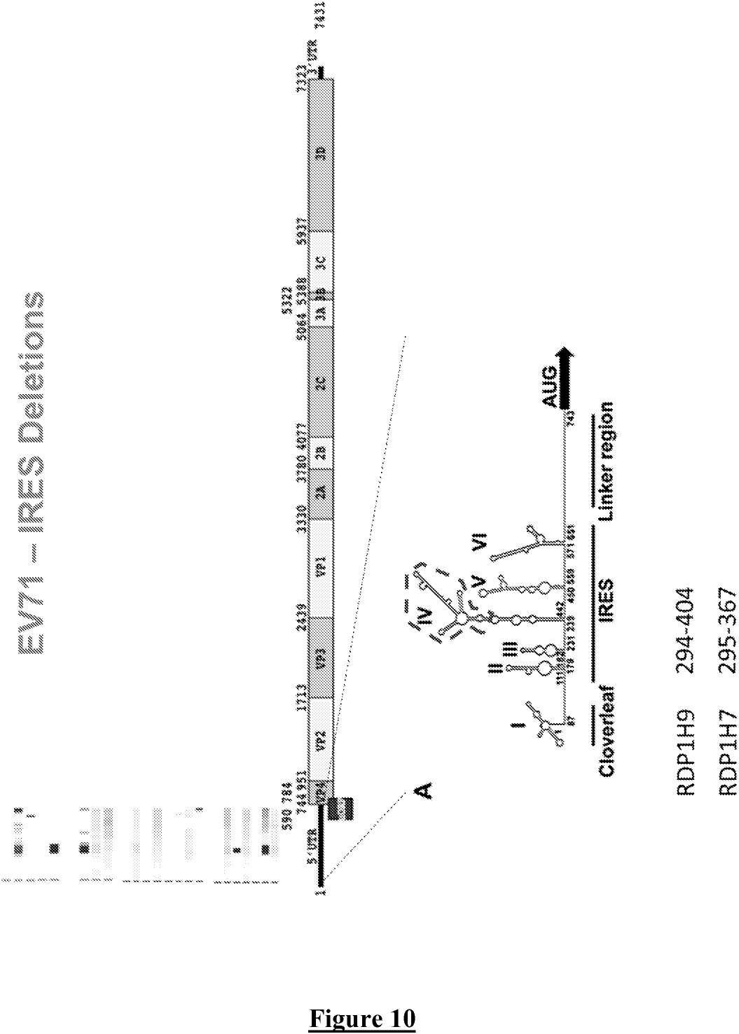

FIG. 9: Deletion Heatmaps of EV71. The samples from the serial passaging were analyzed by RNAseq and deletions were detected using the BBMap-based pipeline. Subsequently the individual replicates in each cell line, condition, and passage were pooled and visualized to show a heatmap of the frequency of each deleted nucleotide relative to the position in the virus genome. Overall the deletion hotspots were relatively small and often had distinct locations in relation to the genome.

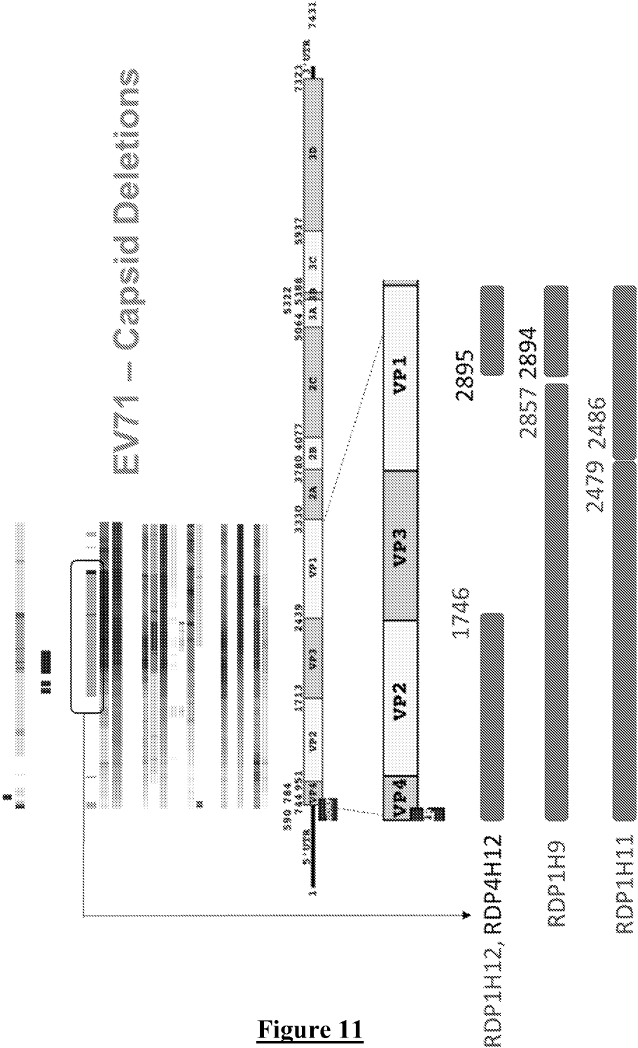

FIG. 10: Deletion Hotspots in the IRES. Aligning the Deletion hotspots to the virus genome, there is a strong hotspot across passages and cell lines that is located in the 5′ UTR of EV71. Closer analysis shows that this hotspot corresponds to the top of stem-loop IV of the IRES (circled in red), which is required to initiate efficient translation of the RNA to the viral polyprotein.

FIG. 11: Deletion Hotspots in the Capsid. Many deletion hotspots of EV71 locate to the capsid region of the genome. This region corresponds to the structural proteins of the virus. Examples of a region that was frequently deleted are marked in the box (black) and the schematic of the deletion in relation to the genome shows deletions in the within the main capsid protein VP1, as well as a larger deletion that spans across the majority of VP3 and VP1.

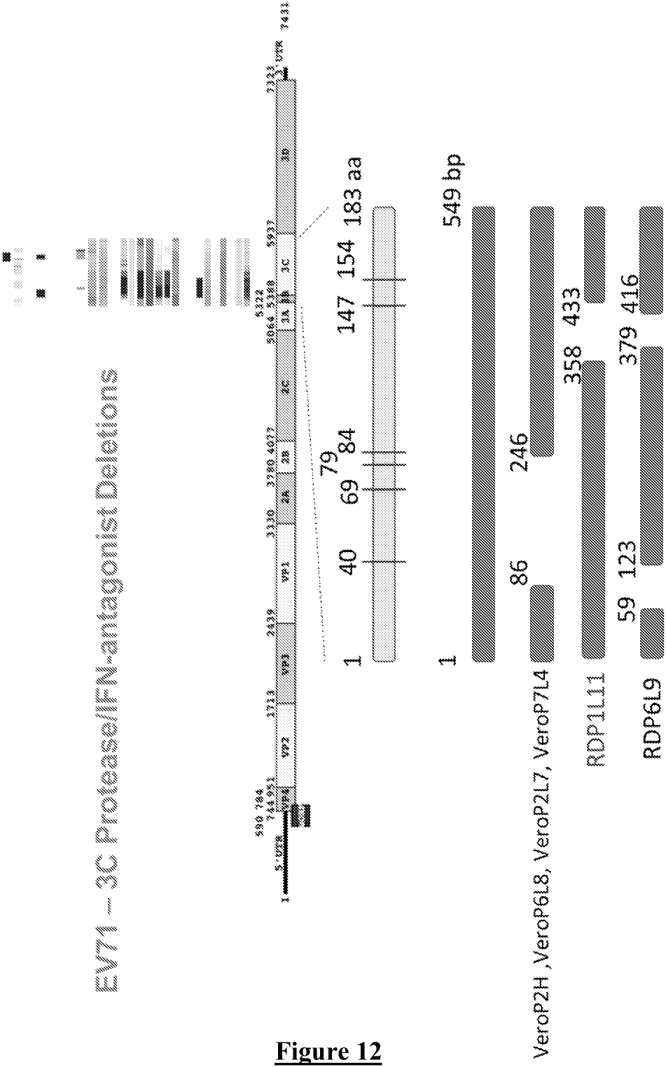

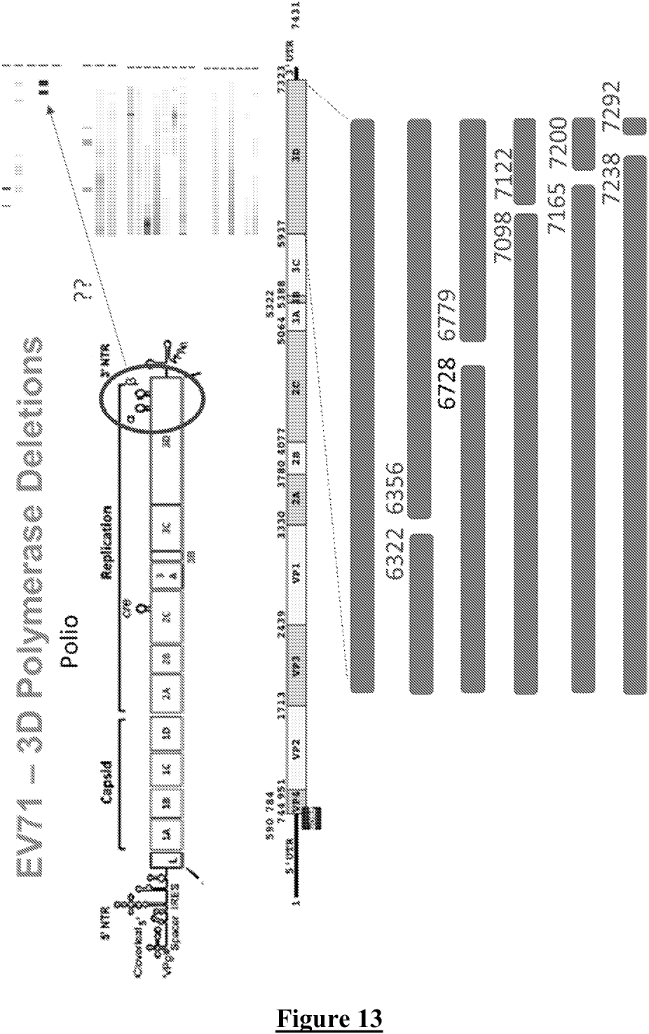

FIG. 12: Deletion Hotspot in 3C. The 3C protein in EV71 has a dual function, it is the virus’ main protease that cleaves the polyprotein into its individual functional proteins. At the same time the 3C protein is also the main interferon antagonist and thus, the main protein that counteracts the cell’s innate immune response. The protein is 183 amino acids in length, while 3C active side of protein consists of at least six residues (marked in red). Interestingly, the deletion hotspots in 3C seem to overlap with the active side, suggesting that deletions would render the protein nonfunctional.

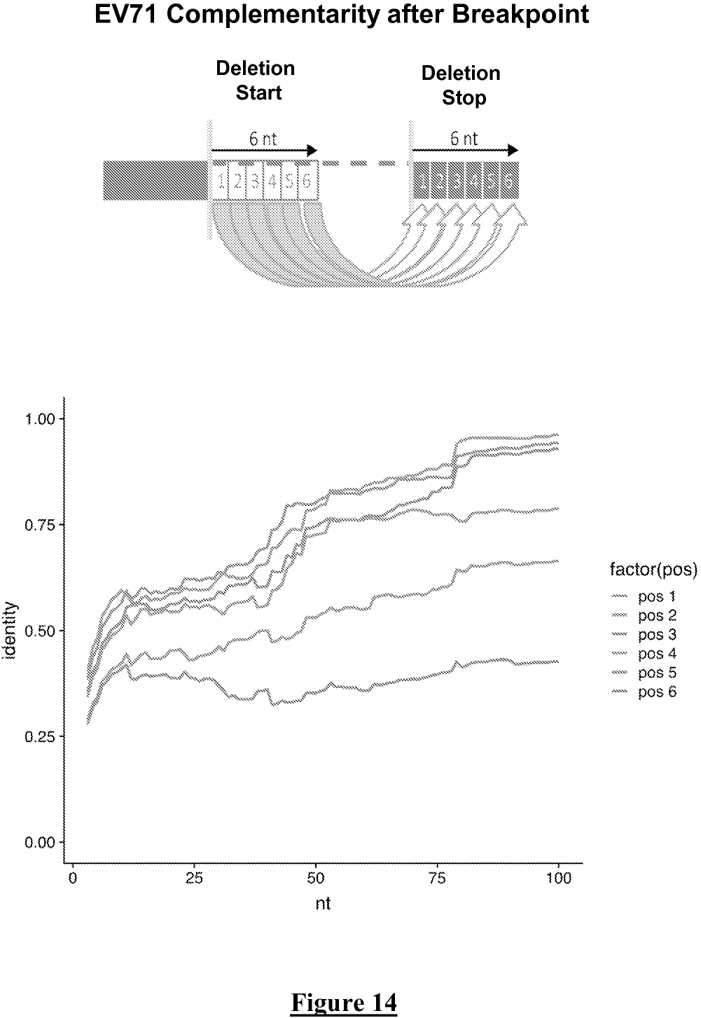

FIG. 13: Deletion Hotspot in 3D. The viral polymerase is located in the 3D protein. The deletion heatmap indicates the presence of a number of different small deletions throughout the protein. Interestingly, there are two heat signals towards the 3′-end of the open-reading frame, which appear to be located within two predicted stem-loop structures, which were also identified in poliovirus and were implicated in the sufficient replication of the virus.

The deletion candidates, as well as other DVGs, were further examined to determine whether the deleted sequences that were observed, correlated with either specific nucleotide signatures or RNA structures.

FIG. 14: Complementarity of Nucleotides after Breakpoints. To analyze the complementarity of the nucleotides around the breakpoints, the nucleotides were assessed individually and positions 1-6 after the start-breakpoint were tested against positions 1-6 after the end-breakpoint. The graph shows the frequency of complementarity on the y-axis and the nucleotide-threshold for the deletions length, i.e. all deletions of this size and bigger, on the x-axis. Deletions of 41 nucleotides and shorter do not rely on complementarity around the breakpoints, which suggests rather a slipping of the polymerase along the genome as mode of action. The frequency of complementarity increases gradually, indicating that the longer the deletion is the more important is complementarity around the breakpoints. For deletions longer than 79 nucleotides, around 95% have the first nucleotide position and around 88.5% the first three nucleotide positions complementary to each other. A random event would be expected to be around 25%.

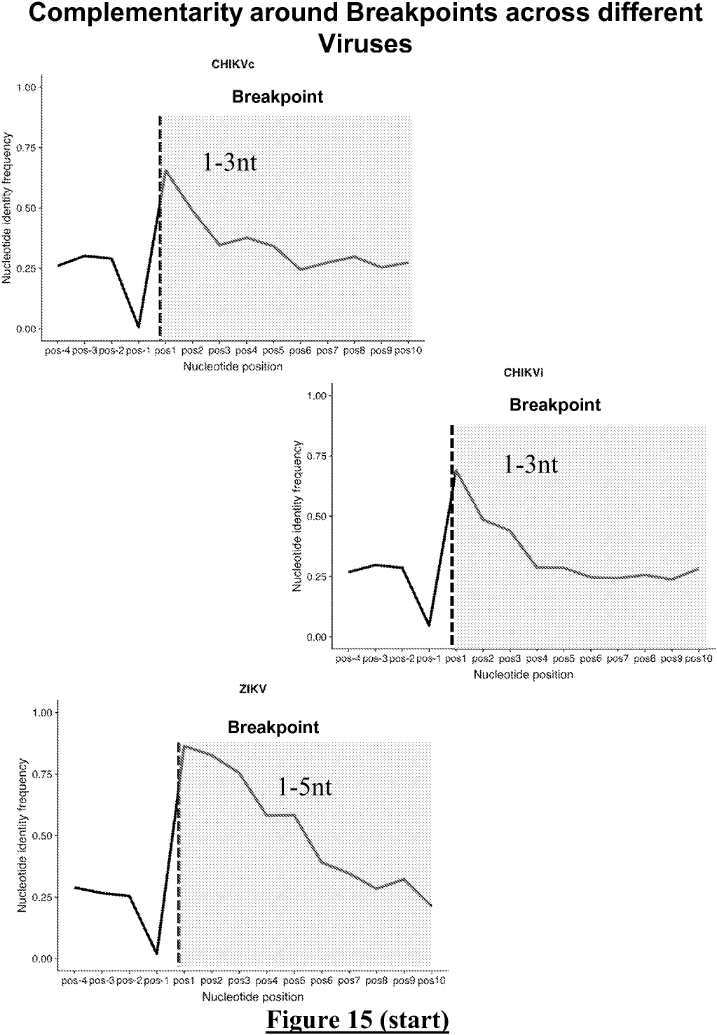

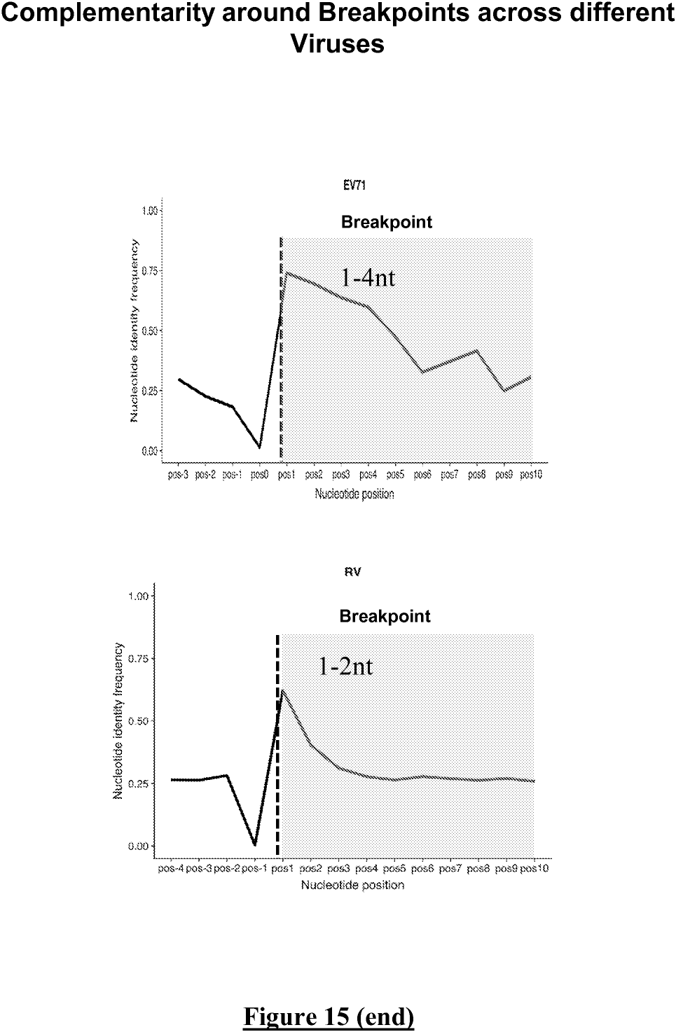

FIG. 15: Complementarity of Nucleotides around breakpoints is conserved across viruses. The importance of complementarity around the breakpoints was analyzed for CHIKV Caribbean strain (CHIKVc), CHIKV IOL (CHIKVi), ZIKV, EV71 and RV-B14 (RV). The y-axis indicates the frequency of complementarity, while the x-axis represents the alignment of nucleotides around the start- and stop- positions respectively. Interestingly, next to a positive signal of complementarity, the deletions coincided with a negative signal of less than 1% at the nucleotide position just prior to the breakpoint. The number of nucleotides that are complementary to each other after the breakpoint, or the primer, varies across the individual viruses. For CHIKV it is about 2 nts, 3-5 nts for ZIKV, 3-4 nts for EV71, and 2 nts for RV.

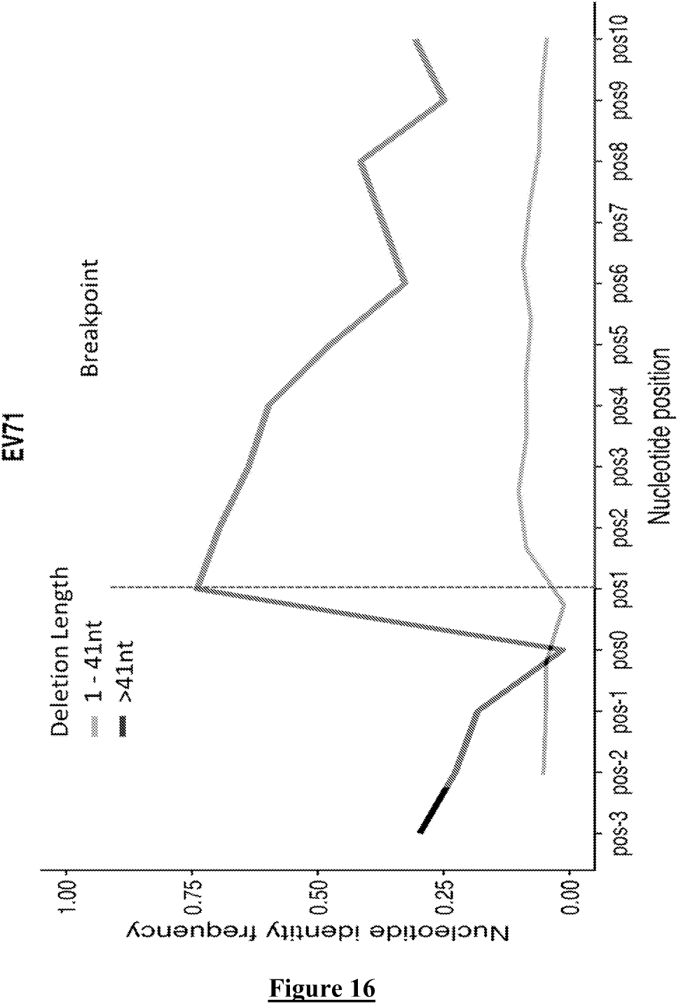

FIG. 16: The complementarity map for deletions above 42 nt show that EV71 use the “disassociate and re-prime” mechanism. There is a strong negative signal in the position just before the “primer” sequence, which is almost never identical (ca. 0.5% of the time - around 25% would be random). Then there are various primer lengths, but the average for EV71 appears to be 3 nt.

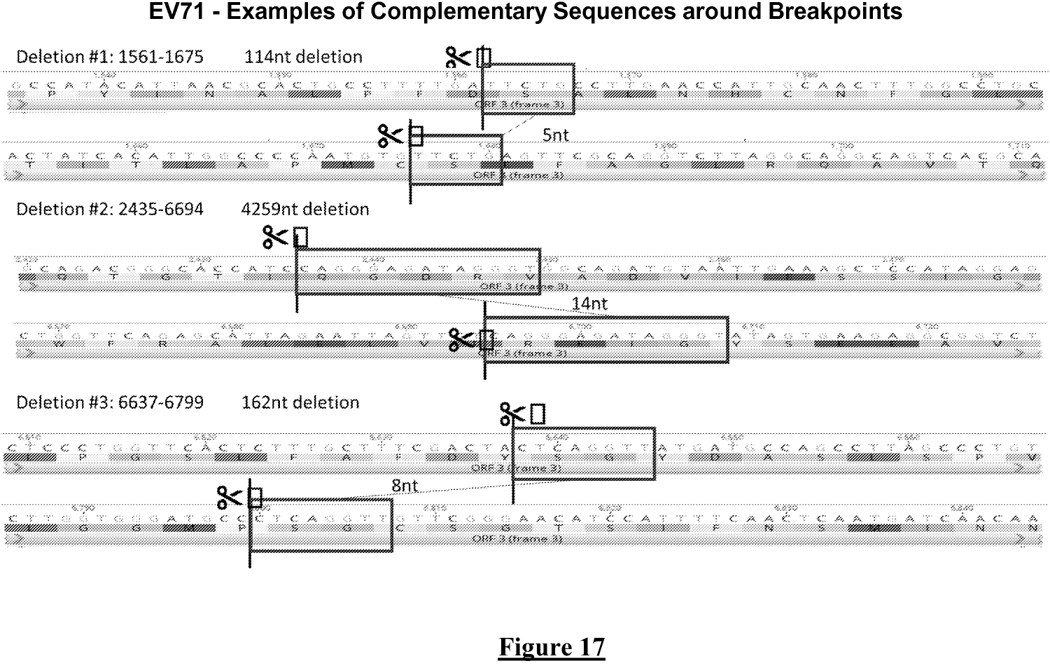

FIG. 17: Examples of Complementarity sequences around Breakpoints: Shown are three examples of DVGs that utilize complementary nucleotide sequences around the breakpoint that the polymerase can use to re-attach and re-initiate transcription, resulting in an internal deletion of the sequence between these two complementary genome regions.

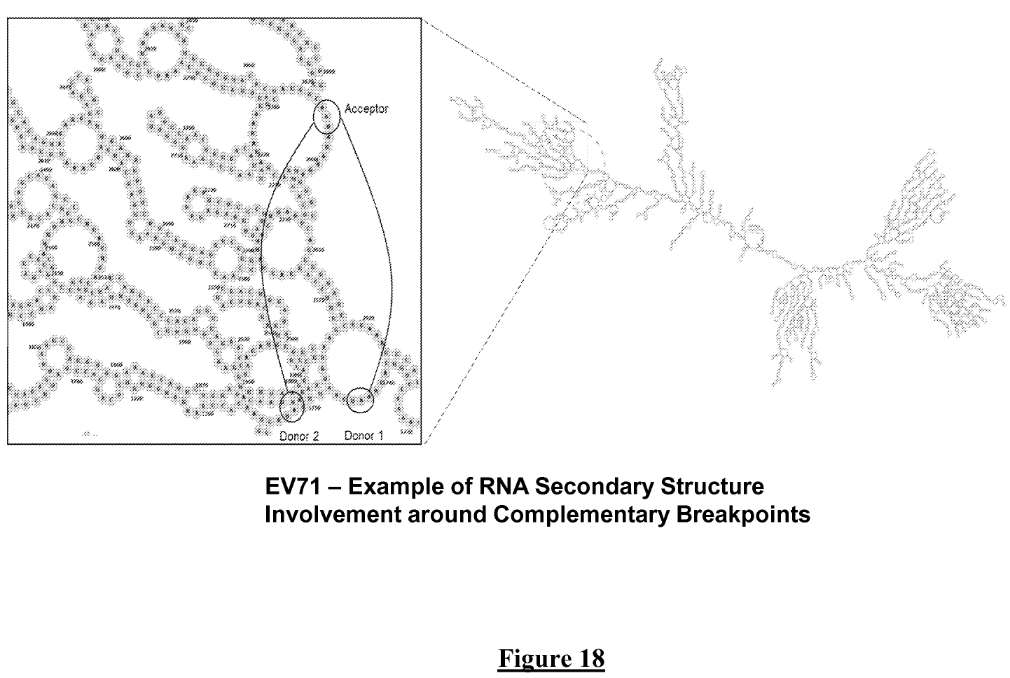

FIG. 18: Complementary Nucleotides around Breakpoints are often located in Loops in RNA Secondary Structure. The whole genome RNA secondary structure of EV71 was modelled using RNA fold (right). Deletions were mapped onto the structure and hotspots often coincided with loop structures in the secondary structure. Together with the complementary sequence around the breakpoint, this would allow these sequences to be unpaired so that the complementary sequence functions as a potential primer to bind and to allow re-initiation of transcription using a prime-realign approach. The polymerase appears to be destabilized at “donor” sides, the replication is interrupted until the polymerase can re-attach at a different location of the genome (“acceptor”) using the complementary primer and continues transcription, resulting in an internal deletion.

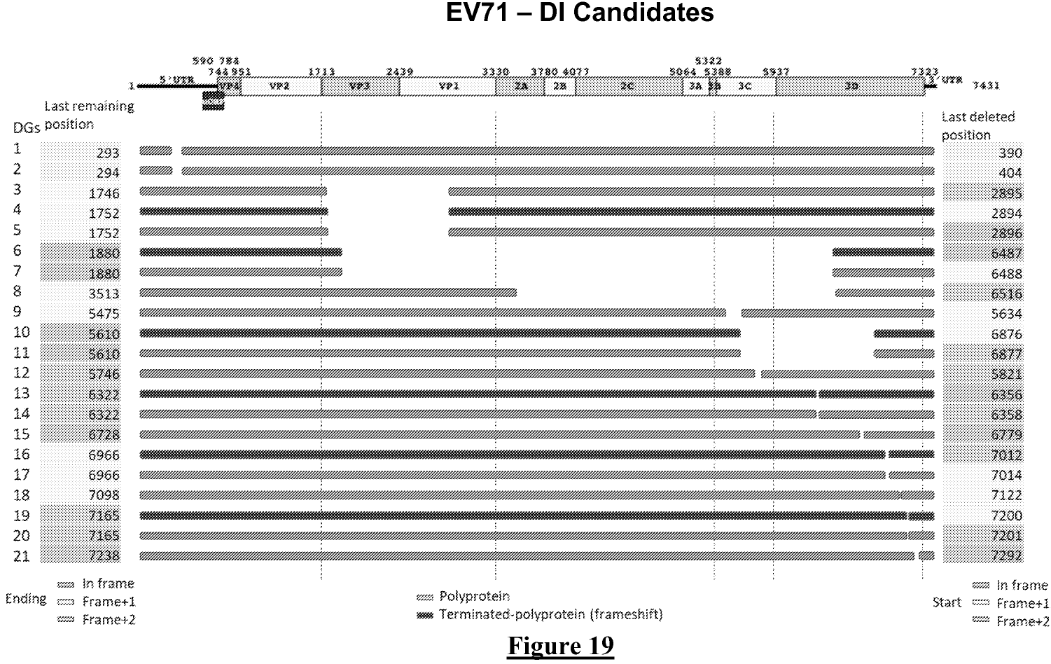

FIG. 19: EV71 DVGs sequences : SEQ ID NO: 1 (DG1 : EV71 DVG 293-390), SEQ ID NO: 2 (DG2 : EV71 DVG 294-404), SEQ ID NO: 3 (DG3 : EV71 DVG 1746-2895), SEQ ID NO: 4 (DG4 : EV71 DVG 1752-2894), SEQ ID NO: 5 (DG5 : EV71 DVG 1752-2896), SEQ ID NO: 6 (DG6 : EV71 DVG 1880-6487), SEQ ID NO: 7 (DG7 : EV71 DVG 1880-6488), SEQ ID NO: 8 (DG8 : EV71 DVG 3513-6516), SEQ ID NO: 9 (DG9 : EV71 DVG 5475-5634), SEQ ID NO: 10 (DG10: EV71 DVG 5610-6876), SEQ ID NO: 11 (DG11 : EV71 DVG 5610-6877), SEQ ID NO: 12 (DG12: EV71 DVG 5746-5821), SEQ ID NO: 13 (DG13 : EV71 DVG 6322-6356), SEQ ID NO: 14 (DG14: EV71 DVG 6322-6358), SEQ ID NO: 15 (DG15: EV71 DVG 6728-6779), SEQ ID NO: 16 (DG16: EV71 DVG 6966-7012), SEQ ID NO: 17 (DG17: EV71 DVG 6966-7014), SEQ ID NO: 18 (DG18: EV71 DVG 7098-7122), SEQ ID NO: 19 (DG19: EV71 DVG 7165-7200), SEQ ID NO: 20 (DG20: EV71 DVG 7165-7201) and SEQ ID NO: 21 (DG21: EV71 DVG 7238-7292).

The EV71 DGV sequences are listed in the following table.

| SEQ ID NO | Virus | Name | DVG size | Figure |
|---|---|---|---|---|
| 1 | EV71 | 293-390 | 7334 | FIG. 19 |
| 2 | EV71 | 294-404 | 7321 | FIG. 19 |
| 3 | EV71 | 1746-2895 | 6282 | FIG. 19 |
| 4 | EV71 | 1752-2894 | 6289 | FIG. 19 |
| 5 | EV71 | 1752-2896 | 6288 | FIG. 19 |
| 6 | EV71 | 1880-6487 | 6288 | FIG. 19 |
| 7 | EV71 | 1880-6488 | 2823 | FIG. 19 |
| 8 | EV71 | 3513-6516 | 4428 | FIG. 19 |
| 9 | EV71 | 5475-5634 | 7272 | FIG. 19 |
| 10 | EV71 | 5610-6876 | 7165 | FIG. 19 |
| 11 | EV71 | 5610-6877 | 7164 | FIG. 19 |
| 12 | EV71 | 5746-5821 | 7356 | FIG. 19 |
| 13 | EV71 | 6322-6356 | 7397 | FIG. 19 |
| 14 | EV71 | 6322-6358 | 7395 | FIG. 19 |
| 15 | EV71 | 6728-6779 | 7380 | FIG. 19 |
| 16 | EV71 | 6966-7012 | 7385 | FIG. 19 |
| 17 | EV71 | 6966-7014 | 7383 | FIG. 19 |
| 18 | EV71 | 7098-7122 | 7407 | FIG. 19 |
| 19 | EV71 | 7165-7200 | 7396 | FIG. 19 |
| 20 | EV71 | 7165-7201 | 7395 | FIG. 19 |
| 21 | EV71 | 7238-7292 | 7377 | FIG. 19 |

FIG. 20: EV71 DI candidates co-transfections

Preferred EV71 DVGs are selected from the group consisting of: SEQ ID NO: 1 (EV71 DVG 293-390,DG1), SEQ ID NO: 3 (EV71 DVG 17646-2895, DG3), SEQ ID NO: 14 (EV71 DVG 6322-6358, DG14), SEQ ID NO: 15 (EV71 DVG 6728-6779, DG15), SEQ ID NO: 18 (EV71 DVG 7098-7122, DG18), and SEQ ID NO: 21 (EV71 DVG 7238-7292, DG21).

### EV71 TIP Candidates Inhibit Wild-Type Virus In Vitro

24-wellplates with 293T cells were seeded to reach ~90% confluency the following day. 200 ng of pCAGGS-EV71_Sep006 were transfected either alone, with a control plasmid (pcDNA3-RFP) or the individual DVGs. The other plasmids were mixed with the wild-type EV71 recovery plasmid at the molarity ratios of 1:1, 5:1, or 10:1 (DVG to WT). Transfections were performed in replicates of four. The plasmids were transfected using Lipofectamine 2000 (Life Technologies), media was changed after 6 hours and samples were harvested 4 days post transfection. Virus titers were assessed either by TCID50 or by plaque assay. In brief, for TCID50 96-well plates were seeded with Vero cells and serially diluted virus supernatants, which were freeze-thawed twice, where added and samples were incubated at 37° C., and 5% CO₂ for 7 days. For plaque assays, virus was titrated in 24-well plates using VeroE6 cells that were seeded 24 h prior to infections. After 1 h incubation of the 10-fold serial diluted virus samples, the cells were covered with a semi-solid agarose overlay using 2% FBS (LifeTechnologies), MEM (LifeTechnologies), and 0.05% Agarose (Thermo). The plates were incubated at 37° C., and 5% CO₂ for 72 h. For both methods cells for fixed with 4% Formalin and plaques were visualized using crystal violet.

### Example 3: Zika Virus (ZIKV)

### Generation and Characterization of All DVGs Occurring Within Zika

Method: The virus used for the identification of DVGs is the African strain MR766 of Zika virus (ZIKV). In order to identify DVGs, ZIKV was passaged at a high multiplicity of infection in Vero, BHK or C6/36 cells. Briefly, cells that were seeded to reach between 70-80% confluence were infected with an initial MOI of 5 PFU/cell. 3 (Vero, BHK) or 5 (C6/36) days post infection, the cell culture supernatant was clarified by centrifugation, and 300 ul of the supernatant used to infect naive cells (passage 2). For low MOI conditions, 3 ul of the viral supernatant was used for infection in the subsequent passage. The passaging was repeated 12 times. RNA was extracted from the cleared supernatant (TRIzol) and used for next-generation sequencing. Libraries prepared using the RNA Library Prep kit (Illumina) were loaded on an Illumina NextSeq500 sequencer, using 150 cycle single-end reads. The reads obtained were analyzed using the computational pipeline described above.

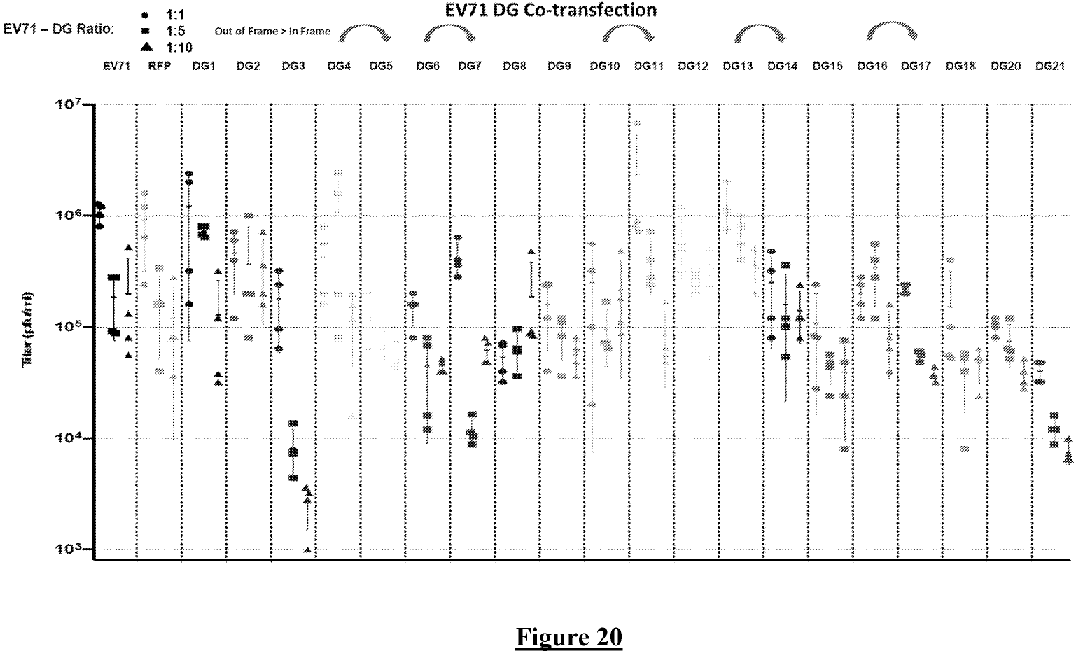

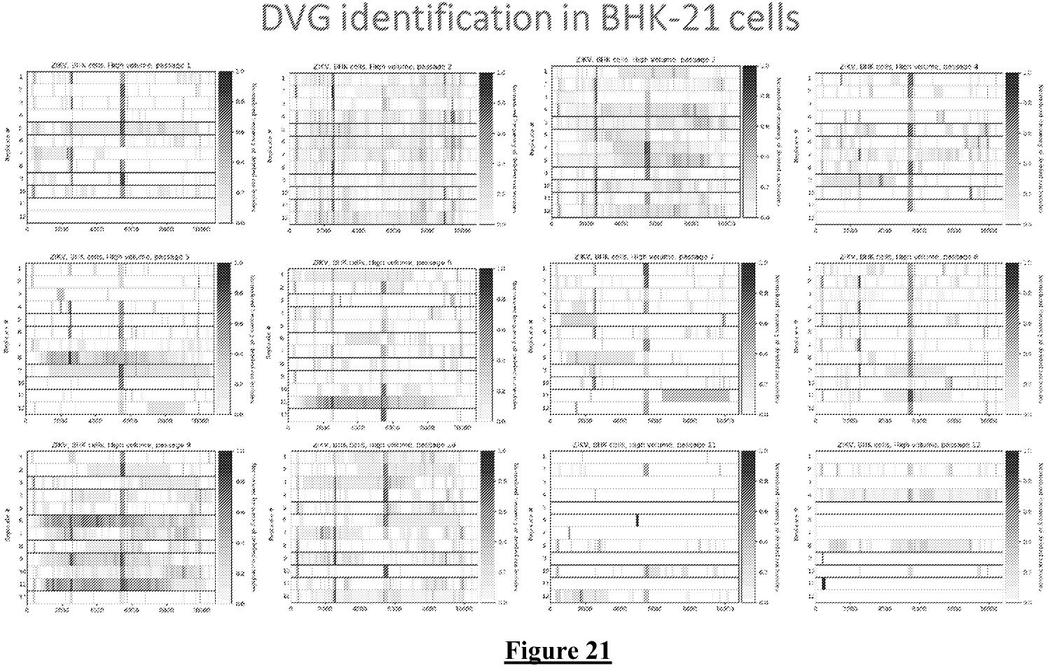

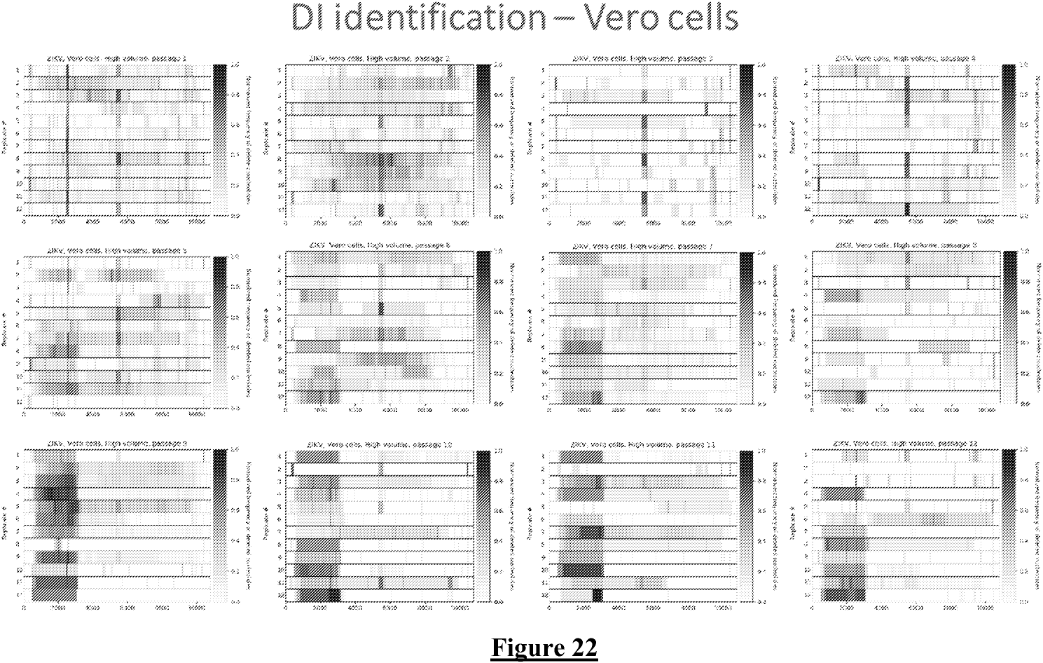

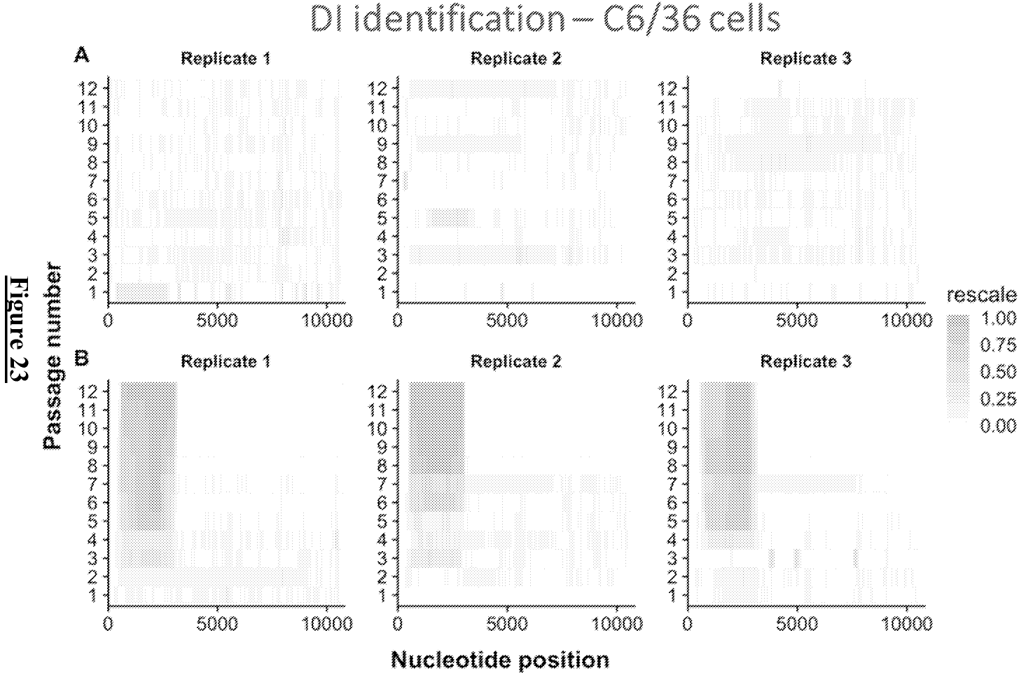

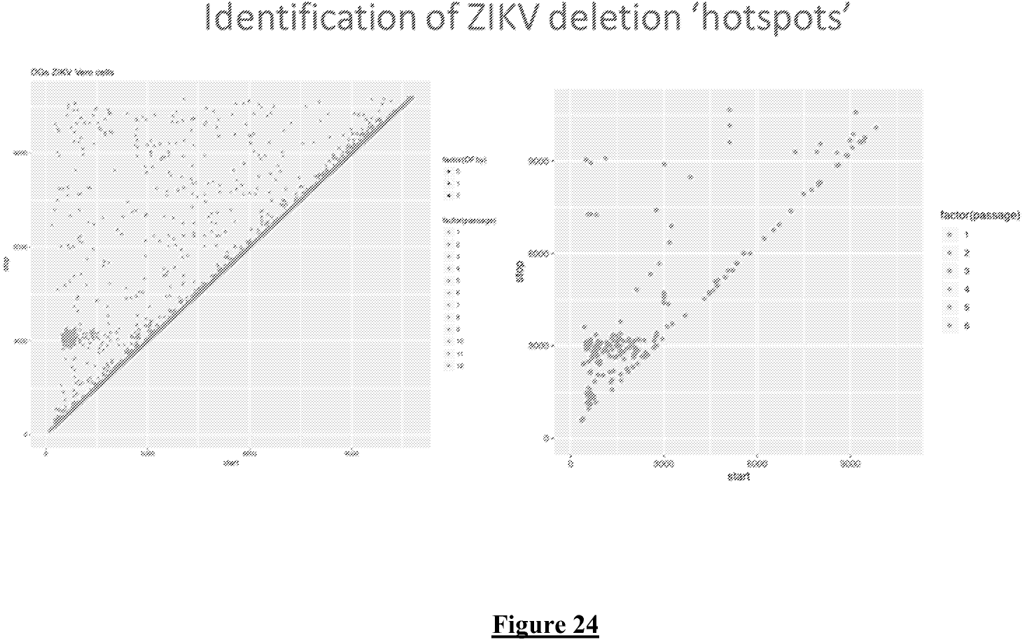

Deep sequencing of samples and computational analysis identified all deletion hotspots across the genome and specific DVGs occurring within the population. In FIG. 21, each panel represents a different passage number, while each row in each panel is a different biological replicate, performed in BHK cells. Analysis reveals deletion hotspots that are common to several replicates, suggesting these DVGs are of higher fitness than the many others that carry random deletions. Deletions also occur across passages, suggesting that the corresponding DVGs are higher fitness and can be packaged to infect new cells. In FIG. 22, the same analysis was performed for virus passaged in Vero cells. The data reveal that while in initial passages there are some deletions and DVGs that are also found in BHK cells, certain deletion hotspots (in the 5’end of th genome) are unique to this cell line and take over other deletions in terms of frequency. In FIG. 21, the deletion hotspots show deleted regions in 5′ end of the ZIKV genome which is similar to the pattern observed in Vero cells. Altogether, the data show the importance of using more than one cell line or host to determine the total breadth and overall fitness of DVGs in different conditions. A further comparison of the common and unique DVGs found in different cell types is shown in FIG. 24, where Zika grown in Vero (left panel) and C6/36 (right panel) are compared. The figure highlights that there are many differences between cell types, yet also reveals a cluster of deletions in both cell types that emerges as the passage series progresses (suggesting high fitness of these DVGs).

From these and other analyses, we selected a list of TIP candidates following our criteria for selection. The candidates were named A, B, C, D, E, F, G, H, K and L. The candidates span a variety of sizes and the deletions occur in different places in the genome, representing a wider array of TIP candidates than could be identified using conventional passaging and isolation.

FIG. 21. DVG identification in BHK-21 cells. ZIKV populations from each passage were deep sequenced (RNAseq) and the normalized frequency of each deleted nucleotide position (x axis) throughout the ZIKV genome is shown as a heat map. Each panel represents a different passage number for all high MOI replicates. Each row within each panel represents each of 12 biological replicates.

FIG. 22: DVG identification in Vero cells. ZIKV populations from each passage were deep sequenced (RNAseq) and the normalized frequency of each deleted nucleotide position throughout the ZIKV genome is shown as a heat map. Each panel represents a different passage number for all high MOI replicates.

FIG. 23: DVG identification in C6/36 cells. ZIKV populations from each passage were deep sequenced (RNAseq) and the normalized frequency of each deleted nucleotide position throughout the ZIKV genome is shown as a heat map. Each panel represents a different passage number for all high MOI replicates.

FIG. 24: Identification of ZIKV DVG hotspots. Start (x axis) and stop (y axis) positions of the deletions were plotted for Vero (A) and C6/36 (B) cell passages. Data points are colored according to passage number. A clear hotspot was observed in the region with start and stop positions at approximately 500-3000 nt start-stop, particularly in Vero cells

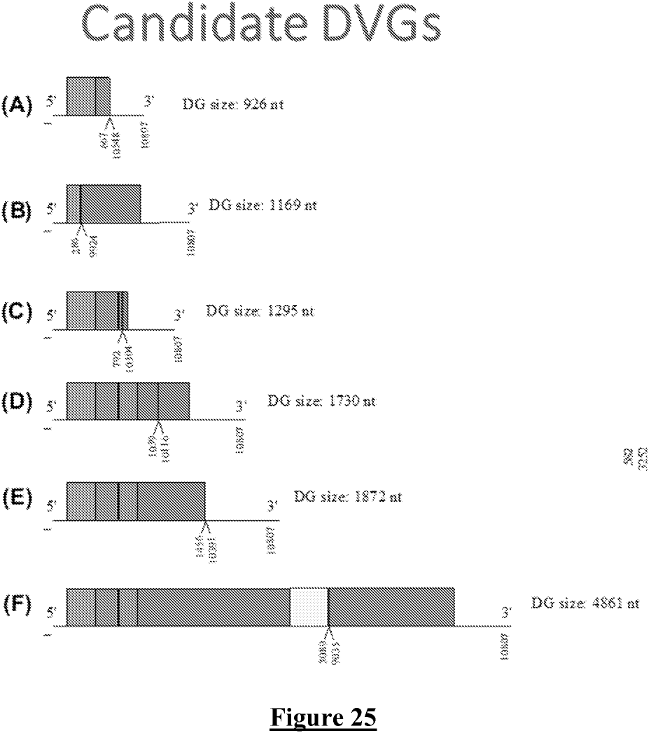

FIG. 25: Schematic representation of candidate ZIKV DVGs. ZIKV candidate DVGs were chosen based on their redundancy and frequency throughout passaging. Each letter on the left represents the provisional name of the DVG.

### Zika TIP Candidate K Is Non-Replicative Per Se

Method: DG K-based replicon constructs were designed to assess the ability of the DG to replicate. Nanoluc reporter constructs were generated in which the reporter is inserted in place of the structural proteins, as is normally designed for flavivirus replicons (FIG. 29 A). RNA was generated from the DNA clones by in vitro transcription, and transfected into Vero cells. At 6, 24, 48 and 72 h post transfection, nanoluc reporter activity was measured in cells.

The results show that unlike the WT replicon for which an increase in reporter activity is observed over the time course, the DG K replicon showed no increase (FIG. 29 B). However, when the assay was repeated in infected cells, replicon activity is recovered (FIG. 29 C), suggesting that the DG is non-replicative by itself, but requires WT for its replication. These results have important implications in terms of safety of the use of DVGs as potential TIPs.

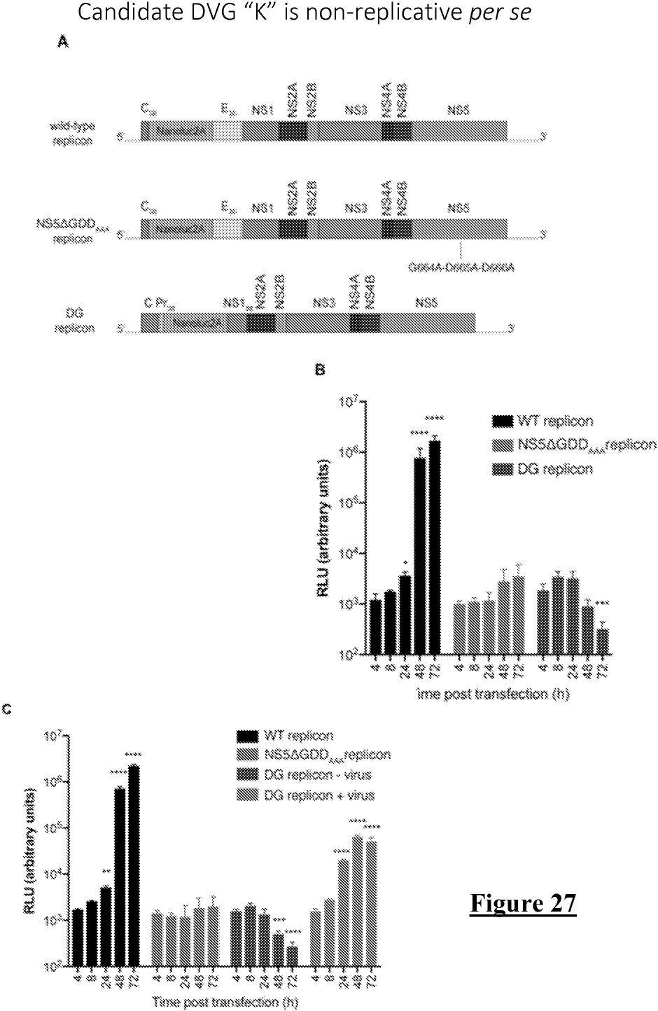

FIG. 27. ZIKV DG is non-replicative per se and depends on WT for its replication. (A) Schematic diagram of replicon constructs. For the wild-type replicon, C₃₈ and E₃₀ represent the N-terminal 38 aminoacids of C and the C- terminal 30 aminoacids E proteins, respectively. For the DG replicon, Pr₃₈ and NS1₉₈ represent the N-terminal 38 aminoacids and the C- terminal 98 aminoacids of Pr and NS1 proteins, respectively. Nanoluc2A represents the nanoluc reporter sequence followed by the foot-and-mouse disease virus 2A protease. An inactive mutant replicon (NS5ΔGDD_{AAA} replicon) was also constructed. (B) Wild-type and DG replicon assays. Equal amounts of wild-type, NS5ΔGDD_{AAA}, and DG replicon RNA were transfected in Vero cells, and relative light units (RLU) measured at the indicated times post-transfection. (C) DG replicon assays in infected or non-infected cells. Equal amounts of DG replicon RNA was transfected in naive or infected Vero cells (MOI 1 PFU/cell), and relative light units (RLU) measured at the indicated times post-transfection. Wildtype and NS5ΔGDD_{AAA} replicons were carried out as controls. All graphs show the mean and SD; n=3 per group for a representative experiment, and the time points within each group were compared to RLU values at 4 h. Two-way ANOVA with Dunnet’s multiple comparison test (^{∗} = p≤ 0.05, ^{∗∗}= p≤ 0.01, ^{∗∗∗}= p≤ 0.001, ^{∗∗∗∗}= p≤ 0.0001) was performed.

### Zika TIP Candidates Inhibit Wild-Type Virus In Vitro

Method: HEK293T cells were transfected with a plasmid encoding wild-type ZIKV or the candidate DVGs in increasing molar ratios (1:10, 1:1 or 10:1 DVG:WT ratio). 4 days post transfection, virus titer was determined by plaque assay.

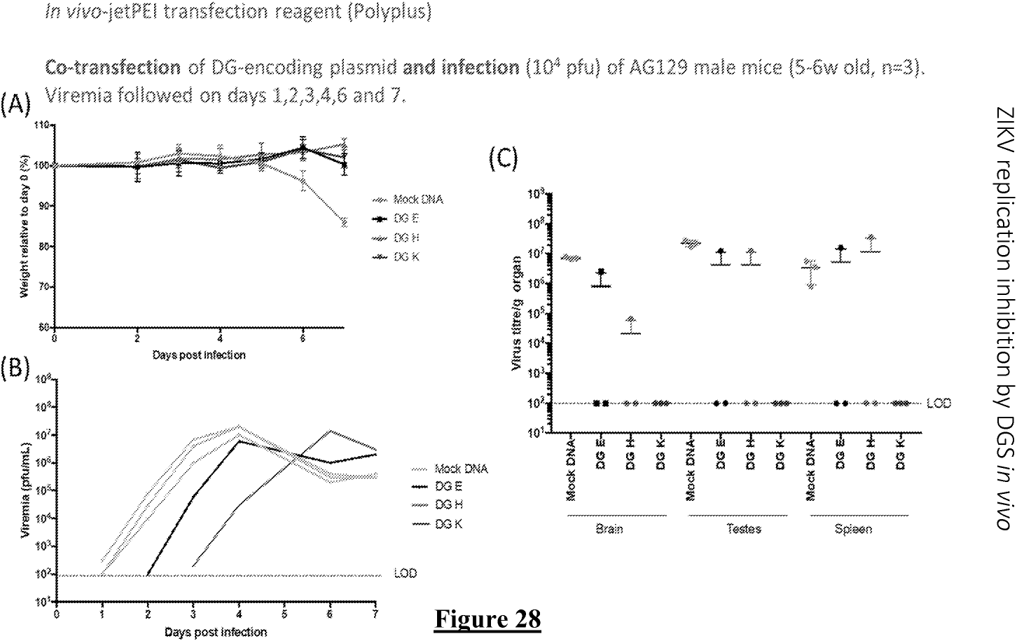

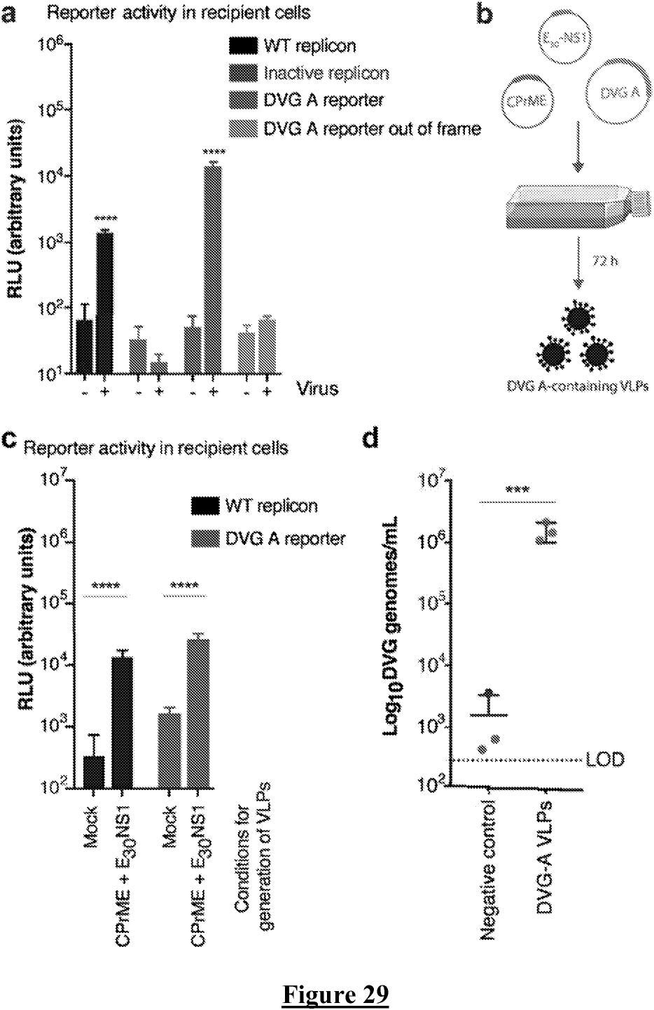

The candidate TIPs identified above were tested in vitro for their ability to inhibit wild-type Zika virus. Cells were transfected with a plasmid coding for Zika virus, along with plasmids coding for each DVG at increasing molar ratios. The effect of the TIP candidate on WT virus growth was determined by measuring the amount of progeny virus after treatment. The data (FIG. 28) show that at highest ratios, all TIP candidates except DG-G or the out-of-frame DG-H had at least a slight inhibitory effect; while DG-C, -D, -E, -F, -K and -L lowered viral titers by 1 log or more. The results also reveal a dose dependence of inhibition.

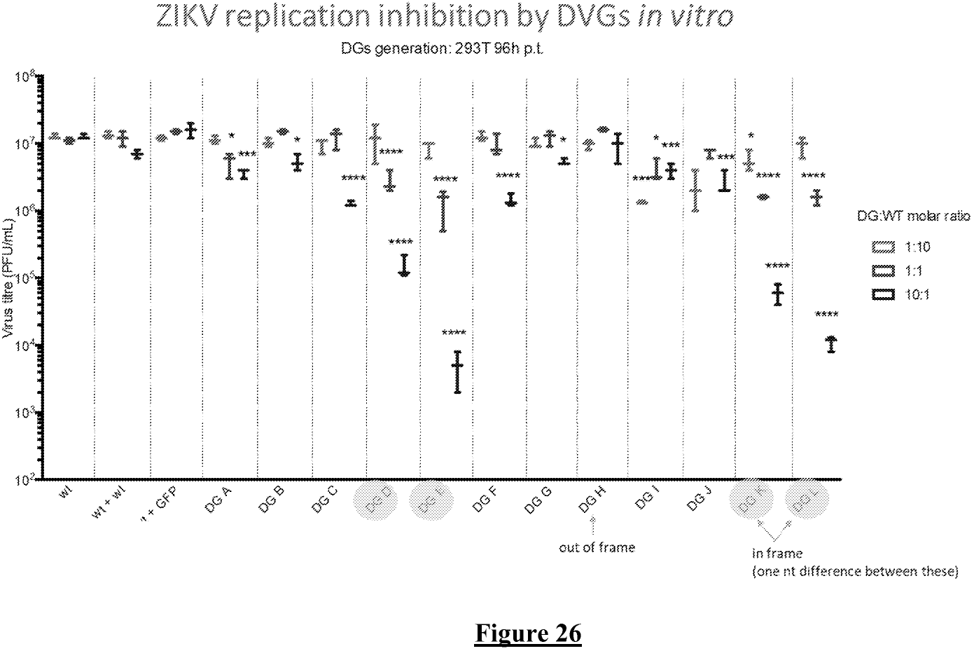

FIG. 26: Candidate DVG inhibition of ZIKV replication in vitro. HEK293T cells were transfected with plasmids encoding ZIKV candidate DVGs and wild-type encoding plasmid at increasing molar ratios (1:10, 1:1 or 10:1 DVG:WT). 4 days post-transfection, the cell culture supernatant was harvested and ZIKV titers determined by plaque assay to determine whether the candidate DVGs have an interfering effect. Two-way ANOVA with Dunnet’s multiple comparison test (^{∗} = p≤ 0.05, ^{∗∗}= p≤ 0.01, ^{∗∗∗}= p≤ 0.001, ^{∗∗∗∗}= p≤ 0.0001) was carried out. Each condition was compared to cells transfected only with wild-type virus.

### Zika TIP Candidates Inhibit Wildtype Virus In Vivo

Method in mice: AG129 male mice (4-6 week old) were infected with 10⁴ PFU of Zika virus and co-transfected with 20 ug plasmid encoding DG-E, —H or -K (candidate DVGs that exhibited the highest inhibition effect in vitro), using the in vivojetPEI transfection reagent (Polyplus), in a total volume of 50 ul via a footpad injection (s.c.). Control mice received a control plasmid not coding for any TIP. The ability of TIPs to attenuate infection was monitored by weight loss. Viremia was also measured daily (except day 5). 7 days post infection, mice were euthanized and spleen, brain and tested dissected for determination of virus titer in these organs.

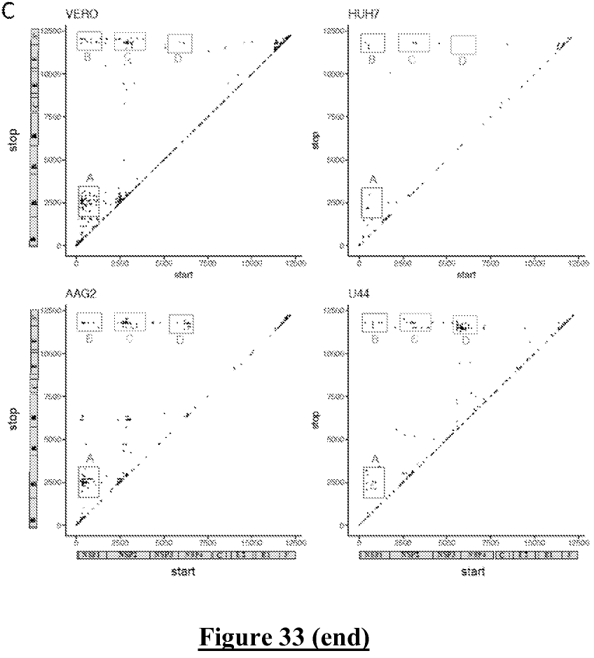

The results show (FIG. 28A) that all three TIPs protected mice from weight loss, while those that receiving the control plasmid had significant weight loss by day 7. We monitored the effect of TIP treatment on viremia (FIG. 28B). The data reveal that all three mice receiving control plasmid had high levels of virus in the blood as early as day 2 after infection. 2 of 3 mice receiving either DG-E or DG-H had no viremia, while one mouse from each group had a 1-2 day delay in viremia. No mice receiving DG-K had any viremia. We then measured the virus titers in the brains, testes and spleens of mice on day 7 (FIG. 28C). All mice receiving the control treatment had very high titers in each organ. The mice that presented delayed viremia in FIG. 33B, showed reduced titers in the brain, and similar titers in other organs. All other mice were fully protected with no detectable virus in any tissue.

FIG. 28: ZIKV candidate DVGs -E, -H and -K protect AG129 mice from ZIKV infection. AG129 mice (male, 5-6 weeks) were injected a mix of 20 ug of DVG-encoding plasmid (or a mock plasmid control) in a transfection mix and 10⁴ PFU of ZIKV. (A) Weight loss was monitored daily. Only mice transfected with mock plasmid lost weight by 7 days. (B) Viremia was followed daily. While mice transfected with mock plasmid exhibited a similar viremic profiles, only one out of three mice showed delayed viremia for DVG-E and DVG-H. All other mice (including those transfected with DG K) did not display viremia at any time point. (C) 7 days p.i. mice were euthanized and brain, testes and spleen collected. Infectious virus was only detected in mice that displayed viremia.

Method of VLP packaging : Uninfected or infected Vero cells (producer cells, MOI 1 PFU/cell at 24 h) in 24-well plates were transfected with 100 ng of DVG replicon RNA, WT replicon (positive control) or inactive replicon (negative control), using TransIT-mRNA transfection reagent (Mirus Bio). 48 h p.t. the supernatant endonuclease-treated (25 Units/µl BaseMuncher, Expedeon) and used to infect naive Vero cells in 24-well plates (recipient cells). Replicon activity in recipient cells was assessed 24 h p.i. Donor and recipient cells were lysed in passive lysis buffer (Promega) and luciferase activity measured using the Nano-Glo luciferase assay system (Promega) in a Tecan infinite M200 pro plate reader (Tecan group). HEK-293T cells seeded in a 24 well plate were transfected with a mix of 200 ng of a CPrME-encoding plasmid, 200 ng of E₃₀-NS1, and 200 ng of the DVG-encoding plasmid (LT-1 transfection reagent, Mirus Bio). 72 h p.t., the medium was clarified by centrifugation. N naive recipient cells were infected with producer cell supernatant following endonuclease (Basemuncher, Expedeon) treatment, and successful packaging determined with luciferase measurement when using reporter DVG clones, or by RT-qPCR when using native DVG.

Method of VLP-DVGs in mice: 4-6 week old AG129 or C57BL/6 female mice were given a mix of Ketamine (10 mg/mL)/Xylazine (1 mg/mL) by intraperitoneal injection. Once anesthetized, mice were inoculated by a subcutaneous (footpad) route with vehicle (DMEM media), 10⁴ PFU of Zika virus alone, or complemented with DVG-containing VLPs (TIPs). C57BL/6 mice were treated with 2 mg of an IFNAR1 blocking mouse MAb (MAR-5A3, Euromedex) by intraperitoneal injection one day prior to infection. Weight loss and viremia were monitored at the indicated times after infection. Blood was collected from the facial vein in Microtainer blood collection tubes (BD) and allowed to clot at room temperature. Serum was separated by centrifugation and stored at -80° C. Viremia was quantified either by plaque assay or RT-qPCR on unextracted and diluted serum samples, as described previously¹⁹. Infected mice were euthanized by cervical dislocation. Spleen, ovaries, brain and injected footpads were harvested and homogenized with 600 µl DMEM supplemented with 2% FBS in Precellys tubes containing ceramic beads, using a Precellys 24 homogenizer (Bertin Technologies) at 5000 rpm for 2 cycles of 20 seconds. Homogenates were cleared by centrifugation, and the supernatant stored at -80° C. until virus titration or RT-qPCR. Viral burden in organs was measured either by plaque assay (expressed as PFU/g for organs) or by RT-qPCR (expressed as PFU equivalents relative to GAPDH) following RNA extraction using Direct-zol-96 (Zymo). The number of DVG or WT RNA genomes in mouse organs was normalized to GAPDH genomes, derived from a standard curve using mouse GAPDH primers (Supplementary Table 1) and RNA extracted from the respective organ of uninfected mice.

FIG. 29. Generation of DVG-A-carrying virus-like particles. (a) Assessment of DVG-A packaging ability by WT virus. Infected or uninfected cells were transfected with WT replicon, inactive replicon, DVG-A reporter, or DVG-A out-of-frame reporter RNA. 48 h post-transfection, the cell supernatant was collected from donor cells, depleted of naked RNA, and used to infect naive recipient cells, which were harvested 24 h later for measuring reporter activity. Luciferase activity in recipient cells is shown. ^{∗∗∗∗}= p≤ 0.0001 (by two-way ANOVA with Dunnet’s multiple comparison, as compared to uninfected cells) (b) Schematic illustration of VLP assays: HEK-293T cells were transfected with a CPrME, E30-NS1 and DVG -encoding plasmids. 72 h post-transfection the cell culture supernatant with DVG containing VLPs was harvested. (c) VLP assays using WT replicon and DVG-A reporter. Donor cells were transfected with WT replicon or DVG-A reporter plasmid, with or without (mock) CPrME and E₃₀-NS1. The cell culture supernatant was treated with nuclease and used to infect naive recipient cells. Reporter activity in recipient cells is shown. ^{∗∗∗∗}= p≤ 0.0001 (by two-way ANOVA with Dunnet’s multiple comparison, as compared to mock conditions). (d) Quantification of DVG-A genomes in VLPs generated using native DVG-A. Clarified and nuclease-treated supernatant from donor cells were subjected to DVG-A quantification using RT893 qPCR. Negative control refers to supernatant derived from cells transfected with the same amount of DVG-A-containing plasmid in the absence of CPrME or E₃₀-NS1. ^{∗∗∗}: p= 0.0004 (by two-tailed t-test). All graphs show the mean and SD; n=3 per group of a representative experiment. of three.

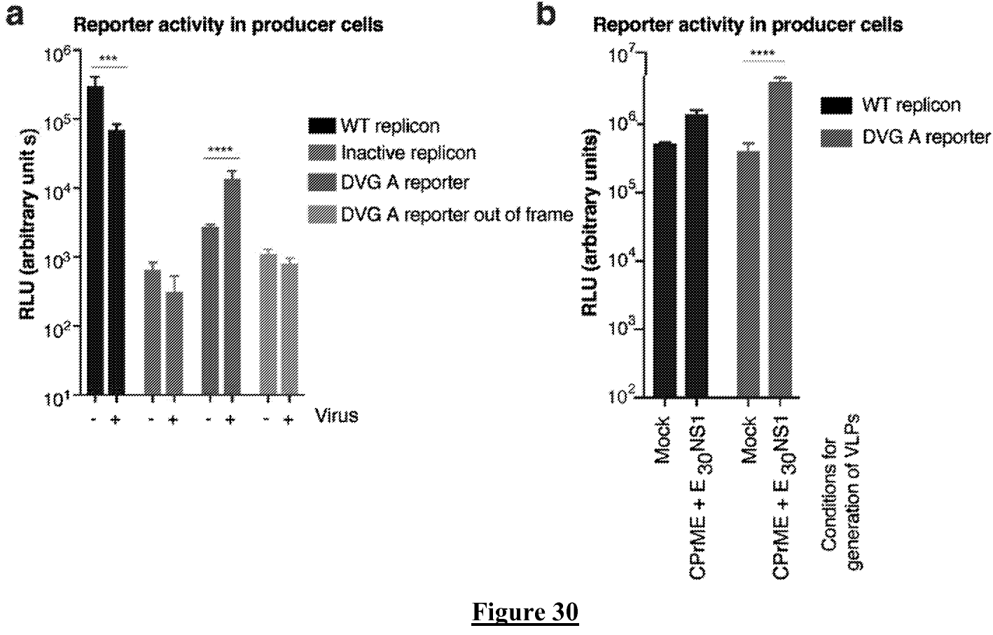

FIG. 30. Reporter activity in producer cells of packaging assays. (a) Infected or uninfected Vero cells were transfected with WT replicon, inactive replicon, DVG-A reporter or DVG-A out-of-frame reporter RNA. 48 h post transfection, the cell supernatant was collected from producer cells, depleted of naked RNA, and used to infect naive recipient cells (shown in FIG. 4a). Reporter activity in producer cells is shown. ^{∗∗∗}: p= 0.0004, ^{∗∗∗∗}= p≤ 0.0001 (by two-1011 way ANOVA with Dunnet’s multiple comparison, as compared to uninfected cells). (b) VLP assays using WT and DVG-A reporter. Donor cells were transfected with WT or DVG-A reporter with or without (mock) CPrME and E₃₀-NS1. The cell culture supernatant was treated with nuclease and used to infect naive cells. Replicon activity (72 h p.t.) in producer cells is shown. ^{∗∗∗∗}= p≤ 0.0001 (by two-way ANOVA with Dunnet’s multiple comparison, as compared to mock conditions). All graphs show the mean and SD; n=3 per group for a representative experiment of three.

DVG-A can be packaged into virus-like particles (VLPs) for in vivo delivery. To consider DVGs as a potential therapeutic agent, we may require an appropriate delivery method. Thus, we established a packaging system to encapsidate a DVG into virus-like particles (VLPs). We first had to confirm that this DVG could be encapsidated at all. To this end, we transfected uninfected or infected cells with DVG-A reporter or control WT replicon RNA. These cells, named ‘producer’ cells, should (if infected) produce WT virus progeny, as well as virions containing genomes that can be packaged. Transfer of the supernatant derived from infected producer cells to naive (recipient) cells enabled us to assess packaging of reporter-expressing genomes. Significantly higher WT replicon and DVG-A reporter activities were observed in recipient cells in the presence of WT virus, compared to background activity in the absence of virus, confirming that DVG-A was packaged by WT virus (FIG. 29a, producer cell reporter activity is shown in FIG. 30a). Comparatively, no increase in reporter activity was detected for the inactive replicon or DVG-A_out-of-frame, suggesting that replication is a requirement for genome packaging. Next we developed a VLP-producing system to encapsidate DVG-A in the absence of WT virus. Briefly, we transfected cells with plasmids encoding DVG-A, the structural proteins (CPrME) and the non-structural protein NS1, to enable packaging and replication of DVG-A, respectively (FIG. 29b). Packaging of DVG-A into VLPs was then assessed following transfer of supernatants onto naive recipient cells. Using reporter DVG-A, we confirmed that the reporter activity in recipient cells for packaged DVG-A was comparable to that of packaged WT replicon (FIG. 4c, producer cell reporter activity shown in FIG. 30b). Finally, we confirmed that native DVG-A was packaged into VLPs, as shown by RT-qPCR of transfected cell supernatants (FIG. 29d). Thus, DVG-A can be actively packaged into VLPs, which could serve as therapeutic interfering particles (TIPs) for in vivo evaluation.

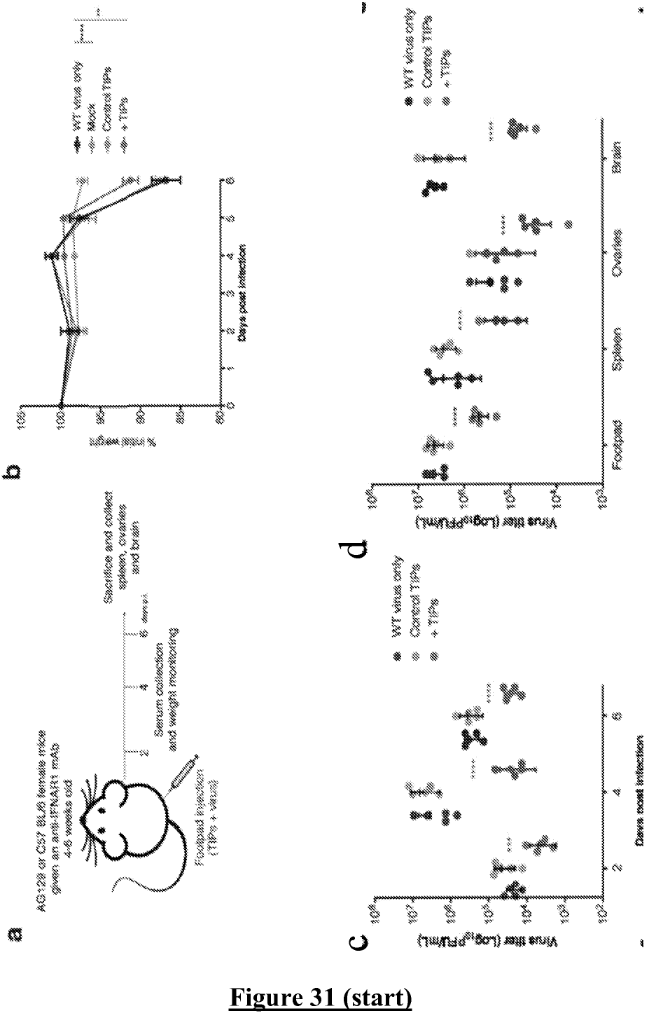

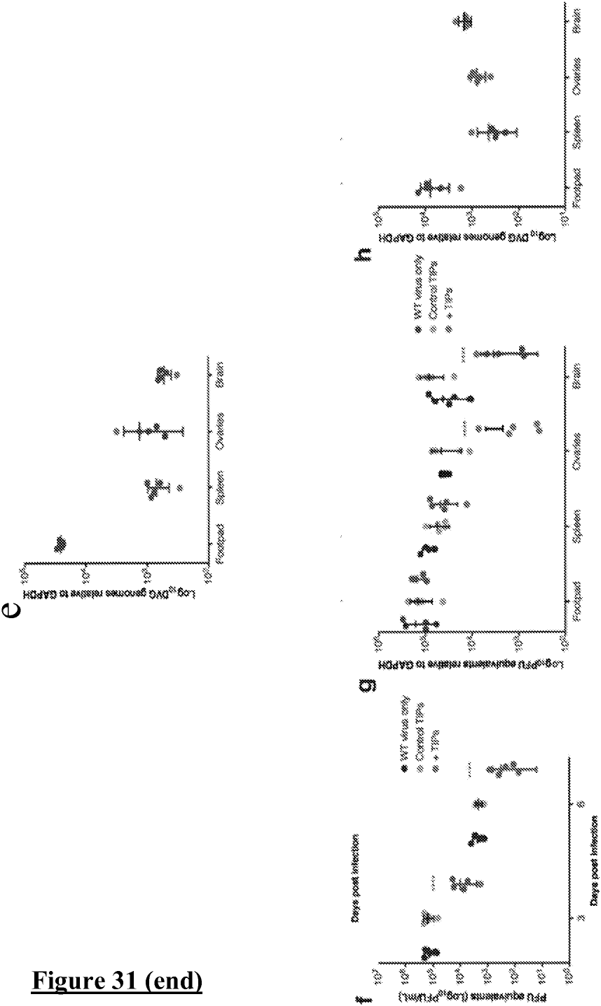

FIG. 31. Zika virus DVG-A-carrying TIPs inhibit virus replication in mice. (a) Experimental design. 4-6 week old AG129 or C57BL/6 female mice (n=5) were inoculated with WT virus diluted in DMEM, mock TIP supernatant (from transfections performed with no structural proteins), or with supernatant confirmed to contain DVG-packaging VLPs (TIPs). C57BL/6 mice were treated with 2 mg of IFNAR1-blocking monoclonal antibody 24h prior to infection. At different times post-infection, sera were collected. 6 days p.i. mice were euthanized and organs harvested. (b-e) AG129 mice: (b) Weight loss of infected mice is shown as a % of weight at day 0. ^{∗∗}:p= 0.011; ^{∗∗∗∗}: p≤ 0.0001. (c) Viremia on days 2, 4 and 6 post infection. ^{∗∗∗}: p= 0.0007; ^{∗∗∗∗}= p≤ 0.0001. (d) Virus load in the injected footpad, spleen, ovaries and brain, 6 days p.i. ^{∗∗∗∗}= p≤ 0.0001. (e) DVG amounts in all organs collected measured by RT-qPCR. (f-h) C57BL/6 female mice that were given an IFNAR1-blocking monoclonal antibody 24 h prior to infection. (f) 3 and 6 days p.i. viremia was measured by RT-qPCR and is depicted as PFU equivalents/mL. ^{∗∗∗∗}= p≤ 0.0001. (g) 6 days p.i. mice were euthanized and virus load in organs measured by RT-qPCR. ^{∗∗∗∗}= p≤ 0.0001. (h) DVG-A amounts in all organs measured by RT qPCR. In all cases, two-way ANOVA with Dunnet’s multiple comparison was carried out comparing each condition to mice infected with WT virus only.

Zika virus VLP-DVGs reduce infection and virulence in mice. We next assessed the efficacy of these DVGs packaged as VLPs as an antiviral measure in mice. We used mice deficient in α/β and γ receptors (AG129 mice), as these are highly susceptible to Zika virus infection and disease progression. Mice were mock-infected (vehicle alone), infected with 10⁴ PFU of WT virus alone, or with a mixture of WT virus and DVG-A. Approximately 10 DVG-A genome copies per Zika virus infectious virion were used. As an additional control, mice received a mix of WT virus and supernatant from mock conditions of production (‘control TIPs’, generated in the absence of CPrME or NS1) (FIG. 31a). All mice lost weight by day 6, except mock-infected mice. At this time, TIP-treated (but not control TIP-treated) mice presented significantly lower weight loss than WT virus-infected mice (FIG. 31b). Further, unlike mice receiving WT virus alone or control treatment, TIP-treated mice presented significantly lower viremia at 2, 4 and 6 days p.i., with up to 2-log differences in virus titer (FIG. 31c). Viral loads in the footpads, spleens, ovaries and brains were also 1-2 log lower in TIP-treated mice on day 6, underscoring the protective effect of this TIP (FIG. 31d). While DVG-A RNA in circulating blood was below detection levels, we confirmed the presence of DVG-A RNA in the footpads of TIP-treated mice, and at lower quantities in the spleens, ovaries and brains (FIG. 31e). These results confirm that DVG-A TIPs successfully disseminate from the injection site to distal organs. In further support, we assessed TIP efficacy in a more immunologically competent mouse model. C57BL/6 mice were treated with IFNAR1-blocking monoclonal antibody before infection with Zika virus, with or without TIPs. Weight loss was not monitored, as mice do not lose weight nor succumb to infection under these conditions. Viremia was significantly lower in DVG-treated mice at 3 and 6 days p.i. (FIG. 31f). While we did not observe differences in viral loads in the footpads or spleens, viral loads in ovaries and brains were significantly lower in TIP-treated mice compared to control conditions, with up to 2-log difference in viral loads (FIG. 31g). As noted in AG129 mice, the high quantities of DVG-A at the injection site disseminated to the spleens, brains, and ovaries of TIP-treated mice (FIG. 31h).

Method in mosquitoes: Ae. aegypti mosquitoes were injected with 0.02pmoles RNA using Cellfectin transfection reagent (control RNA, DG H or DG K). 2 days post injection, mosquitoes were fed a bloodmeal containing 7 ₓ10⁵ pfu/mL ZIKV The ability of TIPs to attenuate infection was monitored by dissection of mosquito body parts at 8 or 13 days post infection and determination of viral load. At 13 d p.i. mosquitoes were also salivated to investigate whether transmission could be blocked in the presence of TIPs.

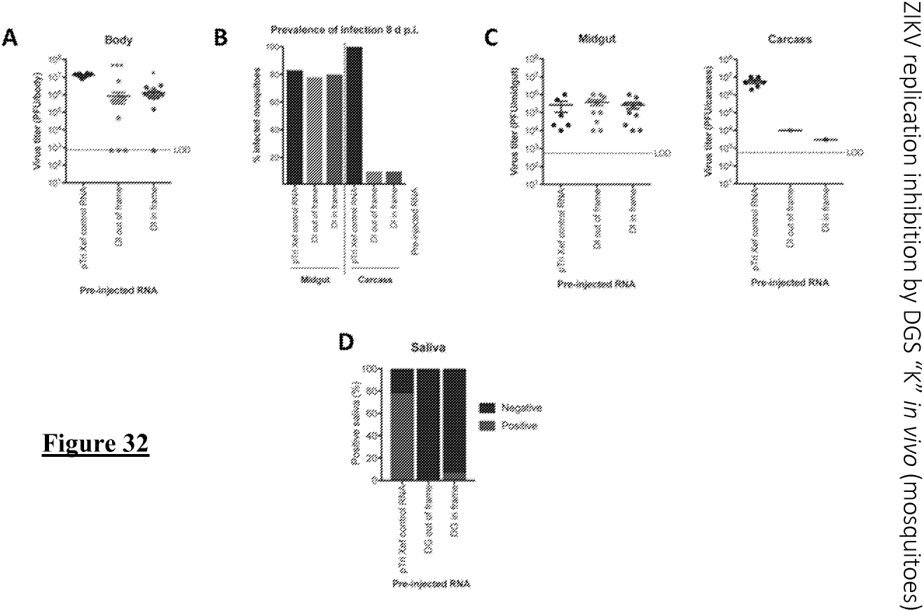

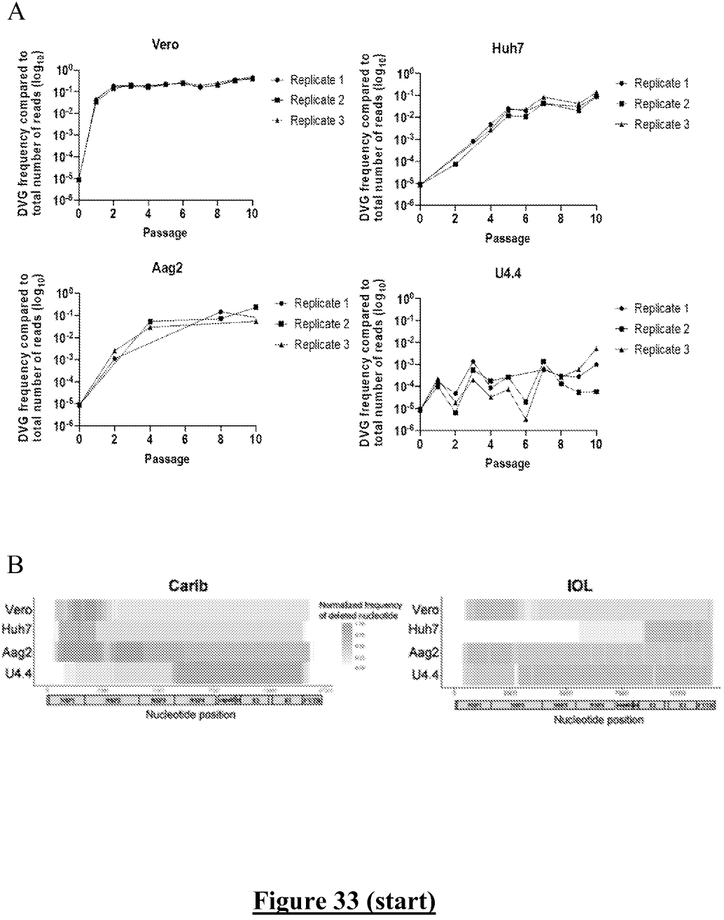

The results show that, in whole bodies of mosquitoes at 13 days p.i. there was a significant decrease in viral load in mosquitoes injected with DG H and DG K, suggesting virus attenuation in the mosquitoes in the presence of TIPs (FIG. 32 A). While at 8 d p.i. overall infection rates were not affected in control, DG H or DG K-injected mosquitoes, prevalence in the carcass was significantly affected (FIG. 32 B), suggesting that DGs attenuate virus dissemination in mosquitoes. Furthermore, while midgut titers were similar for all conditions, the only positive mosquitoes which were positive in the carcass reached lower titers than in control RNA-injected mosquitoes (FIG. 32 C). Finally, while at 13 d p.i. 7 out of 9 control RNA-injected mosquitoes were ZIKV positive in the saliva (and thus could potentially transmit the virus), no saliva was positive for DG H (n=13) and one out of 16 mosquitoes was positive for DG K injected mosquitoes (FIG. 32 D).

FIG. 32: ZIKV DG H and K inhibits virus replication, dissemination and transmission in experimentally-infected mosquitoes. Ae. aegypti mosquitoes were injected with 0.02pmoles RNA. 2 days post injection, mosquitoes were fed a bloodmeal containing 7 ₓ10⁵ pfu/mL ZIKV, and dissected at 8 or 13 d p.i. (A) Viral load in whole bodies of mosquitoes pre-injected with control (n=8), DG out of frame (n=11) or DG in frame (n=10) RNA, 13 d p.i. (B) Prevalence of ZIKV infection in the midgut or carcass of mosquitoes pre-injected with control (n=6), DG out of frame (n=10) or DG in frame (n=10), 8 d p.i. (C) Viral load in the midgut or carcass of the same mosquitoes from (B). (D) Percentage ZIKV-positive saliva in mosquitoes pre-injected with control (n=9), DG out of frame (n=13) or DG in frame (n=16), 13 d p.i. DG out of frame = DG H; DG in frame= DG K.

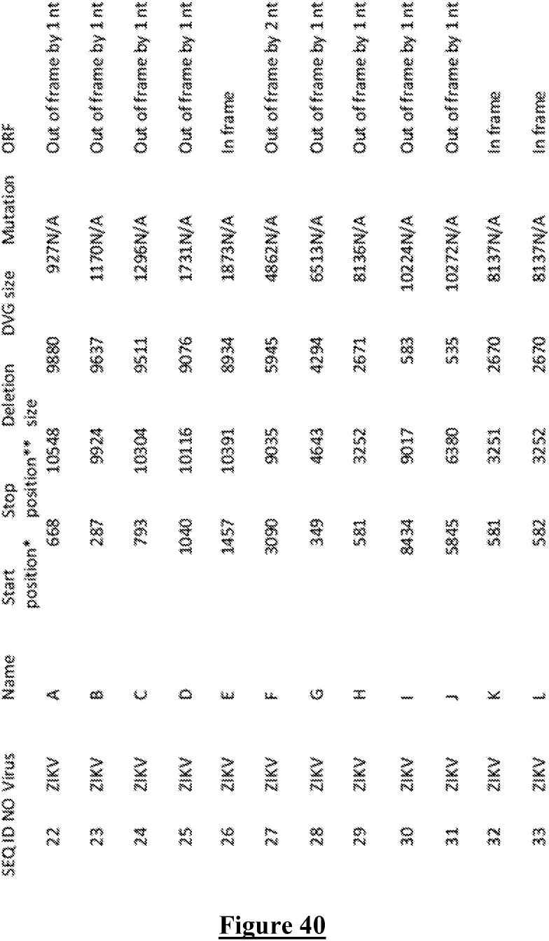

The identified ZIKV sequences are listed in FIG. 40.

ZIKV DVGs sequences are selected from the group consisting of: SEQ ID NO: 22 (ZIKV DVG-A), SEQ ID NO: 23 (ZIKV DVG-B), SEQ ID NO: 24 (ZIKV DVG-C), SEQ ID NO: 25 (ZIKV DVG-D), SEQ ID NO: 26 (ZIKV DVG-E), SEQ ID NO: 27 (ZIKV DVG-F), SEQ ID NO: 28 (ZIKV DVG-G), SEQ ID NO: 29 (ZIKV DVG-H), SEQ ID NO: 30 (ZIKV DVG-I), SEQ ID NO: 31 (ZIKV DVG-J), SEQ ID NO: 32 (ZIKV DVG-K) and SEQ ID NO: 33 (ZIKV DVG-L).

Preferred ZIKV DVGs sequences are selected from the group consisting of: SEQ ID NO: 22 (ZIKV DVG-A), SEQ ID NO: 24 (ZIKV DVG-C) and SEQ ID NO: 25 (ZIKV DVG-D).

Most preferred ZIKV DVGs sequences are slected from the group consisting of: ), SEQ ID NO: 26 (ZIKV DVG-E), SEQ ID NO: 32 (ZIKV DVG-K), SEQ ID NO: 33 (ZIKV DVG-L) and other possible DVGs within a cluster with the following characteristics; deletion results in in-frame deletion events in which the start site is between positions 500 and 900 nt, and in which the stop site of the deletion is between positions 2800 and 3400 nt of the ZIKV genome.

### Example 4: Chikungunya Virus (Chikv)

In this example, we document and characterize naturally occurring DVGs bearing deletions, arising during chikungunya virus infection in vitro and in vivo. From these, we selected the DVGs most likely to have interfering capacity based on their frequency and recurrence between replicates and/or their ability to be carried over multiple passages. We show that natural DVGs have a strong antiviral activity on chikungunya virus both in mammalian and mosquito cells in vitro, and demonstrate that interfering DVGs can be broad-spectrum against other alphaviruses. Finally, we show that DVGs can be introduced into the mosquito vector prior to infection to inhibit arbovirus dissemination.

Vero, Huh7, 293T and BHK cells were grown in Dulbecco’s modified Eagle’s medium (DMEM), containing 10% fetal calf serum (FCS), 1% penicillin/streptomycin (P/S; Thermo Fisher) and 1% non essential amino-acid (Thermo Fisher) in a humidified atmosphere at 37° C. with 5% CO₂. U4.4 and Aag2 cells were maintained in Leibovitz’s L-15 medium with 10% FCS, 1% P/S, 1% non-essential amino acids (Sigma) and 1% tryptose phosphate (Sigma) at 28° C.

The viral stocks were generated from CHIKV infectious clones derived from the Indian Ocean lineage, ECSA genotype (IOL; described in ¹⁷) or from the Caribbean strain, Asian genotype (Carib; described in ¹⁸). To test DVG interference activity, a CHIKV infectious clone containing the Gaussia luciferase gene under a subgenomic promoter was used (obtained from Andres Merits). In vitro transcription (IVT) with SP6 mMESSAGE mMACHINE kit (Invitrogen) was performed on Not I linearised plasmids prior to transfection in BHK using lipofectamine 2000 (Invitrogen). After one passage in Vero cells, the stocks were titered and kept at -80° C. before use.

Viral titration was performed on confluent Vero cells plated in 24-well plates, 24 hours before infection. Ten-fold dilutions were performed in DMEM alone and transferred onto Vero cells. After allowing infection, DMEM with 2% FCS, 1% P/S and 0.8% agarose was added on top of cells. Three days post infection, cells were fixed with 4% formalin (Sigma), and plaques were manually counted after staining with 0.2% crystal violet (Sigma).

Cells were seeded in 24-well plates to reach approximately 80% confluency the next day. For passage 1, virus was diluted in PBS to obtain a multiplicity of infection (MOI) of 5 PFU/cell (high MOI). Cells were incubated with the viral inoculum at 37° C. for 1 hour. Following virus adsorption, the inoculum was removed, the infected cells were washed with PBS, and replaced with the appropriate cell culture medium containing 2% (v/v) FCS. At 48-72 hour post infection, supernatant was harvested and clarified by centrifugation (12 000 x g, 5 min). The following passages were performed blindly, using a high volume (300 µl) of the clarified supernatant from previous passage to infect naive cells followed by the same procedure. A total of 10 passages were performed. Each passage was titered by plaque assay to determine at which passages DVG accumulation may have resulted in interference. At least three replicates were performed per cell type.

Sequencing. RNA of 100 ul of each sample supernatant was extracted using TRIzol reagent (Invitrogen) or ZR viral RNA kit (Zymo) following the manufacturer’s protocol. RNA was eluted in 15-30 µL nuclease-free water. After quantification using Quant-iT RNA assay kit (Thermo Fisher Scientific), RNA libraries were prepared with NEBNext Ultra II RNA Library kit (Illumina). Multiplex oligos (Illumina) were used during library preparation. The quality of the libraries was verified using a High Sensitivity DNA Chip (Agilent) and quantified using the Quant-iT DNA assay kit (Thermo Fisher Scientific). Sequencing of the libraries (diluted to 1 nM) was performed on a NextSeq sequencer (Illumina) with a NextSeq 500 Mid Output kit v2 (Illumina) (151 cycles).

Next generation sequencing data analysis. All analyses were performed using BBTools suite (Bushnell B. - sourceforge.net/projects/bbmap/). First, fastq files generated from sample sequencing were trimmed for low-quality bases and adaptors using BBDuk. Then, alignment was performed using BBmap and the appropriate CHIKV reference sequence (Carib - GenBank accession no. LN898104.1, IOL -GenBank accession no. AM258994). BBMap’s variant caller, CallVariants, reported deletion events, and the overall DVG frequency per sample was calculated as the sum of the number of junction read counts (n) corresponding to each DVG and normalised as 5/3 × n/N, where N denotes the 98th percentile of the coverage per position. Multiplying by 5/3 aims to correct for the detection limit. Indeed, even if reads are 150 nucleotides long, aligned portions are usually only 30 to 120 nucleotides long. This implies that a read starting 30 or less nucleotides upstream of the breakpoint will be aligned on the right side but not on the left side of the breakpoint, and thus, will not be considered as a DVG. Consequently we are missing (30+30)/150 = 60/150 = ⅖ of the deletions, and the counts must be multiplied by 5/3 as a correction factor. Heatmaps illustrate the deletion score per nucleotide position based on deletion events removing that particular position. Specifically, scores were computed as the sum of the number of reads per million reads (RPM) supporting the deletion of a specific nucleotide position. For plotting start/stop breakpoints, deletions with lengths below 10 nucleotides were discarded.

Cloning selected defective genomes. Defective genomes selected from passages were cloned in the CHIKV infectious clones (under SP6 promoter) corresponding to their strain, using the previously described In Vivo Assembly (IVA) method¹⁹ or using In-Fusion reagent (Takara Bio Reagent). Primers were designed using SnapGene software, with a melting temperature of 60° C., and obtained from Integrated DNA Technology (IDT). 50 µl PCR using either Phusion high fidelity DNA polymerase (Thermo Fisher) or Q5 DNA polymerase (NEB) was carried out with a melting temperature of 57° C. for Phusion enzyme or 65° C. for Q5 polymerase. 18 cycles were carried out and the PCR products were then Dpn I(Thermo Fisher)-treated for at least 2 hours to remove the plasmid template and purified (Macherey Nagel PCR and gel purification kit). When IVA technique was used, 2 ul of the PCR products were directly transformed in XL10-Gold extra competent cells (Stratagene) according to supplied protocol. When In-Fusion reaction was needed, PCR products were ligated using In-Fusion reagent (Takara Bio Reagent) following supplier instructions and 1 ul was transformed in Turbo cells (NEB).

Luciferase assay to test DG interference activity. IVTs were performed using the SP6 mMESSAGE mMACHINE kit (Invitrogen) from Not I linearized infectious clones of DVG and CHIKV Carib-GLuc (see above). RNA production was quantified by Qubit RNA TS Assay kit (Thermo Fisher) and diluted to be at the indicated molar ratios compared to the Carib-GLuc CHIKV. Mixed DVG and full genome RNA were transfected in 293T or U4.4 cells seeded in 96-well plates with TransIT-mRNA transfection kit (Mirus) following the supplier’s protocol (25 ng of Carib-GLuc CHIKV per well). The medium was changed 4 hours post transfection to avoid cellular toxicity. 48 hours after transfection, supernatant was collected and mixed with coelenterazine (supplied by Y. Janin, Institut Pasteur) at a final concentration of 0.05 µM and luminescence was measured on a Tecan Infinite 200 microplate reader. When tested with non-luciferase expressing viruses, the procedure was followed the same way but the supernatant was titered by plaque assay 48 hours post transfection.

RT-qPCR to test for DVG self-replication. 50 ng of DVG or CHIKV Carib IVT was transfected in 293T cells (seeded the day before in 48-well plates), in triplicate, as described above. 8 hours post transfection, supernatant was removed, and cells were washed 3 times with PBS before adding fresh medium. At 8, 20, 28 and 44 hours post-transfection, 200 ul of lysis buffer from ZR 96 viral RNA kit (Zymo) was added on the cells after supernatant removal and stored at -20° C. until all time points were collected. Cellular RNA was extracted with the ZR 96 viral RNA kit and eluted in 15 µl. TaqMan RNA-to-Ct One-step RT-PCR kit (Applied Biosystems) was used to perform a quantitative RT-PCR spanning the 5’UTR-nsP1 region, with 5′-GAGACACACGTAGCCTACCA-3′ as the forward primer, 5’-GGTTCCACCTCAAACATGGG-3′ as the reverse, and 5’- [6-FAM] ACGCACGTTGCAGGGCCTTCA-3′ as the probe. After 20 min at 50 °, and 10 min at 95 °, 40 cycles were performed (95° C. for 15 seconds followed by 60° C. for 1 minute).

Mosquitoes. Aedes aegypti female mosquitoes belonging to the 7th generation from wild mosquitoes collected in Kamphaeng Phet Province (Thailand) were grown at 28° C., 70% relative humidity and a 12 hour light: 12 hour dark cycle, and fed with 10% sucrose. A day before blood feeding, 6 to 10-day old females were selected and starved for a day. A blood meal containing 10⁶ PFU/ml of CHIKV was offered to a pool of 60 females through a membrane feeding system (Hemotek Ltd) set at 37° C. with a piece of desalted pig intestine as the membrane. Following the blood meal, fully engorged females were selected and incubated at 28° C., 70% relative humidity and under a 12 hour light: 12 hour dark cycle with permanent access to 10% sucrose. After 7 days, the mosquitoes were sacrificed and dissected to collect heads, thorax, midgut, legs and wings (legs/wings), and abdominal wall (body). Each organ was ground in 300 ul of L15 supplemented with 2% FBS with Qiagen TissuLyser 2 machine, then clarified by centrifugation before titration and RNA extraction with TriZol reagent for RNA deep sequencing. One hundred and fifty nanograms of RNA produced by in vitro transcription (as described above) and purified by phenol-chloroform was mixed with Leibovitz’s L-15 medium and Cellfectin II Reagent (Thermo Fisher) following the supplier’s instructions. For each condition, forty 6 to 8 day-old females were injected intra-thoracically with 300nl of this mix with Nanoject III Nanoliter Injector (Drummond scientific company). Two days later, after a night of starvation, mosquitoes were fed with an infectious blood meal of 10⁶ PFU/ml of CHIKV Carib-GLuc as described above. After 5 days, mosquitoes were sacrificed; midgut and carcass were dissected and ground as mentioned before. Each sample was then tested for luciferase activity and titered by plaque assay.

Generation of defective viral genomes (DVG) by in vitro high MOI passage. In order to capture deleted DVGs that may arise in cell culture, the Caribbean strain of chikungunya virus (CHIKV Carib) was serially passaged in triplicate at high MOI in mammalian (Vero, Huh7) and mosquito (Aag2, U4.4) cells. RNA was extracted from the clarified supernatant of each replicate at each passage and RNA deep sequencing was performed. Reads were analyzed with the Bbmap pipeline to describe and quantify DVGs by identifying sequence reads that contain deletions. As expected, the amount of DVGs between the first and last passages increased by between 1 and 5 orders of magnitude in all cell types, and the rates and oscillations differed with each cell type (FIG. 33A).

Characterization of the DVGs generated in vitro. Next, we pooled the data for all passages and replicates from each cell, to identify potential deletion hotspots in the different cell types. The resulting heat map (FIG. 33B) reveals the frequency at which each nucleotide position was deleted (regardless of the total length of the deletion), where the x-axis indicates the nucleotide position along the full chikungunya virus genome. Each row represents a different cell type. We observed clear differences in regions with higher frequencies of deletion between cell types, suggesting that the same viral strain generates different deletion variants depending on the host environment. In Vero and Huh7 cells, deletions were abundant in a region spanning the non-structural proteins nsP1-nsP2, yet the hotspot profile was shorter in HuH7 compared to Vero cells. In Aag2 cells, the probability of deleting a nucleotide was more widely spread across the genome. Contrary to the other cell lines, where deletions occurred in the first half of the chikungunya virus genome, DVGs generated in U4.4 cells more often exhibited deletions in the second half of the genome (from nucleotides 6000 [nsP3] to 11500 [3’UTR]). Of note, the same experiment with the CHIKV IOL (Indian Ocean Lineage) strain had similar deletion profiles as the CHIKV Carib strain in Vero and Aag2 cells. However, CHIKV IOL deletions differed strongly in Huh7 (where the strongest hotspot is from nucleotides 9000 [E2] to 11000 [3’UTR]) and in U4.4 cells (whose profile in this case was similar to the profile obtained in Aag2 cells) (FIG. 33B). Taken together, these results suggest that both the virus strain, as well as the cellular environment, influence the generation and maintenance of DVGs during passaging in vitro. To visualize the different DVGs in these viral populations, the specific start (x axis) and stop (y axis) breakpoints of individual DVGs with deletions of >100 nucleotides were plotted (FIG. 33C). The analysis revealed predominant DVGs forming three clusters (cluster A, B and C) based on the location and the size of the deletion that was observed in all cell types, while a fourth cluster (cluster D) was more prominent in mosquito cells. Cluster A presented relatively small deletions of approximately 2500 nt, between the nsP1 and nsP2 genes. Cluster C presented large deletions of over 9000 nt from the end of nsP2 to the beginning of the 3’UTR, and for cluster B, an even larger deletion spanning nsP1 to the 3’UTR. In Aedes mosquito cells (Aag2 and U4.4) but less so in mammalian cells, another cluster D was present with deletions of approximately 5000nt between nsP3 to the 3’UTR.

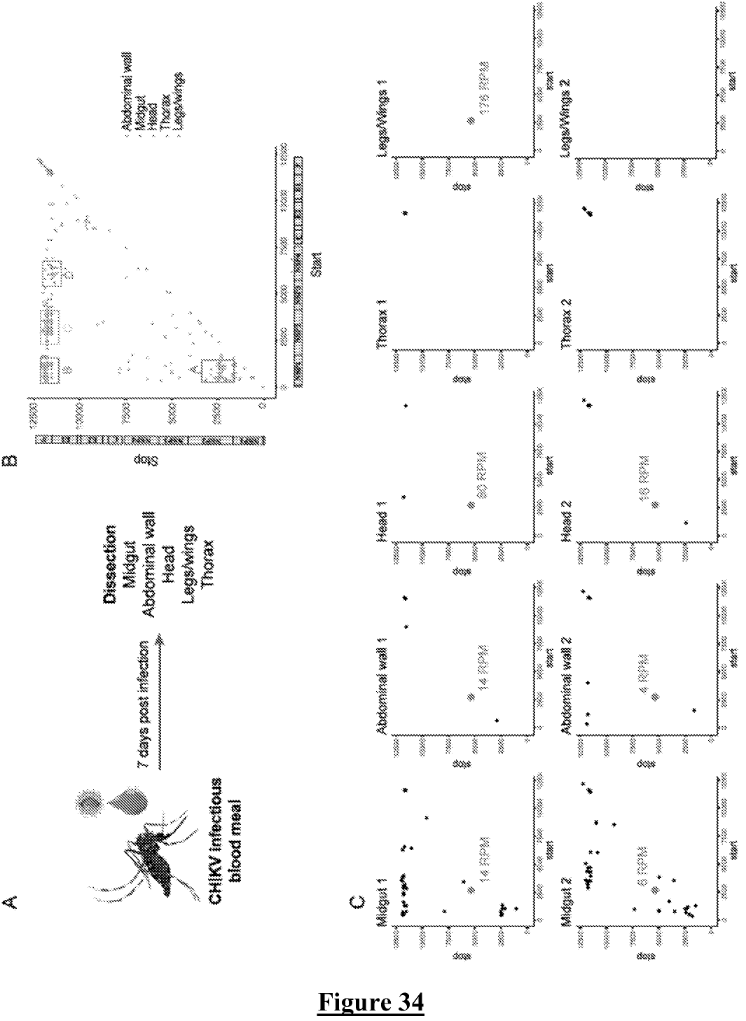

Generation of chikungunya DVGs in mosquitoes in vivo. To see if DVGs were also generated during in vivo mosquito infection, we fed Aedes aegypti mosquitoes an infected blood meal containing 10⁶ PFU/ml of the Caribbean strain of chikungunya virus. After allowing the infection to disseminate throughout the mosquitoes over seven days, we sacrificed and dissected ten of them to collect individual organs: midgut, abdominal wall (body), thorax, heads and legs/wings (FIG. 34A). RNA was extracted and sequenced, and the data were analyzed using the BBmap pipeline. The plots of the DVG start and stop positions showed similar patterns to the data generated in Aag2 cells (FIG. 34C), with four distinct clusters (FIG. 34B). In two mosquitoes out of ten (FIG. 34C), we found the same DVG (named CM1) in several organs (midgut, body, head and legs/wings for one mosquito and midgut, body and head for the other mosquito). No other mosquitoes, nor the viral stock used to infect them, contained this DVG-CM1. These results indicate that DVGs are readily generated in the mosquito host, and that they share similar deletion profiles as what was observed in cell culture.

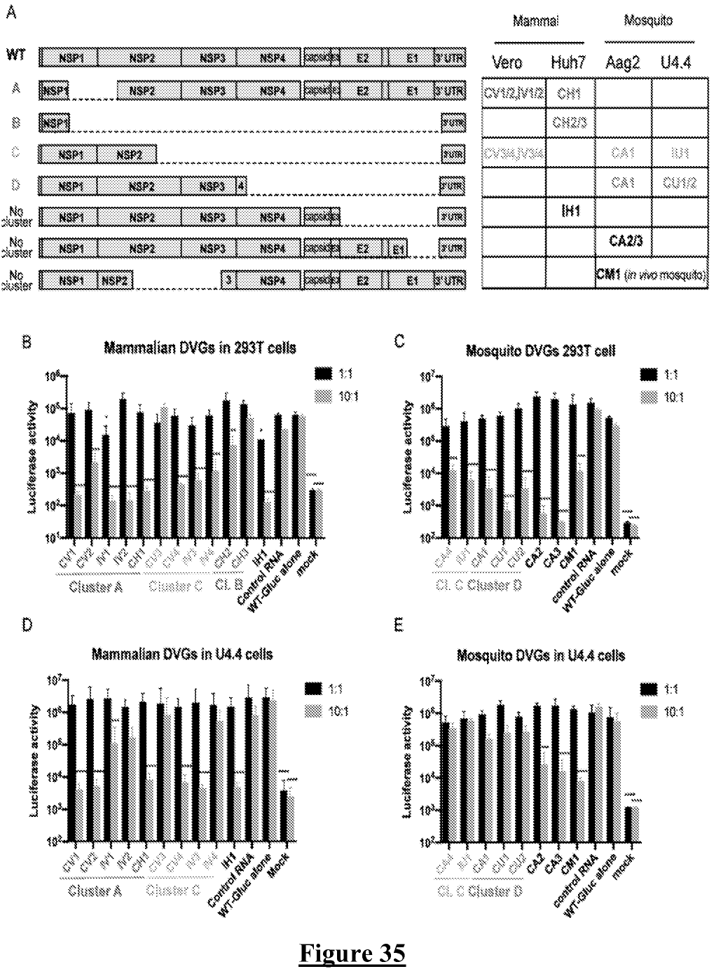

Chikungunya DVG candidates are non replicative. Our goal in the previous sections was to uncover all of the possible DVGs generated during virus infection and to down-select from the hundreds of individual DVGs, those that would most likely and efficiently compete with wild type virus. In other words, to identify which of the total DVGs would be the most potent defective interfering particles. Our rationale was that such DVGs would occur more frequently at high MOIpassage, and would increase to higher frequency over time as the DVG competes with wildtype virus for resources. DVGs were thus selected based on criteria that would be indicative of higher fitness vis à vis the wildtype competitor: having high frequency, being maintained throughout the passage series and occurring in several replicates. In addition to DVG-CM1, carrying a deletion from nucleotide 2695 (middle of nsP2) to nucleotide 5358 (end of nsP3) (FIG. 35A), we selected and cloned another 19 DVGs, from the cell passage deep sequencing data of CHIKV Carib (described above) and CHIKV IOL. Representatives from all four clusters were selected (FIG. 35A). We named DVGs according to their virus strain of origin (C for CHIKV Carib and I for CHIKV IOL) and the cell in which they were generated (V for Vero, H for Huh7, A for Aag2 and U for U4.4, M for in vivo mosquito). Their genomic composition is shown in FIG. 35A and their exact deletions are listed in Table 1. Three additional candidates did not belong to any defined cluster, but were present at high frequency in several replicates and were maintained throughout passages (Table 1). To confirm that the selected DVGs were indeed defective genomes, unable to self-replicate in absence of virus, we transfected in vitro transcribed RNA of each DVG or wild type virus in 293T cells. We harvested each DVG- or control-transfected cells at 8, 20, 28, and 44 hours post transfection, extracted RNA from the cells and performed a RT-qPCR. Contrary to CHIKV Carib full-length virus RNA that had an increasing RNA copy number throughout the timepoints, all DVG RNA copy numbers decreased over time, reflecting the progressive degradation of transfected RNA, and demonstrating that none of the candidate DVGs could self-replicate (FIG. 38).

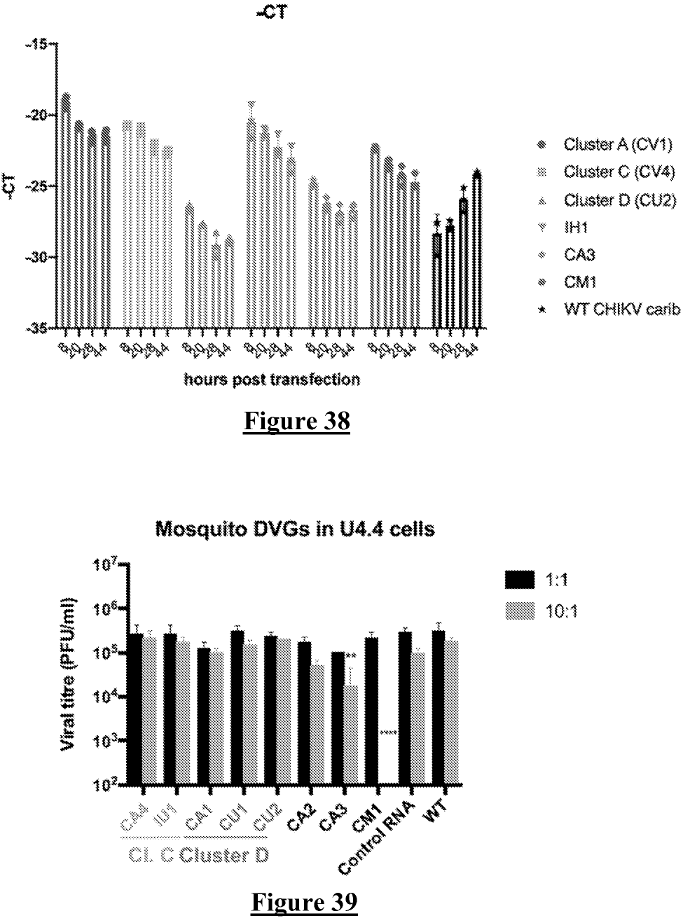

Chikungunya DVGs interfere with wild type virus in vitro. We next tested the interference activity of the 20 DVGs derived from either mammalian cell culture (FIG. 39B) or mosquito host environments (FIG. 39C) in mammalian cells. We chose 293T cells for their high transfectability, and a CHIKV Carib virus expressing Gaussia luciferase under a sub-genomic promoter (CHIKV Carib-Gluc) as the target virus for rapid quantification. To test the DVGs that were derived from mammalian or mosquito cell culture, 293T cells were transfected with a mix of in vitro transcribed RNA corresponding to one DVG of interest (or a control RNA) and the CHIKV Carib-Gluc full length virus RNA at a 1:1 or 10:1 molar ratio. Supernatants were harvested at 48 hours to measure luciferase activity as a surrogate measure for wild type virus replication, since titers and luminescence correlated (FIG. 39). At a 1:1 molar ratio, most DVGs did not reduce wild type virus luciferase expression, but transfection of IV1 and IH1 showed a modest decrease (p< 0.05). However, increasing the amount of DVG RNA to 10 times that of CHIKV Carib-Gluc RNA significantly inhibited virus replication in nearly every case by 1 to 3 orders of magnitude (FIGS. 34B and C). Of note, the smallest DVGs with the largest deletions (CH2 and CH3 from cluster B) had no, or little, interference activity at the highest DVG:CHIKV Carib-Gluc molar ratio, with a decrease of less than 1 log (FIG. 35B). These results show that DVGs derived from both mammalian or mosquito cell culture can inhibit virus replication in mammalian cells when introduced exogenously.

To determine whether these mammalian and mosquito cell-derived DVGs could also inhibit virus replication in mosquito cells, we repeated the experiment in Aedes albopictus cells (U4.4). As for 293T cells, no interference was observed at a 1:1 molar ratio; but considerable inhibition with decreases of 1 to 2 log of luciferase activity was observed at 10:1 ratios for all of the DVGs that previously inhibited virus in mammalian cells, except for IV4. As seen in 293T cells, no interference was observed for DVG CV3 in mosquito cells (p>0.05) (FIG. 35D), which along with CH3, were the only two of 20 DVG candidates to fail to inhibit virus in any condition. On the other hand, most of the mosquito cell-derived DVGs that could inhibit virus in 293T cells completely lost their interference activity in mosquito cells; only DVG CA2, CA3 and CM1 significantly reduced luciferase activity by 1 to 2 log in U4.4 cells at a molar ratio of 10:1 (FIG. 35E).

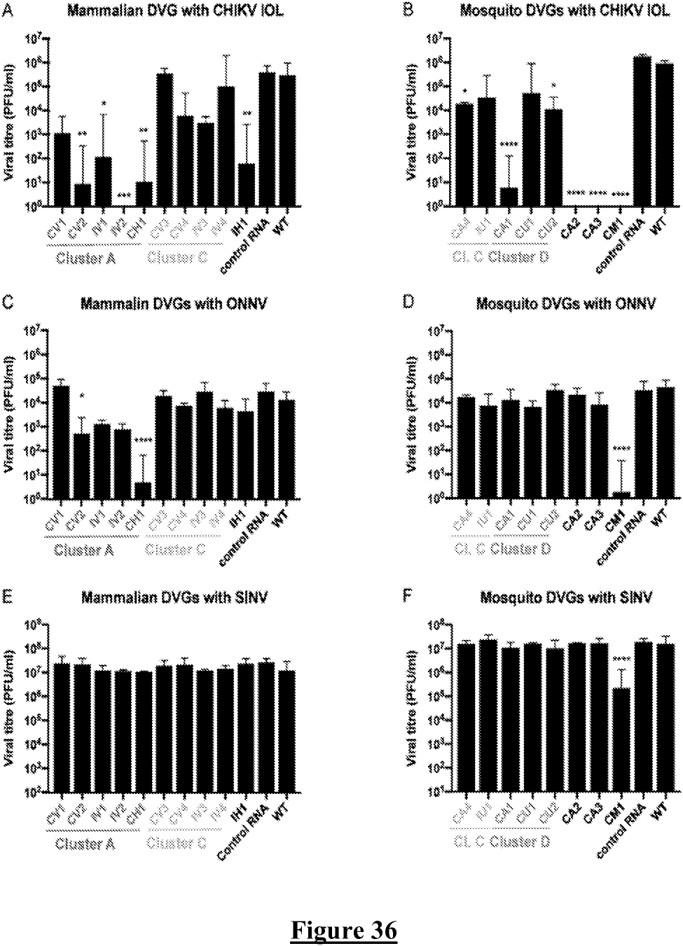

Chikungunya DVGs can be broad-spectrum inhibitors. The chikungunya virus Indian Ocean lineage belongs to the East, South and Central African (ECSA) genotype and has approximately 7% nucleotide divergence with the chikungunya virus Caribbean strain that belongs to the Asian genotype²⁰. In the previous experiments, DVGs derived from either the Indian Ocean Lineage or Caribbean strains were tested against the Caribbean strain virus expressing Gaussia luciferase. Importantly, all of the CHIKV IOL-derived DVGs inhibited the Caribbean strain, showing that DVGs can act broadly within the same virus species (FIGS. 35B,C,D,E). To extend these results, we repeated the same experiment in 293T using CHIKV IOL strain as the target virus. In this case, we measured inhibition by virus titer instead of luminescence. Most DVGs resulted in reduced virus titers, either modestly or significantly, whether they were identified from mammalian (FIG. 36A) or mosquito (FIG. 36B) environments.

We next investigated whether CHIKV DVGs could inhibit even more broadly within the alphavirus genus. We carried out the same experiment in 293T cells with O’nyong’nyong virus (ONNV, CHIKV’s closest relative) or Sindbis virus (SINV, a very distant relative) as the targets. While the inhibitory effect was lost for most DVGs, some DVGs still significantly inhibited O’nyong’nyong virus (CV2 and CH1) (FIG. 36C). Of the mosquito-derived DVGs, CM1 maintained inhibitory activity with close to no detectable O’nyong’nyong virus titers (FIG. 36D). When tested against the distantly related Sindbis virus, interference activity was lost for all DVGs, but for CM1 that inhibited Sindbis virus by 2 logs (FIGS. 36E,F). These results confirm that some DVGs derived from one alphavirus can have inhibitory activity on different strains, including closely, and in some cases more distantly related viruses from the same genus.

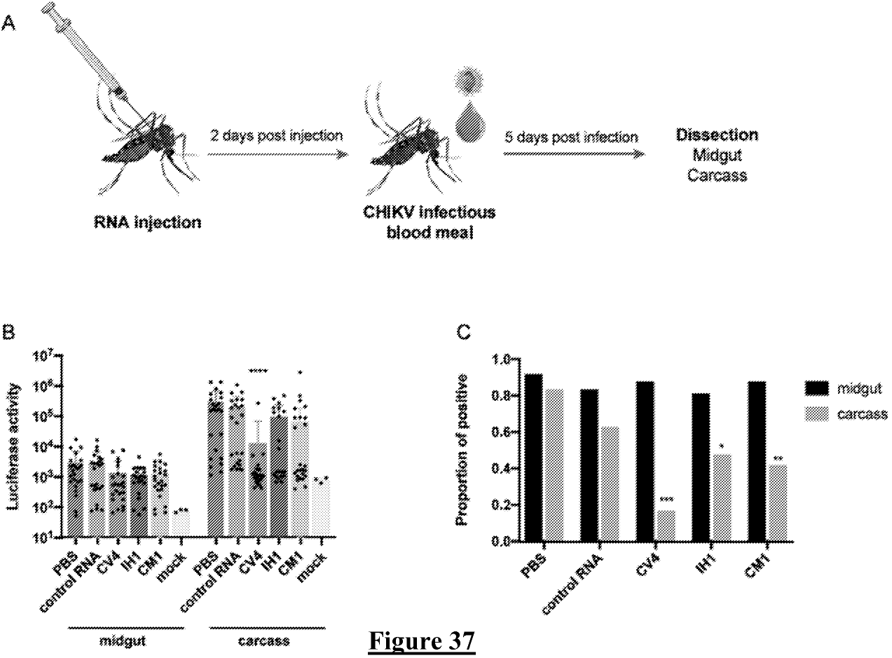

Chikungunya DVGs block viral dissemination in vivo, in Aedes aegypti mosquitoes. After demonstrating that DVGs can be used to limit/inhibit infection in vitro, we tested if their interference activity could be used to block infection or dissemination in the mosquito host. To do so, we injected purified RNA of the CV4, IH1 or CM1 DVGs, a control RNA, or PBS into Aedes aegypti mosquitoes 2 days prior to feeding them with a blood meal containing the CHIKV Carib-Gluc virus. Five days post infection, mosquitoes were sacrificed and midguts were dissected from the rest of the carcass (FIG. 37A). Virus replication was measured in each mosquito by quantifying luciferase activity. Replication in the midgut was similar in all mosquitoes regardless of whether interfering DVG candidates were present or not. However, replication was significantly reduced in CV4- and CM1-treated mosquito carcasses (FIG. 37B). Virus in midguts (representing infection) and carcass (a proxy for viral dissemination) was then quantified, and classified as positive or negative, depending on whether infectious viruses were detected or not (limit of detection 30 PFU per organ). All groups had a similar proportion of positive midguts (between 81 and 91.7%) (FIG. 37C), confirming that the number of infected mosquitoes following bloodmeal feeding was similar for each group. On the other hand, virus dissemination was significantly impacted by the presence of DVGs: mosquitoes injected with either CV4, IH1 or CM1 DVGs had lower dissemination rates compared to PBS injected mosquitoes (16.7%, 47.6% and 41.7% of total infected mosquitoes in each group) (FIG. 37C). These results confirm that DVGs can impact viral replication and dissemination in vivo, in the mosquito host

FIG. 33: Generation of chikungunya virus (CHIKV) defective viral genomes (DVGs) in different hosts in vitro. The Caribbean strain (Carib) and Indian Ocean lineage (IOL) of CHIKV was passaged at high MOI in mosquito (U4.4, Aag2) or mammalian (Vero, Huh7) cells in triplicate. For each passage, the supernatant harvested from the previous passage was used to infect fresh cells. The infection lasted 48 to 72 hours. (A) DVG accumulation through passages. Total frequency of CHIKV Carib DVG arising in each replicate in 2 mammalian cell lines (Vero, Huh7) and 2 mosquito cell lines (Aag2, U4.4), determined after RNA deep-sequencing of each sample and quantification of DVG with Bbmap pipeline output. Samples with coverage under 200 were excluded from analysis. DVG count is represented in log scale (y axis) at each passage (x axis). (B) CHIKV Carib or IOL DVG heat maps in different cell types. The viral population of each passage was deep sequenced (RNAseq) and analyzed through BBmap pipeline. An average of all passages and replicates of the normalized frequency of each deleted nucleotide position (x axis) throughout the full genome is shown as a heat map (shades of orange), for each different cell type (y axis). (C) Analysis of the deletions generated by high MOI passages by CHIKV Carib. Start (x axis) and stop (y axis) positions of the breakpoint of DVGs generated are plotted for each cell types. The different clusters are called A, B, C, D.

FIG. 34: Generation of chikungunya DVGs in mosquitoes in vivo. (A) Thai Aedes aegypti mosquitoes were fed a blood meal infected with 10⁶ PFU/ml of CHIKV Carib. After 7 days, ten mosquitoes were sacrificed and midgut, abdominal wall (body), thorax, head and legs/wings were dissected. After verification of infection by titration, viral RNA was extracted and deep-sequenced. (B) Analysis of the deletions generated by CHIKV Carib in nine infected mosquitoes pooled together. Start (x axis) and stop (y axis) positions of the breakpoint of DVGs generated are plotted for each organs. (C) Start (x axis) and stop (y axis) positions of the breakpoint of DVGs generated in the different organs of 2 individual mosquitoes. The red dots represent the deletion starting at 2695 and ending at 5358, this DVG is called CM1. The number next to it indicates the number of reads per million reads (RPM).

FIG. 35: Defective viral genomes can interfere with chikungunya virus replication. (A) Schematic of the DVG candidates and the cell type and strain from which they were (C Carib, I IOL). (B-E) Measurement of DVG activity in vitro. Each DVG candidate identified from mammalian cell passage (B,D) or mosquito cell passage (C,E) was co-transfected with CHIKV Carib-luciferase at 1:1 or 10:1 molar ratio in 293T cells (B,C) or U4.4 cells (D,E). After 48 hours, luciferase activity was measured. (Representative result of one experiment out of three independent experiments is shown, n=3, ^{∗}p<0.05, ^{∗∗}p<0.01, ^{∗∗∗}p<0.001, ^{∗∗∗∗}p<0.0001, ns non significant compared to wild type after Dunnett’s multiple comparisons on one-way ANOVA test.)

FIG. 36: Defective viral genomes have broad-spectrum inhibiting activity in the alphavirus family. The DVGs identified in mammalian cell passage (A,C,E) or mosquito cell passage (B,D,F) were tested for inhibition of CHIKV IOL (A, B), O’nyong-nyong virus (ONNV) (C, D) and Sindbis virus (SINV) (E, F). DVG candidates were co-transfected with CHIKV IOL (A,B), ONNV (C,D) and SINV (E,F) at 10:1 molar ratio in 293T cells. After 48 hours, viral titer was measured. (Representative result of one experiment out of three independent experiment, n=3, ^{∗}p<0.05, ^{∗∗}p<0.01, ^{∗∗∗}p<0.001, ^{∗∗∗∗}p<0.0001, ns non significant compared to wild type after Dunnett’s multiple comparisons on one-way ANOVA test)

FIG. 37: Defective viral genomes prevent viral dissemination of chikungunya virus in Aedes aegypti mosquitoes. (A) 150 ng of DVG candidate RNA (IH1, CV4 or CM1), a control RNA or PBS were injected into Aedes aegypti mosquitoes 2 days prior to being fed an infected blood meal containing 10⁶ PFU/ml. After 5 days, mosquitoes were sacrificed, and midguts were separated from carcasses. (B) Luciferase activity of midgut or carcass of each mosquito was measured. (C) After virus titration, the proportions of positive midguts or carcasses (eg PFU≥100 PFU/ml) were determined. (Representative result of one experiment out of two independent experiments is shown, n=24, ^{∗}p<0.05, ^{∗∗}p<0.01, ^{∗∗∗}p<0.001, ns non significant compared to PBS after one-way ANOVA with multiple comparison (B) or Chi square test, multiple comparison with Holm correction (C)).

FIG. 38: Chikungunya virus DVGs are not self-replicating. 293T cells were transfected with DVG or CHIKV Carib in vitro transcribed RNA and harvested at 8, 20, 28 and 44 hours post transfection. Cellular RNA was extracted and used to perform a RT-qPCR with a Taqman probe

FIG. 39: Measuring DVG activity in vitro by virus titration. In vitro transcribed RNA of each DVG candidate (derived from mosquito cells) was co-transfected with CHIKV Carib-luciferase RNA at 1:1 or 10:1 molar ratio in U4.4 cells. After 48 hours, samples were titered and luciferase activity was measured (FIG. 34E). (n=3, ^{∗∗}p<0.01, ^{∗∗∗∗}p<0.0001, ns non significant compared to wild type after Dunnett’s multiple comparisons on one-way ANOVA test.).

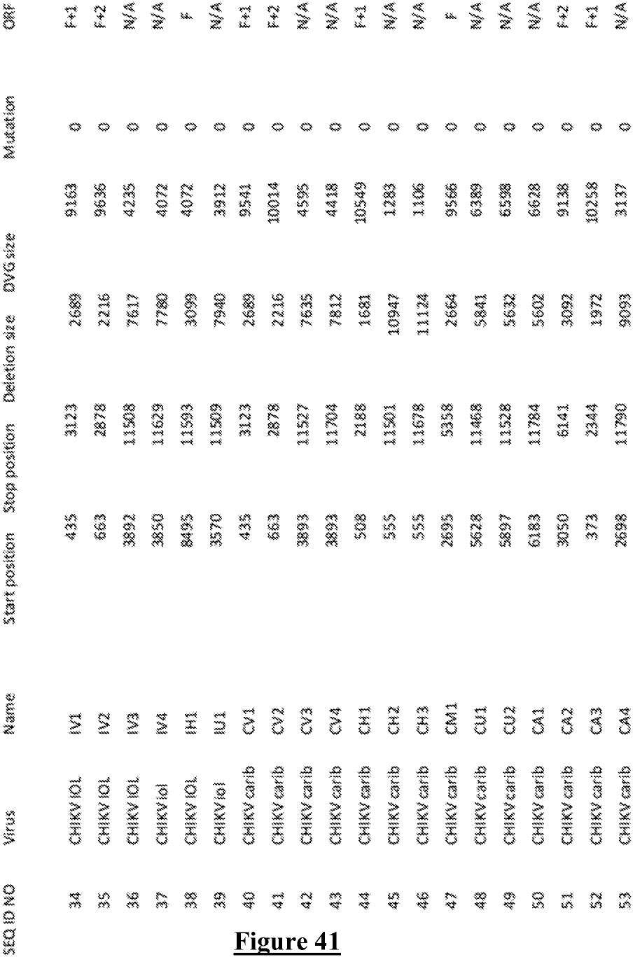

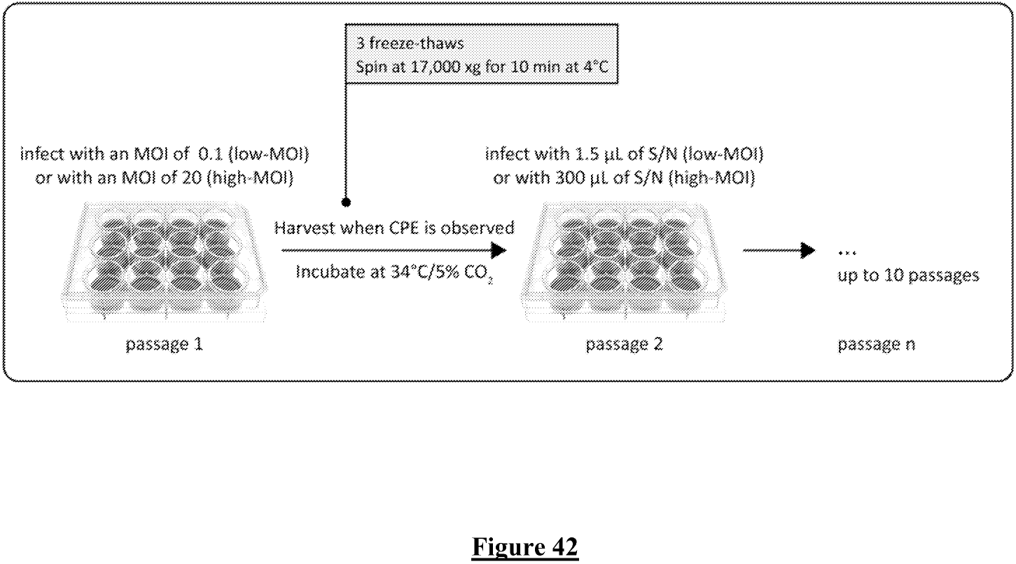

FIG. 41 lists characteristics of CHIKV DVGs studied. DVG, defective viral genomes; CHIKV, chikungunya virus; Deletion location, the region of the genome affected by the deletion that may include envelope proteins (E), nonstructural proteins (NSP) or 3’untranslated region (3’UTR).

### Discussion

Described in nearly all RNA virus families^{6,21} and often considered a waste product of viral replication, defective interfering particles, and more broadly speaking DVGs, have recently garnered attention for their possible use as antiviral tools^{15,6,22-26}. Indeed, the presence of DVGs in natural human infections correlates to milder disease in clinical studies on respiratory syncytial virus and influenza virus^{10,13}; and influenza defective interfering particles protect mice against lethal challenge^{22,23,27}. In this study, we aimed to characterize, as widely as possible, the DVGs bearing deletions that arise during chikungunya virus infection in vitro in mammalian and mosquito environments and in vivo in its mosquito host. We show that chikungunya DVGs arise in all environments, in vitro and in vivo, but their type and abundance depend on the host and cell type, and on the virus strain, highlighting that both cellular environment and the viral genome bear determinants of DVG generation. For example, cluster D is strongly represented in both Aedes spp. mosquito cells, even though the deletion does not occur at the exact same position, but it is very rare in mammalian cells.

To find DVGs that could be useful as antivirals, we selected the most redundant DVGs, that arose in several conditions and were propagated over passages, which we assumed to be indicative of higher fitness and the ability to be packaged by wild-type virus. When used in higher quantity relative to wild-type virus, most candidates showed promising inhibiting capacity. Of note, because smaller genomes should be replicated more quickly^{7,8}, the conventional dogma proposes that the smallest DVGs would most efficiently hijack the parental virus replication machinery and outcompete wild-type virus. However, in our work, the smallest DVGs identified (two DVGs from cluster B) had no or very little interference activity. This observation implies that strict competition for the replicase and replication speed is not the only mechanism by which DVGs interfere with parental virus, and that their previously described immunostimulatory activity^{7,9-11,28-33} or the competition for structural protein resources^{8,34} may also play an important role. Importantly, we used Illumina deep sequencing technology that only generates small reads to identify DVGs, making it close to impossible to identify DVGs with several deletions and/or major rearrangements. In the future, combining this approach with a long-read sequencing technology such as nanopore might generate an even more complete picture of the nature of DVGs generated in the chikungunya virus population and inform in their mode of action.

Of the DVGs generated in Aedes aegypti in vivo, CM1 attracted our attention because it could cross bottlenecks in vivo. During a natural viral infection, after an infectious blood meal, the midgut is the first organ to be infected before the virus reaches the hemocoel to disseminate to all other organs³⁵. Several works in mosquitoes have shown that virus exit from the midgut is the main population bottleneck during mosquito infection with a drastic reduction in population size, followed by egress from the salivary glands, a mandatory step for viral transmission to the mammalian host through infected saliva³⁵⁻³⁹. CM1 was newly generated in the midguts of two independent mosquitoes and found to disseminate to the body wall, head and legs/wings of these same mosquitoes. The possibility that CM1 was generated de novo in other organs is unlikely because this DVG never appeared in any organs of any other mosquitoes that had not generated it in the midgut. It is not clear how or why CM1 is able to cross the midgut bottleneck. One possibility is that it belongs to a collective infectious viral unit, a structure that simultaneously contains and transports multiple viral genomes to a single cell, such as polyploid virions, aggregates of virions or virion-containing lipids vesicles^{40,41}. The possibility of collective infectious unit containing DVGs has already been proposed. Indeed, Leeks et al. modeled that if the viral population contains many DVGs, the collective infectious unit could become very large. Nonetheless, the presence of interfering DVGs would disfavor transmission within the collective infectious unit compared to free virions⁴². While CM1 had an inhibiting activity at high molar ratio compared to the parental virus, its frequency in mosquito samples was very low (4 to 176 reads per million).

A valuable characteristic of some of these interfering DVGs is their broad-spectrum activity, with inhibition not only on other chikungunya virus strains but also on other alphaviruses. This cross reactivity had already been reported with influenza and Sendai virus, since their DVGs were shown to be efficient vaccines or vaccine adjuvants in mammalian models not only against the virus from they were derived, but also against unrelated viruses^{15,22-26}. From a therapeutic point of view, this is of particular interest since the risk of worldwide dissemination of arthritogenic alphaviruses is well accepted^{3,43-47}, especially since no antivirals or vaccines against any of these viruses are currently licensed. In this context, any treatment (antiviral or vaccine) that could work across a viral genus or family would be helpful in facing outbreaks to come.

It will be important in the future to test if chikungunya DVGs could function as antivirals in mammalian in vivo models as well; however, a proper delivery system still needs to be developed. In previous work on influenza A and Sendai DVGs, authors isolated DVGs by ultra-centrifugation of a mix of wildtype virus and DVG on sucrose gradients^{15,11-16}. However, we could not successfully separate chikungunya DVGs from wild type virus using these methods. New approaches, such as delivering RNA or DNA in nanoparticles or nanostructured lipids^{48,49}, or attempting to package DVGs in virus-like particles (VLPs) could be explored. Another major hurdle to overcome is the choice of in vivo model. Wild-type C57BL/6 could be used, but as mice develop only a transient, mild infection, it would be difficult to demonstrate an inhibitory effect for even the best DVG candidates. While the most commonly used mouse models rely on interferon α/β knockouts^{50,51}, the induction of innate immunity would be excluded, which may be an important mechanism of action in chikungunya DVGs^{10,28-31,33}.

Finally, when injected in Aedes aegypti mosquitoes two days before an infectious blood meal, three DVGs significantly reduced viral dissemination from the mosquito midgut, thereby shifting the status of mosquitoes from being competent to incompetent vectors for chikungunya virus spread. This result is a proof of principle that DVGs might be useful tools in controlling chikungunya virus infection in the vector population. As releasing incompetent mosquitoes in the wild during an arbovirus outbreak has already been shown to be an effective epidemic control strategy^{52,53}, and despite the need for a simpler delivery system, it is tempting to propose that interfering DVGs could be used as a control strategy not only for chikungunya virus, but for any arbovirus. Furthermore, interfering DVGs are presumably safe antiviral tools because they are inert molecules that cannot self-replicate, and are only active when wild-type virus is present.

In conclusion, this work describes the different types of deleted DVGs generated during chikungunya virus infection in both vertebrate and invertebrate environments in vitro and in vivo in the mosquito vector. Moreover, we identified criteria to down-select the best defective interfering particle candidates able to inhibit wild-type chikungunya virus in vitro in both vertebrate and invertebrate hosts. An interesting observation is the broad-spectrum activity of some interfering DVGs able to interact with related alphaviruses. Finally, we show that pre-exposure to a DVG can modulate viral dissemination in mosquitoes in vivo. These results strengthen the idea that defective interfering particles might be a useful therapeutic tool for chikungunya virus infection as well as an efficient vector control strategy.

CHIKV DVGs sequences are selected from the group consisting of: SEQ ID NO: 34 (CHIKV DVG-IV1), SEQ ID NO: 35 (CHIKV DVG-IV2), SEQ ID NO: 36 (CHIKV DVG-IV3), SEQ ID NO: 37 (CHIKV DVG-IV4), SEQ ID NO: 38 (CHIKV DVG-IH1), SEQ ID NO: 39 (CHIKV DVG-IU1), SEQ ID NO: 40 (CHIKV DVG-CV1), SEQ ID NO: 41 (CHIKV DVG-CV2), SEQ ID NO: 42 (CHIKV DVG-CV3), SEQ ID NO: 43 (CHIKV DVG-CV4), SEQ ID NO: 44 (CHIKV DVG-CH1), SEQ ID NO: 45 (CHIKV DVG-CH2), SEQ ID NO: 46 (CHIKV DVG-CH3), SEQ ID NO: 47 (CHIKV DVG-CM1), SEQ ID NO: 48 (CHIKV DVG-CU1), SEQ ID NO: 49 (CHIKV DVG-CU2), SEQ ID NO: 50 (CHIKV DVG-CA1), SEQ ID NO: 51 (CHIKV DVG-CA2), SEQ ID NO: 52 (CHIKV DVG-CA3) and SEQ ID NO: 53 (CHIKV DVG-CA4).

Preferred CHIKV DVGs sequences are selected from the group consisting of: SEQ ID NO: 34 (CHIKV DVG-IV1), SEQ ID NO: 35 (CHIKV DVG-IV2), SEQ ID NO: 36 (CHIKV DVG-IV3), SEQ ID NO: 37 (CHIKV DVG-IV4), SEQ ID NO: 38 (CHIKV DVG-IH1), SEQ ID NO: 39 (CHIKV DVG-IU1), SEQ ID NO: 40 (CHIKV DVG-CV1), SEQ ID NO: 41 (CHIKV DVG-CV2), SEQ ID NO: 42 (CHIKV DVG-CV3), SEQ ID NO: 43 (CHIKV DVG-CV4), SEQ ID NO: 44 (CHIKV DVG-CH1), SEQ ID NO: 47 (CHIKV DVG-CM1), SEQ ID NO: 48 (CHIKV DVG-CU1), SEQ ID NO: 49 (CHIKV DVG-CU2), SEQ ID NO: 50 (CHIKV DVG-CA1), SEQ ID NO: 51 (CHIKV DVG-CA2), SEQ ID NO: 52 (CHIKV DVG-CA3) and SEQ ID NO: 53 (CHIKV DVG-CA4).

Most preferred CHIKV DVGs sequences are selected from the group consisting of: SEQ ID NO: 41 (CHIKV DVG-CV2), SEQ ID NO: 44 (CHIKV DVG-CH1), SEQ ID NO: 47 (CHIKVDVG-CM1), SEQ ID NO: 38 (CHIKVDVG-IH1) and SEQ ID NO: 43 (CHIKV DVG-CV4).

### Example 5: Rhinoviruses (RV)

### Generation and Characterization of DVGs Occurring Within Rhinoviruses

Method: Rhinovirus (RV) types used for the identification of defective viral genomes (DVGs) include RV-A01a (NC_038311.1), RV-A16 (L24917.1), RV-B14 (L05355.1), and RV-C15 (GU219984.1). Briefly, 12-well plates were seeded with 3.5 x 105 (350,000) H1—HeLa cells/well in complete media (DMEM, 10% FBS, 1% penicillin/streptomycin) and incubated overnight at 37° C./5% CO₂ to reach ~90% confluency. In the case of RV—C15 virus, the HeLa-E8 cell line was used. Next day, the cells were infected with wild type RV-A01a, RV-A16, RV-B14, and RV-C15 viruses at high (20) and low (0.1) multiplicity of infection (MOI). Following an hour of incubation at 34° C./5% CO₂, the virus was removed and the cells were washed with phosphate buffered saline (PBS) solution. Then, PBS was aspirated and one mL of infection media (DMEM, 2% FBS, 1% penicillin/streptomycin) was added. Cells were incubated at 34° C./5% CO₂, until complete cytopathic effect (CPE) was observed. Upon CPE, viruses were harvested by three repeated freeze-thaw cycles, and cell debris was removed by centrifugation (17,000 x g for 10 min at 4° C.). The viral supernatant was aliquoted and stored at -80° C. until ready to use. The supernatant was used to passage the virus into fresh cells. This process was repeated until passage 10 (FIG. 42). Prior to viral RNA isolation, 100 µL of the viral supernatant was treated with 5 µL of RNase A/T1 (Thermo Scientific) and the mixture was incubated for 1 hr at 37° C. After the RNase treatment, 300 µL of TRI reagent (Sigma) was added per 100 µL viral supernatant and mixed to homogeneity. Then, the RNA was isolated using the Direct-zol-96 RNA kit (Zymo Research). In-column DNase treatment was performed by following the manufacturer’s protocol. Five microliters of the isolated RNA (generally ≤1 ng/µL) was used for the library preparation. Libraries were prepared by using the NEBNext Ultra II RNA Library Prep kit for Illumina (New England Biolabs). Multiplex oligos (Illumina) were used during the library process. Quality of the library preparation was checked on a Bioanalyser 2100 (Agilent) using High Sensitivity DNA Chips. Sequencing of the libraries diluted to 1 nM was performed on a NextSeq 500 sequencer (Illumina) with a NextSeq 500 Mid Output kit v2.5 (Illumina) (151 cycles). The obtained sequences were analyzed through an in-house pipeline to identify DVGs.

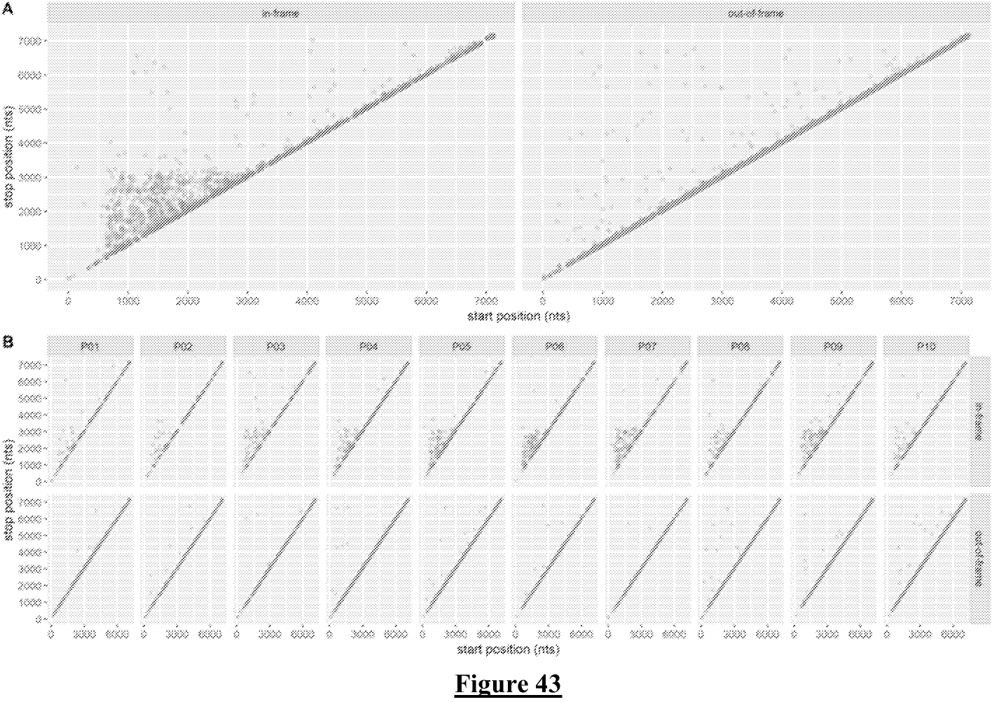

Considering the presence of numerous RV types, we conducted our experiments with RV-A01a, -A16, -B14, and -C15, to make sure that all the RV species are represented within our dataset. These viruses were passaged up to 10 times at low and high-MOI in H1—HeLa cell line as described above (FIG. 42). Deep sequencing of samples and bioinformatics analysis identified all the deletion hotspots across the RV genome within the population. For all the RV types tested, the major deletion hotspots are contained within the P1 region (structural proteins) of the genome and they are all in-frame (FIGS. 43-46). Additionally, all the samples were RNase treated prior to RNA isolation and deep sequencing, suggesting that the identified DVGs can be packaged into the viral capsid. As it relates to the DVGs within the major hotspots, in all cases the cis-acting replication element (cre) was preserved, putting them in replicative advantage compared to the other DVGs.

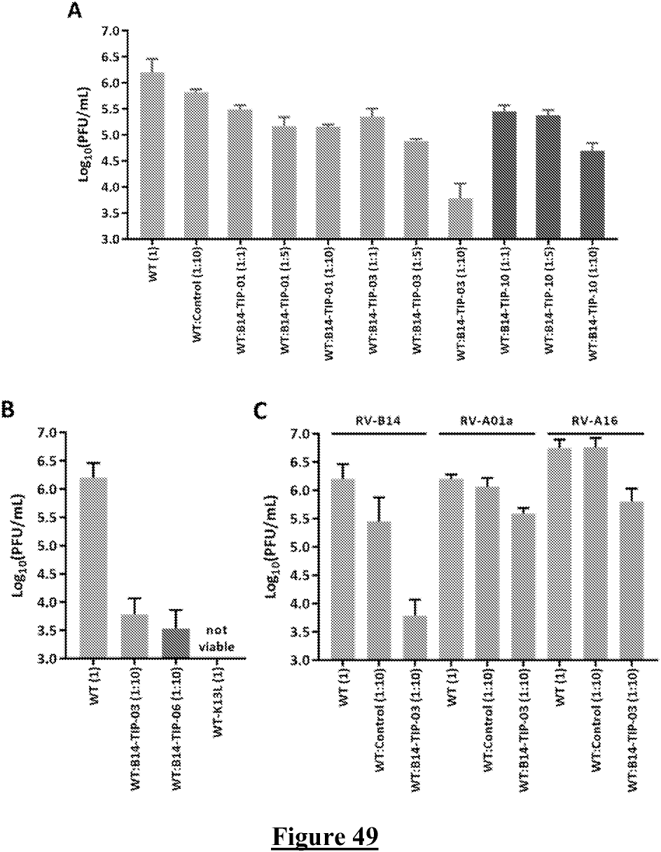

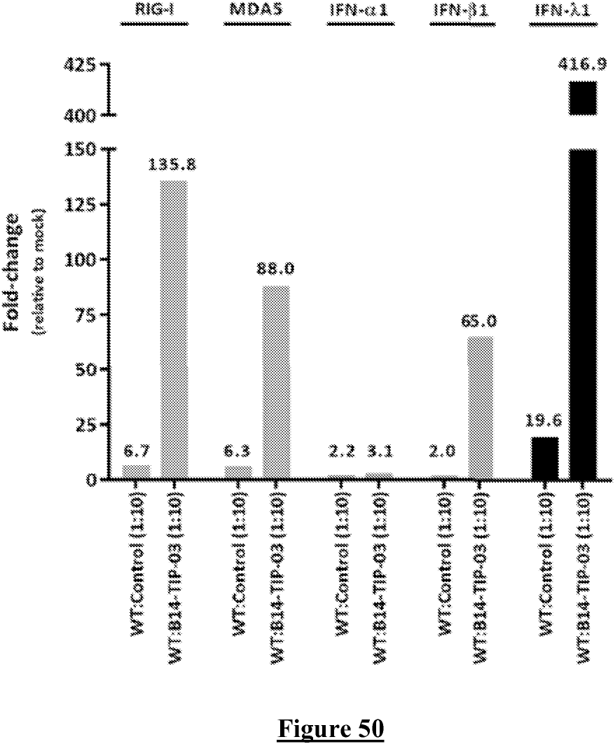

The selection of TIP candidates was done based on the presence of hotspots, location along the genome, size of the deletions, and the frequency of appearance for each deletion within/across passages. The TIP candidates’ names include the RV type followed by the TIP number (e.g. B14-TIP-01) (FIG. 47 and FIG. 48).

FIG. 42. Methodology used for generating rhinovirus defective viral genomes. The overall process included blind passaging at low-/high-MOI, followed by viral RNA purification, deep sequencing, and data analysis to identify defective viral genomes (DVGs). Prior to passaging, 12-well plates were seeded with 3.5 x 10⁵ H1-HeLa cells/well in complete media (DMEM, 10% FBS, 1% penicillin/streptomycin) and then incubated for 24 hrs at 37° C./5% CO₂. For RV-A01a, RV-A16, and RV-B14, H1-HeLa cells were used; for RV-C15, HeLa-E8 cells were used. The day of the experiment, the cells (~90% confluency) were infected at an MOI of 0.1 (low-MOI) and an MOI of 20 (high-MOI). The infected cells were incubated for an hour at 34° C./5% CO2. Following the incubation, the virus was removed, the cells were washed with 1x PBS, and then fresh infection media (DMEM, 2% FBS, 1% penicillin/streptomycin) was added. Cells were incubated at 34° C./5% CO₂ until complete cytopathic effect (CPE) was observed. Upon CPE, the virus was harvested by three repeated freeze-thaw cycles, and the cellular debris was removed by centrifugation (17,000 x g for 10 min at 4° C.). The supernatant was used to passage the virus into fresh H1-HeLa cells. This process was repeated until passage 10.

FIG. 43. Passaging RV-A01a in H1-HeLa cells at high titers. (A) Major deletion cluster forms within the P1 (structural) coding region and defective viral genomes (DVGs) within this cluster are all in-frame. The graph represents the start vs. stop position of the identified deletions. Depending on the size of the deletions, the data was split into in-frame (left; red circles) and out-of-frame (right; cyan circles) deletions. Pooled data from all passages (P01-P10) and all replicates (n = 5). (B) Appearance of deletions as a function of the passage number (P01-P10). Each panel represents the pooled data from all replicates (n = 5). The top-row panels represent the in-frame deletions, whereas the bottom-row panels represent the out-of-frame deletions across the genome.

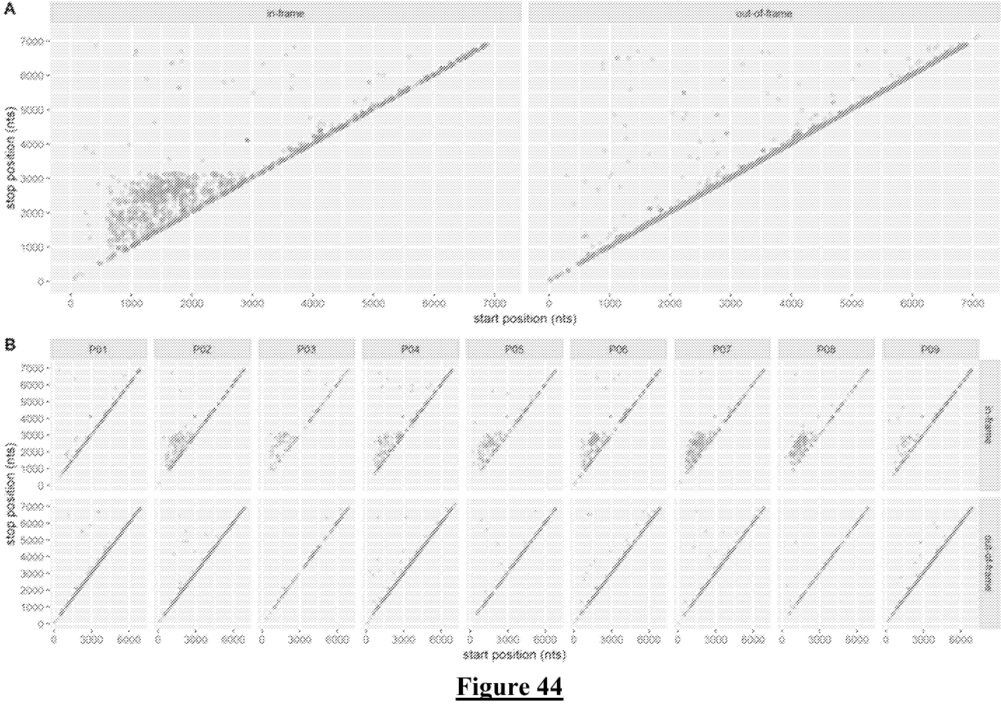

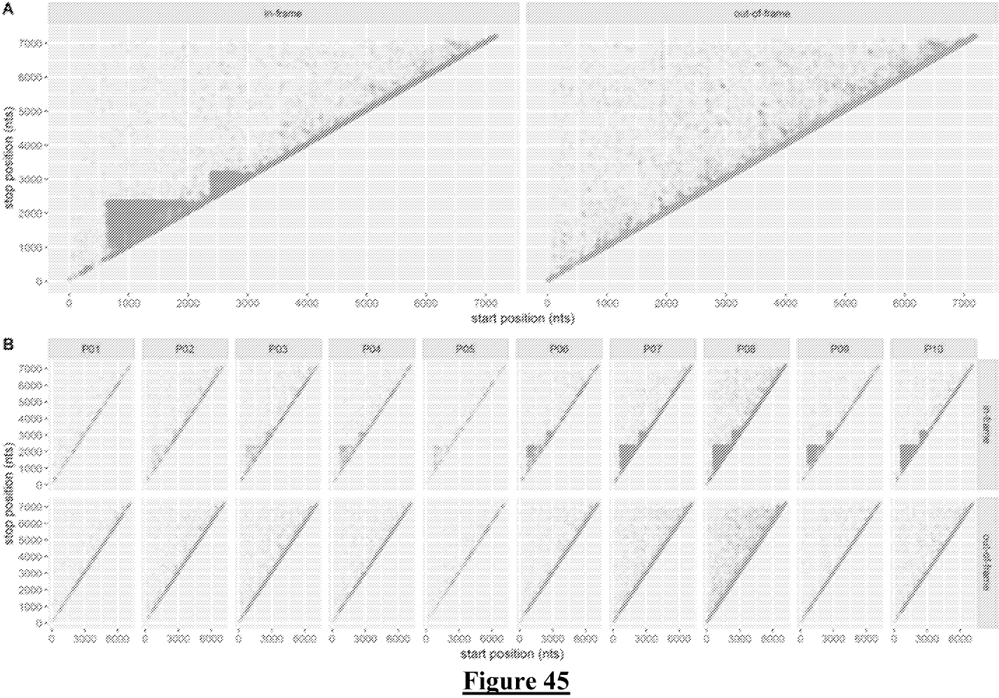

FIG. 44. Passaging RV-A16 in H1-HeLa cells at high titers. (A) Major deletion cluster forms within the P1 (structural) coding region and defective viral genomes (DVGs) within this cluster are all in-frame. The graph represents the start vs. stop position of the identified deletions. Depending on the size of the deletions, the data was split into in-frame (left; red circles) and out-of-frame (right; cyan circles) deletions. Pooled data from all passages (P01-P09) and all replicates (n = 5). (B) Appearance of deletions as a function of the passage number (P01-P09). Each panel represents the pooled data from all replicates (n = 5). The top-row panels represent the in-frame deletions, whereas the bottom-row panels represent the out-of-frame deletions across the genome.

FIG. 45. Passaging RV-B14 in H1-HeLa cells at high titers. (A) Major deletion clusters forms within the P1 (structural) coding region and defective viral genomes (DVGs) within these clusters are all in-frame. The graph represents the start vs. stop position of the identified deletions. Depending on the size of the deletions, the data was split into in-frame (left; red circles) and out-of-frame (right; cyan circles) deletions. Pooled data from all passages (P01-P10) and all replicates (n = 6). (B) Appearance of deletions as a function of the passage number (P01-P10). Each panel represents the pooled data from all replicates (n = 6). The top-row panels represent the in-frame deletions, whereas the bottom-row panels represent the out-of-frame deletions across the genome.

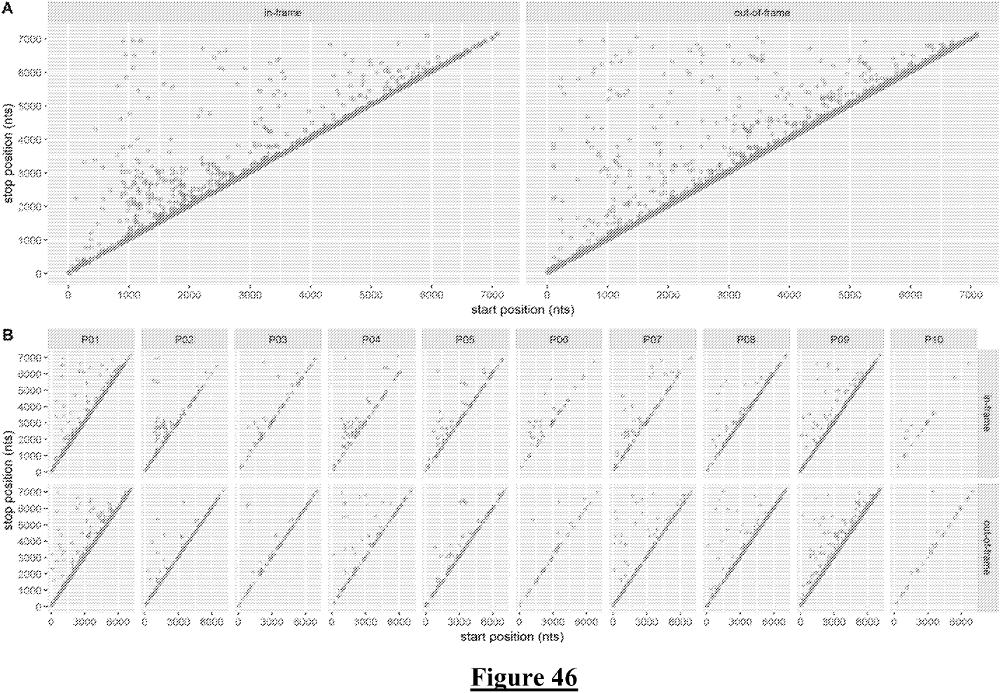

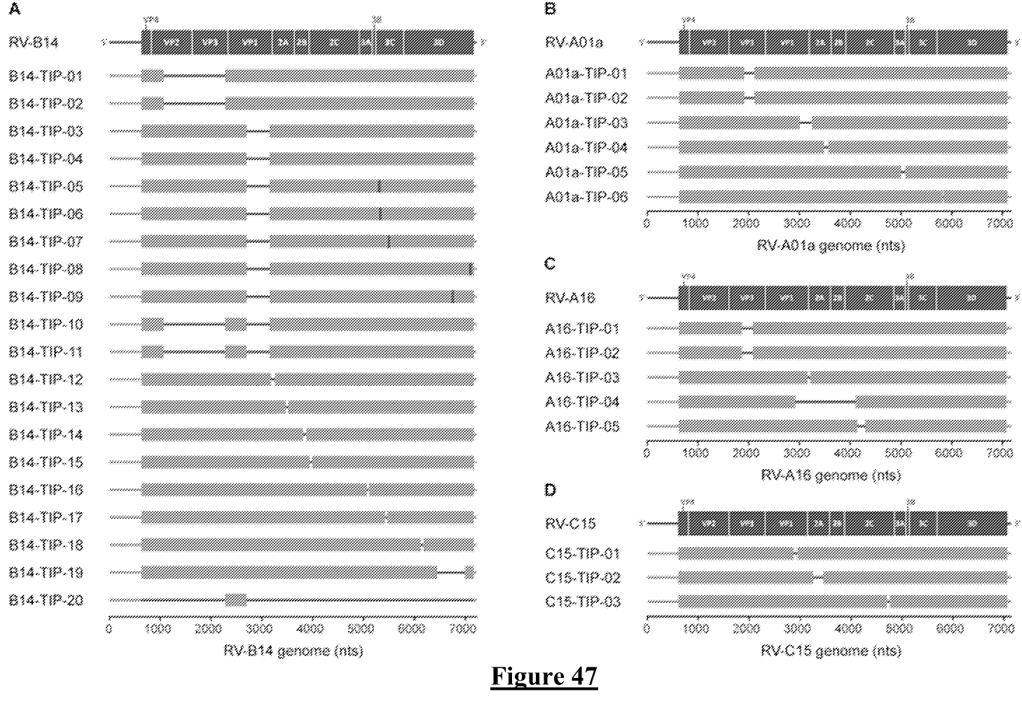

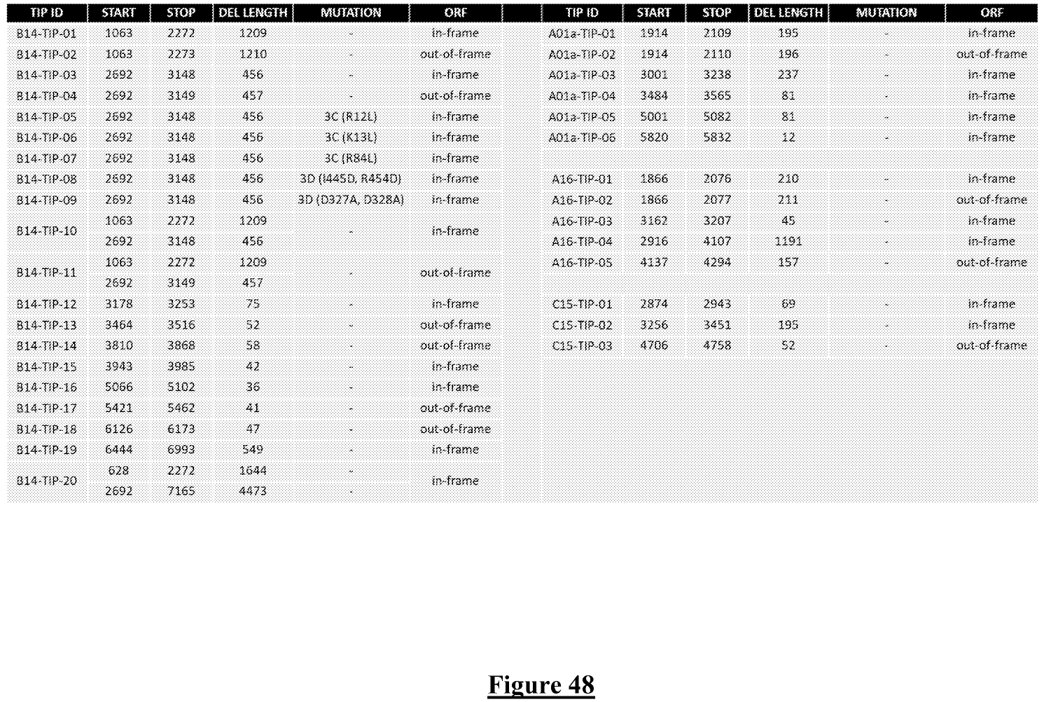

FIG. 46. Passaging RV-C15 in HeLa-E8 cells at high titers. (A) Major deletion clusters forms within the P1 (structural) coding region and defective viral genomes (DVGs) within these clusters are all in-frame. The graph represents the start vs. stop position of the identified deletions. Depending on the size of the deletions, the data was split into in-frame (left; red circles) and out-of-frame (right; cyan circles) deletions. Pooled data from all passages (P01-P10) and all replicates (n = 5). (B) Appearance of deletions as a function of the passage number (P01-P10). Each panel represents the pooled data from all replicates (n = 5). The top-row panels represent the in-frame deletions, whereas the bottom-row panels represent the out-of-frame deletions across the genome.

FIG. 47. Rhinovirus TIP candidates. (A) Rhinovirus (RV) -B14, (B) RV-A01a, (C) RV-A16 and (D) RV-C15 TIP candidates. The TIP candidates’ names include the RV type followed by the TIP number (e.g. B14-TIP-01). The deletions are indicated with horizontal red lines, whereas mutations are indicated with vertical red lines. On top of each figure panel the wild type genome for each viral type is presented (dark gray) with the location of the viral proteins indicated. The structural proteins in the same order as they appear: VP4, VP2, VP3, and VP1. Non-structural proteins in the same order as they appear: 2A, 2B, 2C, 3A, 3B, 3C, and 3D. The x-axis represents the viral genome length in nucleotides (nts).

FIG. 48. Details of Rhinovirus TIP candidates. Reported characteristics of each TIP candidate include: its ID, start and stop positions of the deletion, the length of the deletion (DEL LENGTH = STOP - START), mutations (if any), and whether the deletion keeps the open reading frame (ORF) in-frame or out-of-frame. If the deletion is divisible by 3 (remainder = 0), then it is in-frame, otherwise it is out-of-frame. Start and stop positions indicate the last remaining and the last deleted, respectively.

### Rhinovirus TIP Candidates Inhibit Wild-Type Virus In Vitro

Method: Prior to transfection, 96-well plates were seeded with 2.0 x 10⁴ H1-HeLa cells/well in complete media (DMEM, 10% FBS, 1% penicillin/streptomycin) and then incubated for 24 hrs at 37° C./5% CO₂ to reach ~90% confluency. The complete media was replaced with infection media (DMEM, 2% FBS, 1% penicillin/streptomycin) and the cells were co-transfected with wildtype and the selected TIP candidates at indicated molar ratios using the TransIT-mRNA transfection reagent. All the co-transfections were done in 1:1, 1:5, and 1:10 (WT:TIP) molar ratios and in triplicates (n = 3). In the case of WT RNA alone, the amount for “1” equals 25 ng of RNA. The transfected cells were incubated at 34° C./5% CO₂ for 48 hrs. Following the incubation, the virus was harvested by three repeated freeze-thaw cycles, and the cellular debris was removed by centrifugation (17,000 x g for 10 min at 4° C.). The titers of infectious virus were determined by plaque assay. As a control, WT RNA was co-transfected with control RNA (pTRI-Xef: Xenopus elongation factor 1α), which resulted in similar titers as WT alone. Additional controls, including the control RNA alone, TIP candidates alone, and a mock transfection were also tested, which yielded no infectious virus, as expected. Panrhinoviral activity of the best performing TIP candidate was tested the same way using WT RNA from RV-A01a and RV-A16.

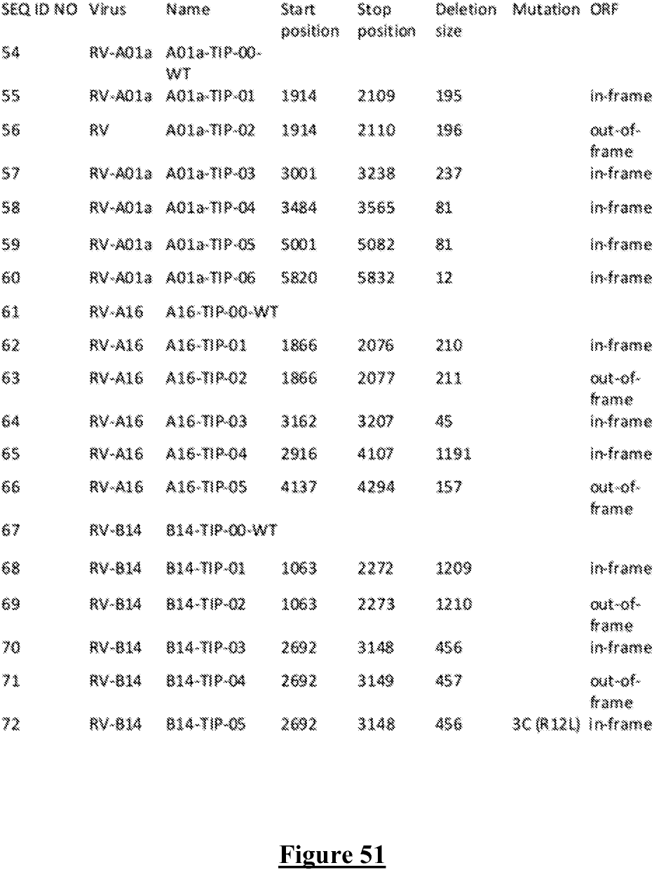

Results indicate that from this select set of TIP candidates, B14-TIP-03 candidate performed the best by lowering the WT titers by almost 100-fold (FIG. 49 A ). As a safety feature, we generated a non-replicative variant (K13L) of the B14-TIP-03 and renamed it as “B14-TIP-06”. The B14-TIP-06 has similar antiviral activity as the B14-TIP-03 (FIG. 49 B). Incorporating the same mutation in the context of WT RNA did not generate a viable virus, as expected (FIG. 49 B). These results have important implications in terms of safety of the use of DVGs as potential TIPs.

Our in vitro results indicate that antiviral feature of B14-TIP-03 is panrhinoviral. Co-transfecting B14-TIP-03 with RV-A01a and RV-A16 reduce infectious viral titers significantly. Further enforcing the potential of B14-TIP-03 to be used as a broad-spectrum antiviral against multiple RV types.

FIG. 49. A panrhinoviral TIP candidate reduces wildtype titers in vitro. (A) RV-B14-WT and TIP-candidate RNAs were co-transfected into H1-HeLa cells to assess the efficacy of a select set of TIP candidates (B14-TIP-01, B14-TIP-03, and B14-TIP-10). All the co-transfections were done in 1:1, 1:5, and 1:10 (WT:TIP) molar ratios. In the case of B14-WT alone, the amounts for “1” equals 25 ng of RNA. As controls, RV-B14-WT RNA was either transfected alone or with control RNA (pTRI-Xef: Xenopus elongation factor 1^{α}). Additional controls, including the control RNA alone, TIP candidates alone, and a mock transfection were also tested, which yielded no infectious virus, as expected. (B) The non-replicative variant of B14-TIP-03 (K13L variant - within the 3C protein) TIP was constructed and renamed as “B14-TIP-06”. The B14-TIP-06 TIP candidate has similar antiviral activity as the B14-TIP-03 TIP candidate. Introducing the same mutation in the context of the WT genome was not viable, as expected. (C) Panrhinoviral activity of B14-TIP-03 was tested by co-transfecting the TIP candidate with RV-B14, RV-A01a, and RV-A16 wildtype genomes. The x-axis represents the WT:TIP samples tested as well as their molar ratios in parentheses, and the y-axis represents Log₁₀(PFU/mL). Each condition was tested at least in triplicates (n ≥ 3).

### Rhinovirus TIP Candidate Is a Potent Stimulator of Antiviral Innate Immunity In Vitro

Method : Prior to transfection, 96-well plates were seeded with 2.0 x 10⁴ H1-HeLa cells/well in complete media (DMEM, 10% FBS, 1% penicillin/streptomycin) and then incubated for 24 hrs at 37° C./5% CO₂ to reach ~90% confluency. The complete media was replaced with infection media (DMEM, 2% FBS, 1% penicillin/streptomycin) and the cells were co-transfected with WT and B14-TIP-03 at a 1:10 (WT:TIP) molar ratio using the TransIT-mRNA transfection reagent. The amount for “1” equals 25 ng of WT RNA. All the co-transfections were in triplicates (n = 3). The transfected cells were incubated at 34° C./5% CO₂ for 3 hrs and then washed with thee times with phosphate buffered saline solution in order to remove the input RNA and prevent it from interfering with RT-qPCR experiments. Then, the transfected cells were incubated at 34° C./5% CO₂ for 48 hrs. Following the incubation, the virus was harvested by three repeated freeze-thaw cycles, and the cellular debris was removed by centrifugation (17,000 x g for 10 min at 4° C.). To isolate the viral RNA, the Direct-zol RNA Miniprep Kit (Zymo Research) was used following the manufacturer’s recommended protocol. The total RNA was then reverse transcribed using SuperScript IV (ThermoFisher) reverse transcriptase enzyme and random hexamer primers following manufacturer’s recommended protocol. Then, 2 uL of 10-fold diluted cDNA was amplified using Power SYBR™ Green PCR Master Mix using gene-specific primers against retinoic acid-inducible gene I (RIG-I), melanoma differentiation-associated gene 5 (MDA5), interferon (IFN)- α1, IFN- β1, and IFN-λ1 genes. The relative fold-change calculations were done using the ΔΔCₜ analysis against mock transfected cells with glyceraldehyde 3-phosphate dehydrogenase (GAPDH) housekeeping gene as a control.

Results : Based on qPCR results, B14-TIP-03 is a potent stimulator of antiviral innate immunity. The co-transfection of RV-B14-WT and control RNA (pTRI-Xef: Xenopus elongation factor 1^{α}) resulted in mild induction of all the genes tested. On the other hand, the co-transfection of RV-B14-WT and B14-TIP-03 RNAs resulted in a significant increase in the gene expression of pattern recognition receptors (PRRs), such as RIG-I and MDA5. Additionally, the B14-TIP-03 also induced the gene expression of IFN-β1 and IFN-λ1, but not IFN-α1. These results indicate the major mechanism of action for the B14-TIP-03.

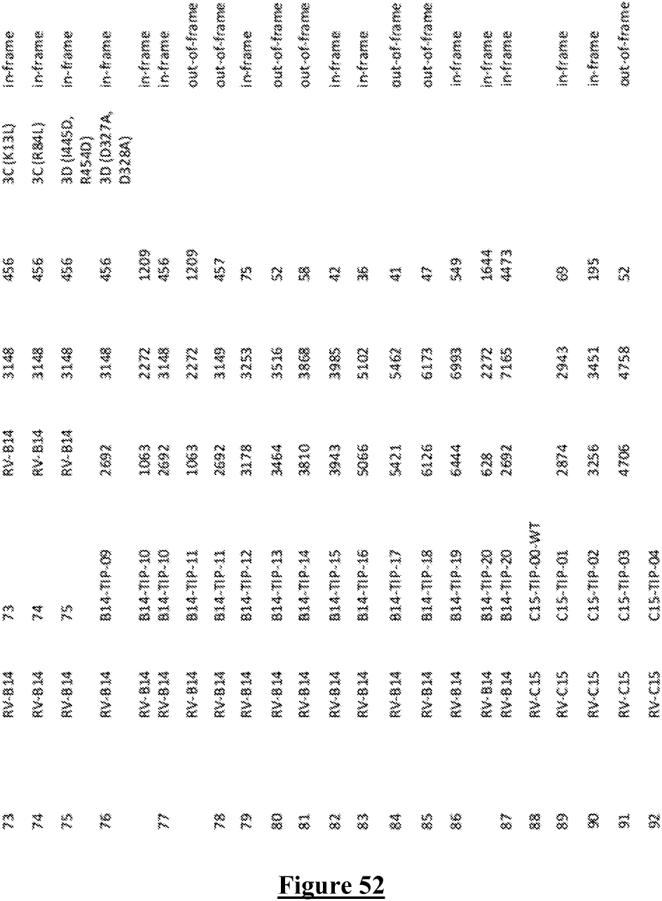

FIG. 50. The TIP candidate B14-TIP-03 is a potent stimulator of antiviral innate immunity. The fold change values are presented above each bar. All the co-transfections were done in 1:10 (WT:TIP) molar ratio. In the case of B14-WT alone, the amounts for “1” equals 25 ng of RNA. As a control, RV-B14-WT RNA was transfected with control RNA (pTRI-Xef: Xenopus elongation factor 1^{α}). The x-axis represents the WT:TIP samples tested, and the y-axis represents Log₁₀(PFU/mL). Each condition was tested in duplicates.

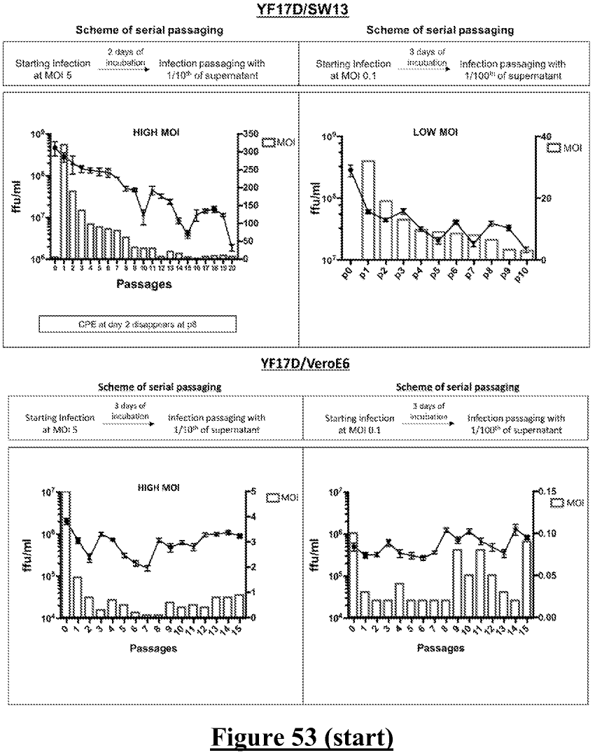

The identified DVGs are listed in FIGS. 51 and 52.

RV DVGs Sequences are selected from the group consisting of: SEQ ID NO: 55 (RV DVG A01a-TIP-01), SEQ ID NO: 56 (RV DVG A01a-TIP-02), SEQ ID NO: 57 (RV DVG A01a-TIP-03), SEQ ID NO: 58 (RV DVG A01a-TIP-04), SEQ ID NO: 59 (RV DVG A01a-TIP-05), SEQ ID NO: 60 (RV DVG A01a-TIP-06), SEQ ID NO: 62 (RV DVG A16-TIP-01), SEQ ID NO: 63 (RV DVG A16-TIP-02), SEQ ID NO: 64 (RV DVG A16-TIP-03), SEQ ID NO: 65 (RV DVG A16-TIP-04), SEQ ID NO: 66 (RV DVG A16-TIP-05), SEQ ID NO: 89 (RV DVG C15-TIP-01), SEQ ID NO: 90 (RVDVG C15-TIP-02), SEQ ID NO: 91 (RVDVG C15-TIP-03), SEQ ID NO: 68 (RV DVG B14-TIP-01), SEQ ID NO: 69 (RV DVG B14-TIP-02), SEQ ID NO: 70 (RV DVG B14-TIP-03), SEQ ID NO: 71 (RVDVG B14-TIP-04), SEQ ID NO: 72 (RV DVG B14-TIP-05), SEQ ID NO: 73 (RV DVG B14-TIP-06), SEQ ID NO: 74 (RV DVG B14-TIP-07), SEQ ID NO: 75 (RV DVG B14-TIP-08), SEQ ID NO: 76 (RV DVG B14-TIP-09), SEQ ID NO: 77 (RV DVG B14-TIP-10), SEQ ID NO: 78 (RV DVG B14-TIP-11), SEQ ID NO: 79 (RV DVG B14-TIP-12), SEQ ID NO: 80 (RV DVG B14-TIP-13), SEQ ID NO: 81 (RV DVG B14-TIP-14), SEQ ID NO: 82 (RV DVG B14-TIP-15), SEQ ID NO: 83 (RV DVG B14-TIP-16), SEQ ID NO: 84 (RV DVG B14-TIP-17), SEQ ID NO: 85 (RV DVG B14-TIP-18), SEQ ID NO: 86 (RV DVG B14-TIP-19) and SEQ ID NO: 87 (RV DVG B14-TIP-20).

Preferred RV DVGs sequences are selected from the group consisting of: SEQ ID NO: 55 (RV DVG A01a-TIP-01), SEQ ID NO: 62 (RV DVG A16-TIP-01), SEQ ID NO: 89 (RV DVG C15-TIP-01), SEQ ID NO: 68 (RV DVG B14-TIP-01), SEQ ID NO: 70 (RV DVG B14-TIP-03), SEQ ID NO: 73 (RV DVG B14-TIP-06) and SEQ ID NO: 77 (RV DVG B14-TIP-10).

Most preferred RV DVGs sequences are selected from the group consisting of: SEQ ID NO: 70 (RV DVG B14-TIP-03) and SEQ ID NO: 73 (RV DVG B14-TIP-06).

### Example 6: Yellow Fever Virus (YFV)

Generation and characterization of DVGs occurring within yellow fever virus passaging

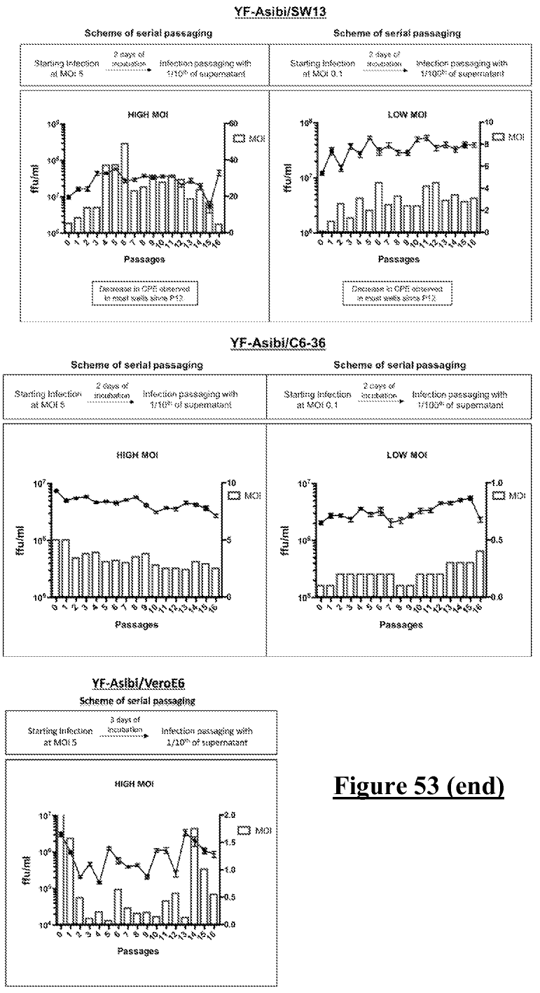

Yellow Fever virus Asibi (YF-Asibi) strain and vaccine (YF-17D) strain were blindly passaged between 10 and 20 times in 12 biological replicates at high and low MOI in mammalian (SW13, Vero) and mosquito (C6/36) cell lines. FIG. 53 represents the titration, by focus forming assay method, of samples from all the passaging performed. The graphs show both virus titer, expressed in ffu/ml, in logarithmic scale, and MOI value for each passage. From each experiment 4 passages were selected for sequencing, chosen as the passage before a viral titer drop. Currently, samples from both high and low MOI infections of YF-Asibi and YF-17D in SW13 have been already deep sequenced, while the other samples are ready for the library preparation.

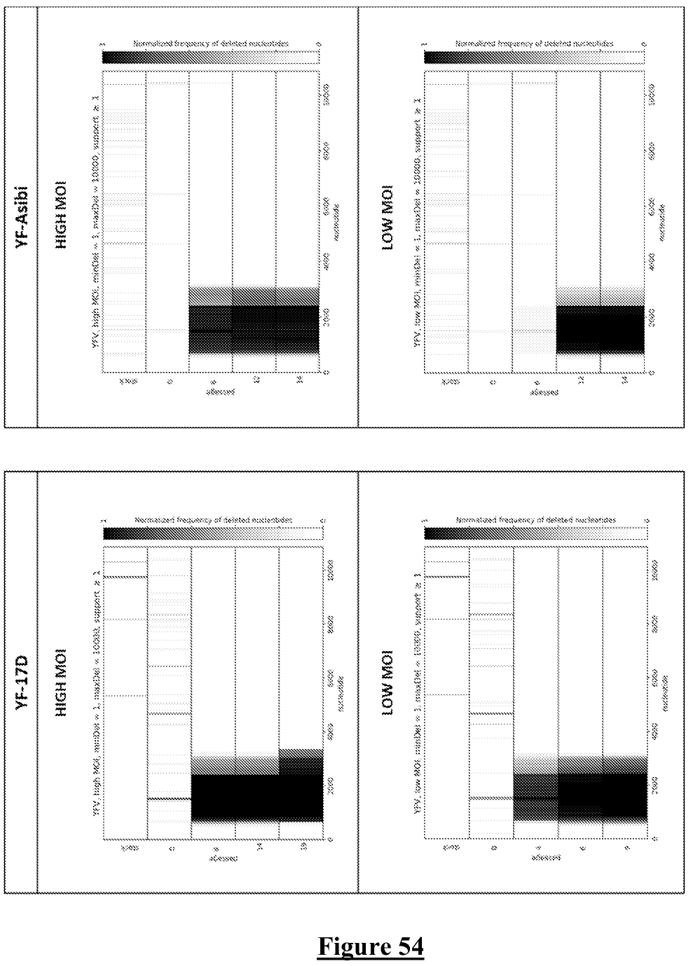

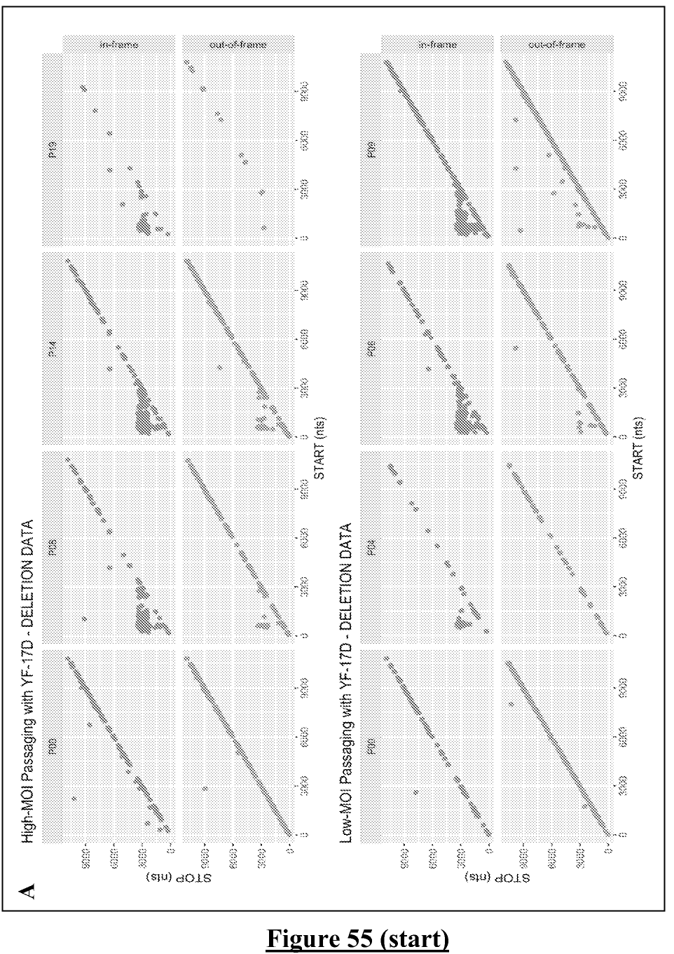

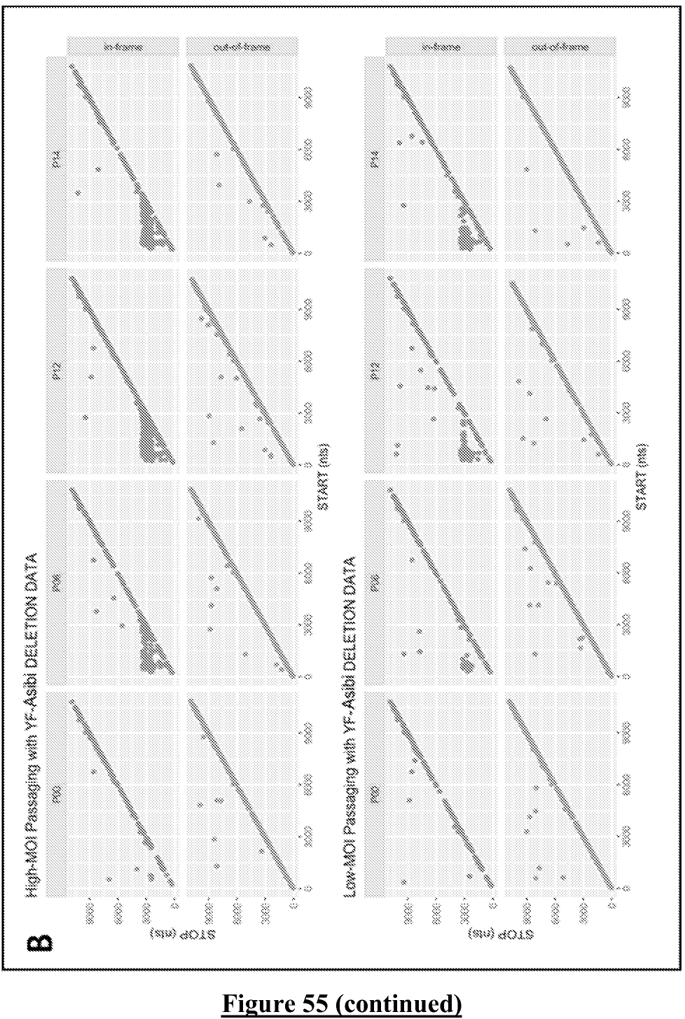

Computational analysis on sequenced passages identified all deletion hotspots across the genome and overall DVGs occurring within the population. FIG. 54 is a deletion heatmap resulting from the sequencing of passages of YF-17D and YF-Asibi at high and low MOI in SW13 cells. Each panel represents a different MOI, while each row is a different passage. The data shown here combines all biological replicates together. The nucleotide position is represented across the x-axis and spans the entire genome. Analysis reveals deletion hotspots are common to and conserved during the passaging, suggesting these DVGs are of higher fitness and can be packaged to infect new cells. Analysis of these data reveals deletion hotspots that are common to both high and low MOI. Moreover, some deletion hotspots are common to both viral strains. In FIG. 55 a further comparison of the DVGs found in the two YF strains is shown. The figure highlights a cluster of deletions in both virus strains that emerges and is conserved as the passage series progresses (suggesting high fitness of these DVGs).

The most conserved DVGs, schematically represented in Figure XX4, have been selected for de-novo synthesis of TIPs.

FIG. 53: Titration of blind passages. Each panel shows the samples collected during serial blind passagings of YF-17D and YF-Asibi on different cell lines. Samples were titrated using the focus forming assay (FFA) and here expressed as mean of the 12 replicates of each passage, in focus forming units per ml (ffu/ml). MOI for the calculated infection of each passage is showed as a mean of the 12 replicates (red bars).

FIG. 54: DVG identification in SW13 cells. YF-Asibi and YF-17D populations from selected passages were deep sequenced (RNAseq) and the normalized frequency of each deleted nucleotide position (x axis) throughout the YF genome is shown as a heat map. Each row within each panel represents a different passage. Each panel represents a different passage number for high and low MOI replicates for the two YF strains used.

FIG. 55: Identification of YF DVG hotspots in SW13 cells. Start (x axis) and stop (y axis) positions of the deletions were plotted for SW13 cell passages with YF-17D (A) and YF-Asibi (B). In frame (orange) and out of frame (cyan) deletions are showed for each passage analyzed. A clear hotspot was observed in the region with start and stop positions at approximately 500-3000nt start-stop, for both virus strains.

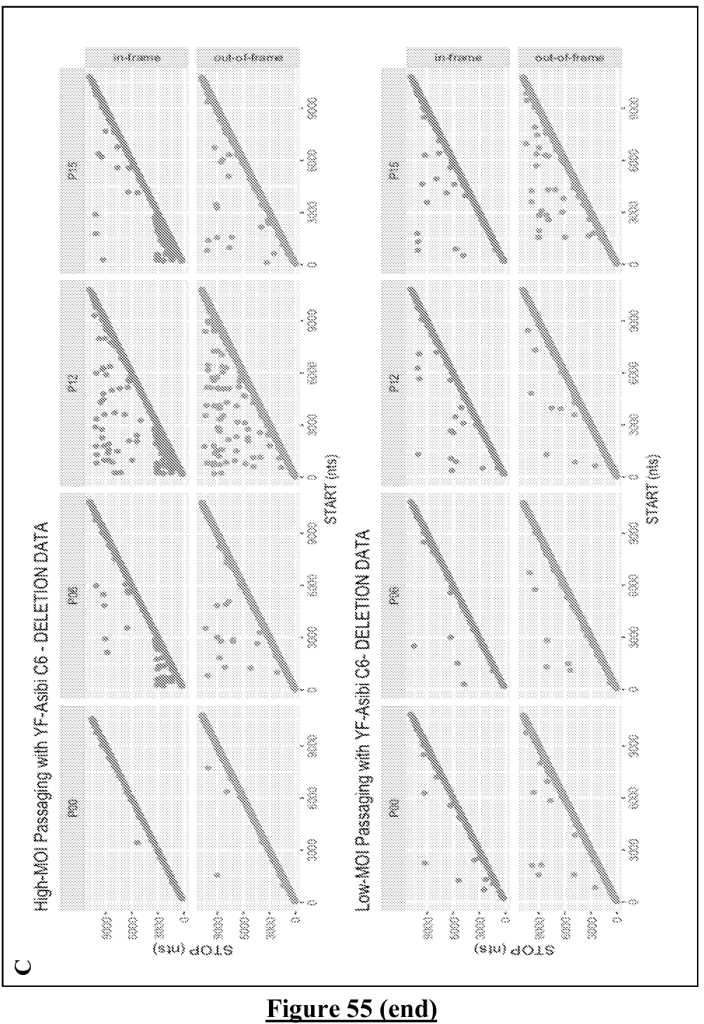

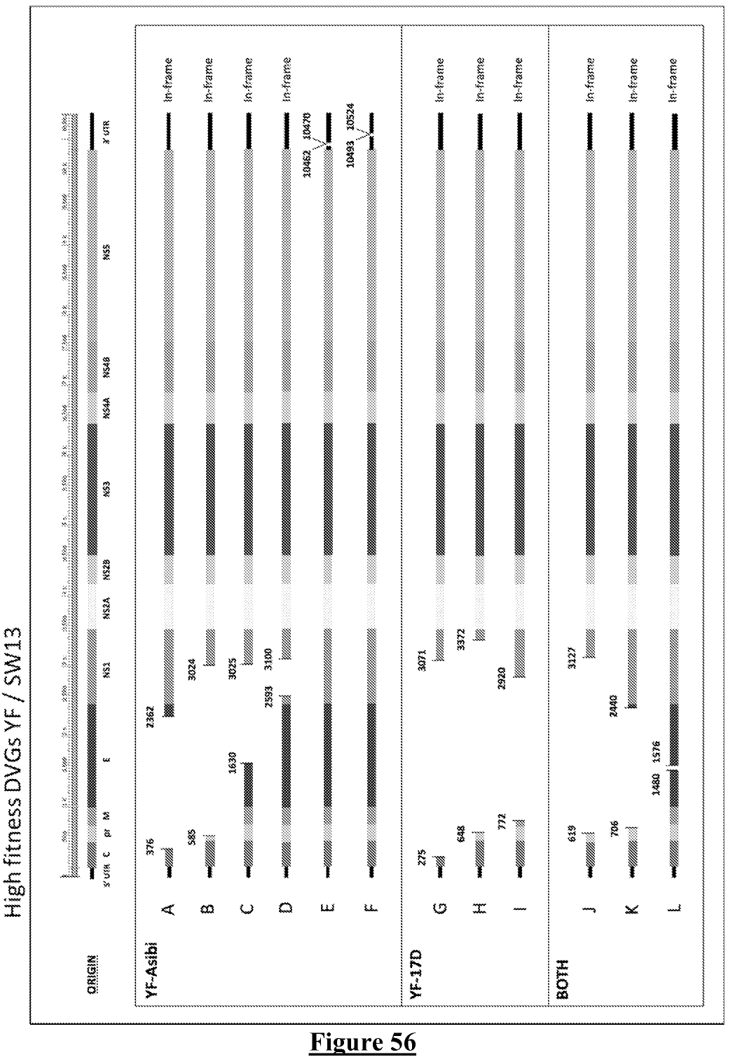

FIG. 56: Schematic representation of candidate YF DVGs. YFV candidate DVGs were chosen based on their conservation and frequency throughout passaging. The origin of each DVG is shown on the left side. Each letter on the left represents the provisional name of the DVG.

YFV DVGs sequences are selected from the group consisting of: SEQ ID NO:93 (YFV DVG-A), SEQ ID NO: 94 (YFV DVG-B), SEQ ID NO: 95 (YFV DVG-C), SEQ ID NO: 96 (YFV DVG-D), SEQ ID NO: 97 (YFV DVG-E), SEQ ID NO: 98 (YFV DVG-F), SEQ ID NO: 99 (YFV DVG-G), SEQ ID NO: 100 (YFV DVG-H), SEQ ID NO: 101 (YFV DVG-I), SEQ ID NO: 102 (YFV DVG-J), SEQ ID NO: 103 (YFV DVG-K) and SEQ ID NO: 104 (YFV DVG-L).

### Example 7: West Nile Virus (WNV)

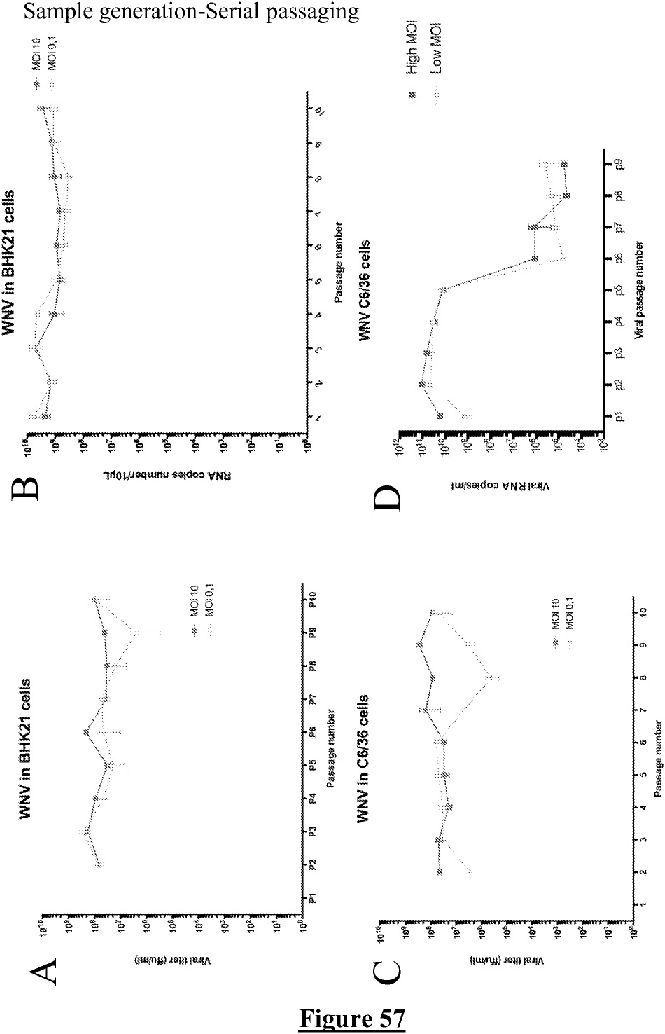

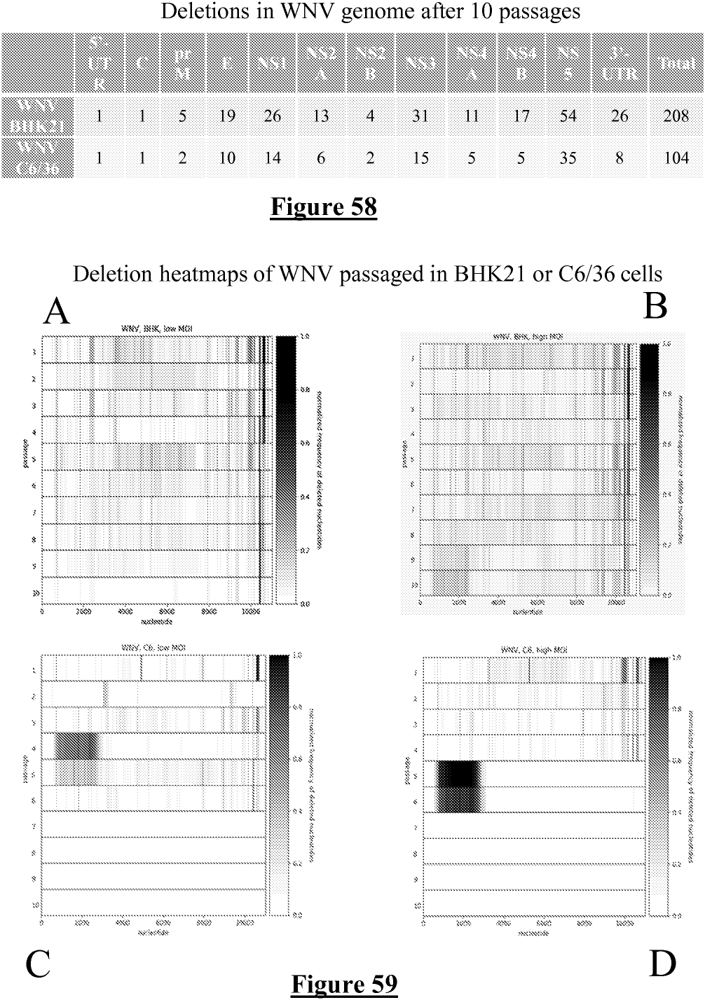

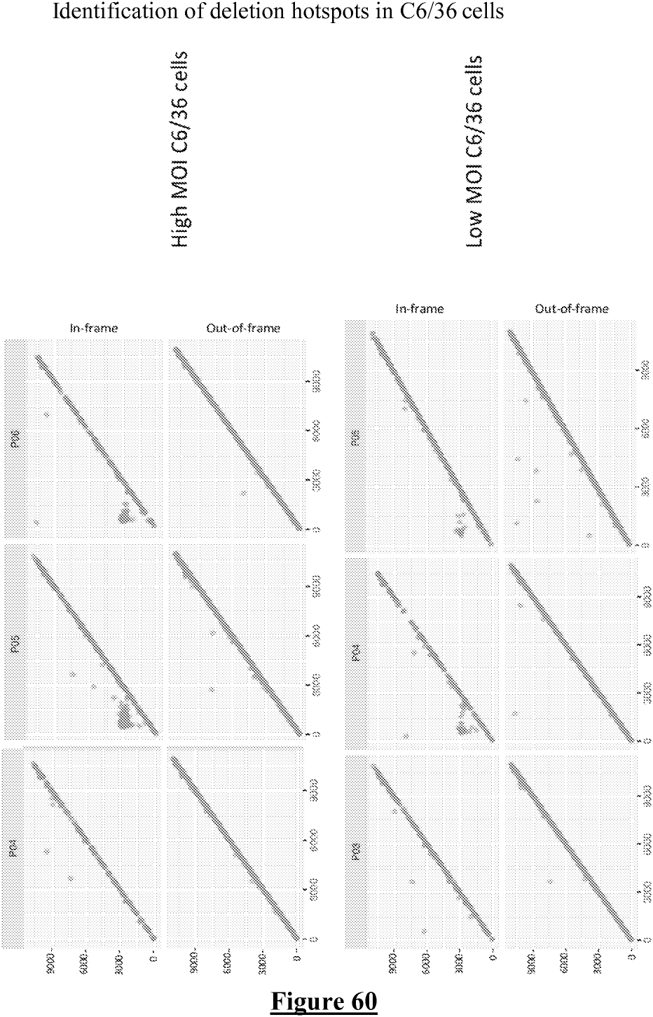

West Nile virus Israel 1998 strain (WNV) was passaged at a high multiplicity of infection in BHK or C6/36 cells. Briefly, cells that were seeded to reach between 70-80% confluence were infected with an initial MOI of either 0.1 or 10 PFU/cell. Two days post infection, 3 µl or 300 µl of the infected cell supernatant (respectively) was used for infection in the subsequent passage. The passaging was repeated 10 times. FIG. 57 represents the titration (by focus forming assay method) as well as RNA quantitation (by RT-QPCR) of samples from all passages performed in BHK21 cells (FIGS. 57 A and B) and C6/36 (FIGS. 57C and D). The RNA extracted from the samples was also used for next-generation sequencing. Libraries prepared using the RNA Library Prep kit (Illumina) were loaded on an Illumina NextSeq500 sequencer, using 150 cycle single-end reads. The reads obtained were analyzed using the computational pipeline described above. Computational analysis on sequenced passages identified all deletion hotspots across the genome and overall DVGs occurring within the population. Analysis of data from at least 2 reads with at least 10 deleted nt. in at least 5 samples after 10 passages in BHK21 or C6/36 cells reveals that the deletion hotspots are scattered through the whole genome in both cell lines, but are more numerous in BHK21 cells (FIG. 58). FIG. 59 shows the deletion heatmaps resulting from the sequencing of passages of WNV at low and high MOI in BHK21 (FIGS. 59A and B ) and C6/36 (FIGS. 59C and D). Each row in each panel represents a different passage number. Analysis reveals deletion hotspots are common to and conserved during the passaging in mosquito cells (C6/36), suggesting these DVGs are of higher fitness and can be packaged to infect new cells, while deletion hotspots are much less defined for mammalian cells (BHK21). Certain deletion hotspots are unique to the C6/36 cell line and take over other deletions in terms of frequency, such as the deletion hotspot located in and around the E glycoprotein (FIGS. 59C and D). This cluster of deletions emerges earlier at low MOI (FIG. 59C, passage 4) that high MOI (FIG. 59D, passage 5) and is conserved at the next passage for both MOI. However, due to a drop in RNA after passage 5 at both MOI in C6/36 cells (see FIG. 57D) no deletion heatmap could be generated after passage 6. Hotspots deletions for C6/36 cell passages 4, 5 and 6 (high MOI) and 3, 4 and 5 (low MOI) were identified by plotting the start and stop positions of the deletions (FIG. 60). As already predicted by the heatmaps (see FIG. 59), a clear hotspot was observed in the region with start and stop positions at approximately 500-3000 nt start-stop for both MOI, at passage 3 for the low MOI, and at passage 4 for the high MOI, but only in-frame for both MOI (FIG. 60, compare the orange and blue dots).

Altogether, the data show the importance of using more than one cell line or host to determine the total breadth and overall fitness of DVGs in different conditions. These data highlights that there are also many differences between cell types, yet reveals a cluster of deletions that emerges as the passage series progresses (suggesting high fitness of these DVGs).

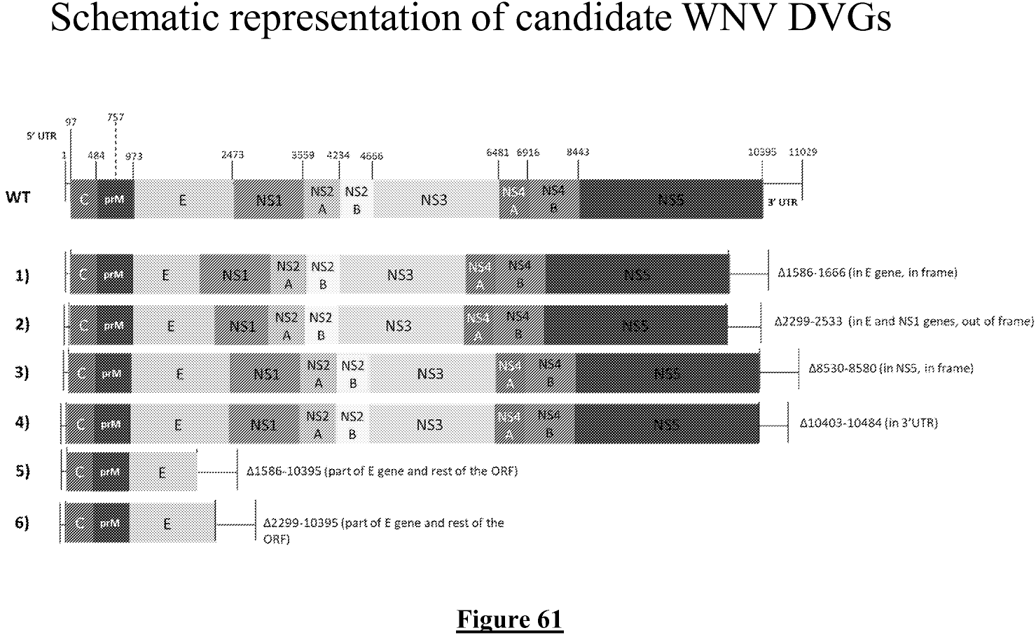

From these and other analyses, we selected a list of DVG candidates. The candidates, named numbered from 1 to 6, are shown in FIG. 61. The candidates span a variety of sizes and the deletions occur in different places in the genome, in and out-frame, representing a wider array of DVG candidates than could be identified using conventional passaging and isolation.

FIG. 57: Sample generation-Serial passaging. Each panel shows the samples collected during serial passaging of WNV on different cell lines. Samples were titrated using the focus forming assay (FFA) and here expressed as mean of the 3 replicates (and standard deviation) for each passage (x axis) in focus forming units per ml (ffu/ml; y axis). Multiplicity of infection (MOI) of 0.1 (light green) or 10 (dark green) is presented for BHK21 (panel A) and C6/36 cells (panel C). RNA quantitation was performed after RNA extraction from the cleared supernatant with Total RNA isolation Nucleospin kit. It is expressed as mean of the 3 replicates for each passage (x axis) in number of viral RNA copy per ml (viral RNA copy/ml; y axis) and presented for BHK21 (panel C) and C6/36 cells (panel D) for MOI of 0.1 (light green) or 10 (dark green).

FIG. 58: Deletion in WNV genome after 10 passages Analysis of data from at least 2 reads with at least 10 deleted nucleotides in at least 5 samples after 10 passages in BHK21 or C6/36 cells. Number and positions of the deletions are presented.

FIG. 59: Deletions heatmaps of WNV passaged in BHK21 or C6/36 cells WNV populations from the 10 passages in BHK21(panel A and B) or in C6/36 (panel C and D) cells were deep sequenced (RNAseq) and the normalized frequency (y axis) of each deleted nucleotide position (x axis) throughout the genome is shown as a heat map. Each row within each panel represents a different passage. Passages at high and low MOI are presented for each cell type.

FIG. 60: Identification of hotspot deletions in C6/36 cells Start (x axis) and stop (y axis) positions of the deletions were plotted for C6/36 cell passages with WNV. In frame (orange) and out of frame (cyan) deletions are showed for passages 4, 5 and 6 (high MOI) and 3, 4 and 5 (low MOI). A clear hotspot is observed in the region with start and stop positions at approximately 500-3000 nt start-stop for both MOI.

FIG. 61: Schematic representation of candidate WNV DVGs. WNV candidate DVGs were chosen based on their conservation and frequency throughout passaging in C6/36 cells. The WT genome is presented at the top, with encoded protein C (purple), prM (bleu), E (cyan), NS1 (dark green), NS2A (light green), NS2B (yellow), NS3 (orange), NS4A (brown), NS4B (pink) and NS5 (red), as well as 5′ and 3′ UTR (black line), and nucleotide numbering is indicated. Candidate DVGs are numbered from 1 to 6, and for each, location of the corresponding deletion is shown on the right. UTR: untranslated region; ORF: open reading frame.

WNV DVGs sequences are selected from the group consisting of: SEQ ID NO: 105 (WNV DVG-1), SEQ ID NO: 106 (WNV DVG-2), SEQ ID NO: 107 (WNV DVG-3), SEQ ID NO: 108 (WNV DVG-4), SEQ ID NO: 109 (WNV DVG-5) and SEQ ID NO: 110 (WNV DVG-6).

### EXAMPLE 7: Coronavirus (CV) - Identification of DVGs as Potential Antiviral Inhibitors Against SARS-CoV-2

Coronavirus SARS-CoV-2 strain (BetaCoV/ France/IDF0372/2020) was passaged at a high multiplicity of infection in Vero E6 cell. Briefly, cells were infected with an initial MOI of 5 x 10 \^5 PFU/cell. 3 days post infection, the infected cell supernatant was used for infection in the subsequent passage. The passaging was repeated 10 times. The overall process included passaging at high-MOI, followed by viral RNA purification, deep sequencing, and data analysis to identify defective viral genomes (DVGs).

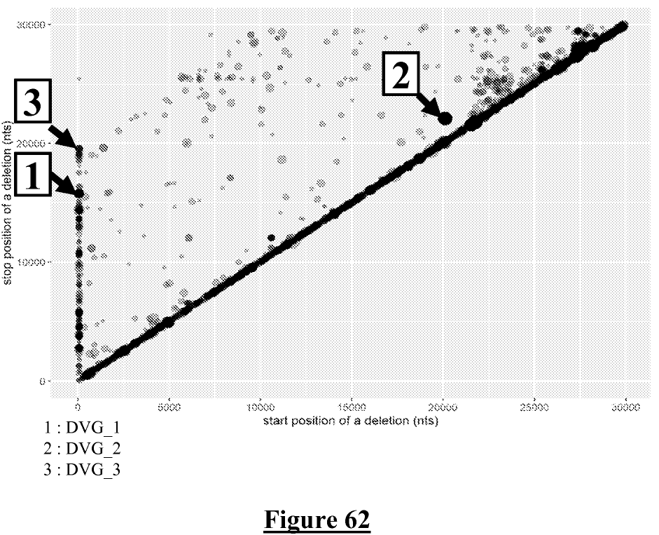

FIG. 62: Deep sequencing map of SARS-CoV-2 DVGs. 3 DVGs were chosen according to their abundance, maintenance in the cell culture upon passage, and size. These 3 DVGs were isolated and sequenced. SARS-CoV-2 DVGs 1, 2 and 3 have the nucleotide sequence of SEQ ID NO: 111, SEQ ID NO: 112 and SEQ ID NO: 113. One of these SARS-CoV-2 DVG was selected (SARS-CoV-2 DVG_2 having the nucleotide sequence of SEQ ID NO/ 112) for further analysis.

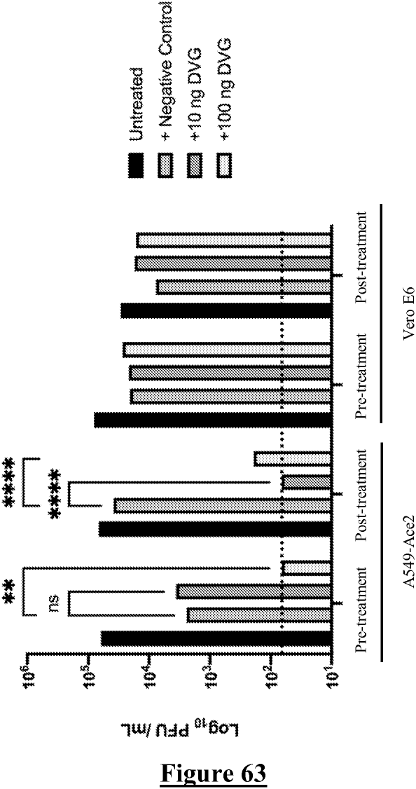

FIG. 63: competition assay in Vero E6 cells and in A549-Ace2 cells (Homo sapiens, epithelial, lung carcinoma) . SARS-CoV-2 DVG_2 was cloned in a vector under the control of a T7 promotor. RNA corresponding to the SARS-CoV-2 DVG_2 clone was synthetized following the procedure indicated for the mMESSAGE mMACHINE T7 Transcription Kit (Thermofisher). RNA of the SARS-CoV-2 DVG_2 clone was transfected into Vero E6 cells or A549-Ace2 cells 4 hours either before or after infection of the cells with wilt type SARS-CoV-2 virus. As a control, a small RNA of the same size as the RNA of the SARS-CoV-2 DVG_2 clone was transfected in the same conditions. Results were obtained by plaque assay. As illustrated on FIG. 63, the transfection of the RNA control does not impact SARS-CoV-2 replication capability, as compared to untreated cells, both in Vero E6 cells and in A549-Ace2 cells (see the two columns on the left of each graph of FIG. 63). The transfection of the RNA of the SARS-CoV-2 DVG_2 clone does not have an impact on the replication of SARS-CoV-2 virus in Vero E6 cells. On A549-Ace2 cells, the replication capability of SARS-CoV-2 virus is impacted by the transfection of the RNA of the SARS-CoV-2 DVG_2 clone, in particular when 100 ng of RNA are transfected within the cells. A reduction of 2.5 log of SARS-CoV-2 titers was observed by post-treating A549-Ace2 cells with 10 ng of the RNA of the SARS-CoV-2 DVG_2 clone. A reduction of 2.5 log of SARS-CoV-2 titers was observed by pre-treating or post-treating A549-Ace2 cells with 100 ng of the RNA of the SARS-CoV-2 DVG_2 clone.

SARS-CoV-2 sequences are selected from the group consisting of: SEQ ID NO:111 (SARS-CoV-2 DVG_1), SEQ ID NO: 112 (SARS-CoV-2 DVG_2) and SEQ ID NO: 113 (SARS-CoV-2 DVG_3).

### REFERENCES

1. Chretien, J.-P. et al. DROUGHT-ASSOCIATED CHIKUNGUNYA EMERGENCE ALONG COASTAL EAST AFRICA. 3.

2. Vignuzzi, M. & Higgs, S. The Bridges and Blockades to Evolutionary Convergence on the Road to Predicting Chikungunya Virus Evolution. Annu. Rev. Virol. 4, 181-200 (2017).

3. Levi, L. I. & Vignuzzi, M. Arthritogenic Alphaviruses: A Worldwide Emerging Threat? Microorganisms 7, 133 (2019).

4. Von Magnus, P. Incomplete forms of influenza virus. Advances in virus research (1954).

5. Rezelj, V. V., Levi, L. I. & Vignuzzi, M. The defective component of viral populations. Curr. Opin. Virol. 33, 74-80 (2018).

6. Vignuzzi, M. & López, C. B. Defective viral genomes are key drivers of the virus-host interaction. Nat. Microbiol. 1 (2019) doi:10.1038/s41564-019-0465-y.

7. Tapia, K. et al. Defective viral genomes arising in vivo provide critical danger signals for the triggering of lung antiviral immunity. PLoS Pathog. 9, e1003703 (2013).

8. Shirogane, Y. et al. Experimental and mathematical insights on the competition between poliovirus and a defective interfering genome. bioRxiv 519751 (2019) doi:10.1101/519751.

9. Poirier, E. Z. et al. Dicer-2-Dependent Generation of Viral DNA from Defective Genomes of RNA Viruses Modulates Antiviral Immunity in Insects. Cell Host Microbe 23, 353-365.e8 (2018).

10. Sun, Y. et al. Immunostimulatory Defective Viral Genomes from Respiratory Syncytial Virus Promote a Strong Innate Antiviral Response during Infection in Mice and Humans. PLOS Pathog. 11, e1005122 (2015).

11. Mura, M. et al. Nonencapsidated 5′ Copy-Back Defective Interfering Genomes Produced by Recombinant Measles Viruses Are Recognized by RIG-I and LGP2 but Not MDA5. J. Virol. 91, (2017).

12. Li, D. et al. Defective interfering viral particles in acute dengue infections. PloS One 6, e19447 (2011).

13. Vasilijevic, J. et al. Reduced accumulation of defective viral genomes contributes to severe outcome in influenza virus infected patients. PLOS Pathog. 13, e1006650 (2017).

14. Dimmock, N. J., Rainsford, E. W., Scott, P. D. & Marriott, A. C. Influenza Virus Protecting RNA: an Effective Prophylactic and Therapeutic Antiviral. J. Virol. 82, 8570-8578 (2008).

15. Dimmock, N. J. & Easton, A. J. Defective Interfering Influenza Virus RNAs: Time To Reevaluate Their Clinical Potential as Broad-Spectrum Antivirals? J. Virol. 88, 5217-5227 (2014).

16. Poirier, E. Z. et al. Low-Fidelity Polymerases of Alphaviruses Recombine at Higher Rates To Overproduce Defective Interfering Particles. J. Virol. 90, 2446-2454 (2015).

17. Coffey, L. L. & Vignuzzi, M. Host alternation of chikungunya virus increases fitness while restricting population diversity and adaptability to novel selective pressures. J. Virol. 85, 1025-1035 (2011).

18. Stapleford, K. A. et al. Whole-Genome Sequencing Analysis from the Chikungunya Virus Caribbean Outbreak Reveals Novel Evolutionary Genomic Elements. PLoS Negl. Trop. Dis. 10, e0004402 (2016).

19. Garcia-Nafria, J., Watson, J. F. & Greger, I. H. IVA cloning: A single-tube universal cloning system exploiting bacterial In Vivo Assembly. Sci. Rep. 6, (2016).

20. Zhang, X. et al. Differences in genome characters and cell tropisms between two chikungunya isolates of Asian lineage and Indian Ocean lineage. Virol. J. 15, 130 (2018).

21. Rezelj, V. V., Levi, L. I. & Vignuzzi, M. The defective component of viral populations. Curr. Opin. Virol. 33, 74-80 (2018).

22. Dimmock, N. J. In vivo antiviral activity: defective interfering virus protects better against virulent Influenza A virus than avirulent virus. J. Gen. Virol. 87, 1259-1265 (2006).

23. Mann, A. et al. Interfering vaccine (defective interfering influenza A virus) protects ferrets from influenza, and allows them to develop solid immunity to reinfection. Vaccine 24, 4290-4296 (2006).

24. Mercado-López, X. et al. Highly immunostimulatory RNA derived from a Sendai virus defective viral genome. Vaccine 31, 5713-5721 (2013).

25. Fisher, D. G., Coppock, G. M. & López, C. B. Virus-derived immunostimulatory RNA induces type I IFN-dependent antibodies and T-cell responses during vaccination. Vaccine 36, 4039-4045 (2018).

26. Martinez-Gil, L. et al. A Sendai virus-derived RNA agonist of RIG-I as a virus vaccine adjuvant. J. Virol. 87, 1290-1300 (2013).

27. Easton, A. J. et al. A novel broad-spectrum treatment for respiratory virus infections: influenza-based defective interfering virus provides protection against pneumovirus infection in vivo. Vaccine 29, 2777-2784 (2011).

28. Fuller, F. J. & Marcus, P. I. Interferon induction by viruses. IV. Sindbis virus: early passage defective-interfering particles induce interferon. J. Gen. Virol. 48, 63-73 (1980).

29. Johnston, M. D. The characteristics required for a Sendai virus preparation to induce high levels of interferon in human lymphoblastoid cells. J. Gen. Virol. 56, 175-184 (1981).

30. Marcus, P. I. & Sekellick, M. J. Defective interfering particles with covalently linked [+/-]RNA induce interferon. Nature 266, 815-819 (1977).

31. van den Hoogen, B. G. et al. Excessive production and extreme editing of human metapneumovirus defective interfering RNA is associated with type IIFN induction. J. Gen. Virol. 95, 1625-1633 (2014).

32. Yount, J. S., Gitlin, L., Moran, T. M. & Lopez, C. B. MDA5 Participates in the Detection of Paramyxovirus Infection and Is Essential for the Early Activation of Dendritic Cells in Response to Sendai Virus Defective Interfering Particles. J. Immunol. 180, 4910-4918 (2008).

33. Shivakoti, R., Siwek, M., Hauer, D., Schultz, K. L. W. & Griffin, D. E. Induction of dendritic cell production of type I and type III interferons by wild-type and vaccine strains of measles virus: role of defective interfering RNAs. J. Virol. 87, 7816-7827 (2013).

34. Meng, B. et al. Unexpected complexity in the interference activity of a cloned influenza defective interfering RNA. Virol. J. 14, 138 (2017).

35. Matusali, G. et al. Tropism of the Chikungunya Virus. Viruses 11, (2019).

36. Forrester, N., Coffey, L. & Weaver, S. Arboviral Bottlenecks and Challenges to Maintaining Diversity and Fitness during Mosquito Transmission. Viruses 6, 3991-4004 (2014).

37. Forrester, N. L., Guerbois, M., Seymour, R. L., Spratt, H. & Weaver, S. C. Vector-Borne Transmission Imposes a Severe Bottleneck on an RNA Virus Population. PLoS Pathog. 8, e1002897 (2012).

38. Stapleford, K. A. et al. Emergence and transmission of arbovirus evolutionary intermediates with epidemic potential. Cell Host Microbe 15, 706-716 (2014).

39. Weger-Lucarelli, J. et al. Using barcoded Zika virus to assess virus population structure in vitro and in Aedes aegypti mosquitoes. Virology 521, 138-148 (2018).

40. Cuevas, J. M., Duran-Moreno, M. & Sanjuan, R. Multi-virion infectious units arise from free viral particles in an enveloped virus. Nat. Microbiol. 2, 17078 (2017).

41. Sanjuan, R. Collective Infectious Units in Viruses. Trends Microbiol. 25, 402-412 (2017).

42. Leeks, A., Sanjuan, R. & West, S. A. The evolution of collective infectious units in viruses. Virus Res. 265, 94-101 (2019).

43. Acosta-Ampudia, Y. et al. Mayaro: an emerging viral threat? Emerg. Microbes Infect. 7, 163 (2018).

44. Rezza, G., Chen, R. & Weaver, S. C. O’nyong-nyong fever: a neglected mosquito-borne viral disease. Pathog. Glob. Health 111, 271-275 (2017).

45. Mackay, I. M. & Arden, K. E. Mayaro virus: a forest virus primed for a trip to the city? Microbes Infect.0 18, 724-734 (2016).

46. Esposito, D. L. A. & Fonseca, B. A. L. da. Will Mayaro virus be responsible for the next outbreak of an arthropod-borne virus in Brazil? Braz. J. Infect. Dis. Off. Publ. Braz. Soc. Infect. Dis. 21, 540-544 (2017).

47. Liu, X., Tharmarajah, K. & Taylor, A. Ross River virus disease clinical presentation, pathogenesis and current therapeutic strategies. Microbes Infect. 19, 496-504 (2017).

48. Pilot, R. et al. A Review on Surface-Enhanced Raman Scattering. Biosensors 9, 57 (2019).

49. Erasmus, J. H. et al. A Nanostructured Lipid Carrier for Delivery of a Replicating Viral RNA Provides Single, Low-Dose Protection against Zika. Mol. Ther. (2018) doi:10.1016/j.ymthe.2018.07.010.

50. Couderc, T. et al. Prophylaxis and therapy for Chikungunya virus infection. J. Infect. Dis. 200, 516-523 (2009).

51. Haese, N. N. et al. Animal Models of Chikungunya Virus Infection and Disease. J. Infect. Dis. 214, S482-S487 (2016).

52. Ferguson, N. M. et al. Modeling the impact on virus transmission of Wolbachia-mediated blocking of dengue virus infection of Aedes aegypti. Sci. Transl. Med. 7, 279ra37 (2015).

53. Ferguson, N. M. Challenges and opportunities in controlling mosquito-borne infections. Nature 559, 490-497 (2018).

1. A method for producing a defective interfering viral genome (DVG), the method comprising:
providing a first set of at least three replicate in vitro cell cultures, comprising a solid support and cells infected with a reference infectious virus at a high multiplicity of infection (MOI);
providing a second set of at least three replicate in vitro cell cultures, comprising a solid support and cells infected with the reference infectious virus at a low multiplicity of infection (MOI);
culturing the first and second sets of replicate in vitro cell cultures for at least 5 passages under mutagenic conditions;
collecting a plurality of DVG candidates from the medium of the first and second sets of in vitro replicate cell cultures;
deep sequencing the collected DVG candidates; and
selecting a DVG from the plurality of DVG candidates.

2. The method of claim 1, further comprising:
providing a third set of at least three replicate in vitro cell cultures, comprising a solid support and cells infected with a reference infectious virus at a high multiplicity of infection (MOI); and/or
providing a fourth set of at least three replicate in vitro cell cultures, comprising a solid support and cells infected with the reference infectious virus at a low multiplicity of infection (MOI); and
culturing the third and/or fourth sets of replicate in vitro cell cultures for at least 5 passages under non-mutagenic conditions.

3. The method of claim 1 or claim 2, wherein selecting a DVG from the plurality of DVG candidates comprises:
comparing the sequences of the DVG candidates with the sequence of the genome of the reference infectious virus;
identifying at least one DVG candidate having a genome comprising at least one splicing event where the splicing event is a deletion or a rearrangement;
determining the relative frequency of at least one DVG candidate having a genome with at least one splicing event among the population of sequenced DVG candidate genomes; and,
selecting at least one DVG for which:
a) at least one splicing event is more abundant at high MOI cultures than at low MOI cultures; and/or
b) at least one splicing event appears in at least 2, 3, 4, 5, 6, 7, 8 or 9 different cell lines; and/or,
c) at least one splicing event is found in at least 3 of at least 12, 24 or 36 independent replicates, after at least 5, 10, 15 or 20 passages; and/or,
d) at least one splicing event is a deletion event and the nucleotide size of the deletion is at least 40, 42, 50, 100, 150, 200, 400, 600 or 1000 nucleotides; and/or,
e) an open reading frame in the DVG is maintained; and/or,
f) the structural domains and functions necessary for viral replication, are maintained in the DVG; and/or
g) the DVG comprises at least one mutation that reduces the self-replicative capacity of the reference infectious virus.

4. The method according to any one of claims 1 to 3, wherein the DVG is characterized by an in vitro inhibitory activity of at least 50%, 60%, 70%, 80% or 90% against the reference infectious virus.

5. The method according to any one of claims 1 to 4, wherein the reference infectious virus has a mutator phenotype.

6. The method according to any one of claims 1 to 5, wherein the reference infectious virus is a Chikungunya virus (CHIKV), Zika virus (ZIKV), Enterovirus 71 (EV71), Rhinovirus (RV), Yellow Fever virus (YFV),West Nile Virus (WNV) or Coronavirus (CV).

7. The method according to any one of claims 1 to 5, wherein the reference infectious virus is selected from the group consisting of: CHIKV Indian Ocean lineage (GenBank accession no. AM258994), CHIKV Caribbean strain (GenBank accession no. LN898104.1), ZIKV African strain MR766 (KY989511.1), EV71 Sep006 (GenBank: KX197462.1), RV-A01a (NC_038311.1), RV-A16 (L24917.1), RV-B14 (L05355.1), RV-C15 (GU219984.1), West Nile virus Israel 1998 and YFV strain, Yellow Fever Virus Asibi (YF-Asibi) strain,Yellow Fever Virus vaccine (YF-17D) strain, and Coronavirus SARS-CoV-2 strain.

8. The method according to any one of claims 1 to 7, wherein the cells are a mammalian cell line selected from the group consisting of: a cell line derived from African green monkey kidney cells (optionally Vero, optionally Vero-E6 cell line), a cell line derived from human muscle (optionally RD cell line), a cell line derived from baby hamster kidneys (optionally BHK cell line), a cell line derived from human embryonic kidney cells (optionally HEK293T cell line) a cell line derived from liver carcinoma cells (optionally Huh7 cell line), and a cell line derived from cervical adenocarcinoma (optionally H1-HeLa, optionally HeLa-E8 cell line).

9. The method according any one of claims 1 to 7, wherein the cells are mosquito a cell line selected from the group consisting of: a cell line derived from Aedes aegypti (optionally Aag2 cell line), a cell derived from Aedes albopictus (optionally C6/36 cell line) and a cell line derived from Aedes albopictus (optionally U4.4 cell line).

10. The method according to any one of claims 1 to 9, wherein the low MOI for infection is from 0.001 to 0.1 PFU/cell, in particular from 0.01 to 0.1 PFU/cell.

11. The method according to any one of claims 1 to 10, wherein the high MOI for infection is from 1 PFU/cell to 100 PFU/cell, in particular from 5 to 50 PFU/cell or 5 to 20 PFU/cell.

12. A DVG produced by the method of any one of claims 1 to 11.

13. A DVG comprising or consisting of a nucleotide sequence selected from the group consisting of: SEQ ID NO: 1 (DG1 : EV71 DVG 293-390), SEQ ID NO: 2 (DG2 : EV71 DVG 294-404), SEQ ID NO: 3 (DG3 : EV71 DVG 1746-2895), SEQ ID NO: 4 (DG4 : EV71 DVG 1752-2894), SEQ ID NO: 5 (DG5 : EV71 DVG 1752-2896), SEQ ID NO: 6 (DG6 : EV71 DVG 1880-6487), SEQ ID NO: 7 (DG7 : EV71 DVG 1880-6488), SEQ ID NO: 8 (DG8 : EV71 DVG 3513-6516), SEQ ID NO: 9 (DG9 : EV71 DVG 5475-5634), SEQ ID NO: 10 (DG10: EV71 DVG 5610-6876), SEQ ID NO: 11 (DG11 : EV71 DVG 5610-6877), SEQ ID NO: 12 (DG12: EV71 DVG 5746-5821), SEQ ID NO: 13 (DG13 : EV71 DVG 6322-6356), SEQ ID NO: 14 (DG14: EV71 DVG 6322-6358), SEQ ID NO: 15 (DG15: EV71 DVG 6728-6779), SEQ ID NO: 16 (DG16: EV71 DVG 6966-7012), SEQ ID NO: 17 (DG17: EV71 DVG 6966-7014), SEQ ID NO: 18 (DG18: EV71 DVG 7098-7122), SEQ ID NO: 19 (DG19: EV71 DVG 7165-7200), SEQ ID NO: 20 (DG20: EV71 DVG 7165-7201), SEQ ID NO: 21 (DG21: EV71 DVG 7238-7292), SEQ ID NO: 22 (ZIKV DVG-A), SEQ ID NO: 23 (ZIKV DVG-B), SEQ ID NO: 24 (ZIKV DVG-C), SEQ ID NO: 25 (ZIKV DVG-D), SEQ ID NO: 26 (ZIKV DVG-E), SEQ ID NO: 27 (ZIKV DVG-F), SEQ ID NO: 28 (ZIKV DVG-G), SEQ ID NO: 29 (ZIKV DVG-H), SEQ ID NO: 30 (ZIKV DVG-I), SEQ ID NO: 31 (ZIKV DVG-J), SEQ ID NO: 32 (ZIKV DVG-K), SEQ ID NO: 33 (ZIKV DVG-L), SEQ ID NO: 34 (CHIKV DVG-IV1), SEQ ID NO: 35 (CHIKV DVG-IV2), SEQ ID NO: 36 (CHIKV DVG-IV3), SEQ ID NO: 37 (CHIKV DVG-IV4), SEQ ID NO: 38 (CHIKV DVG-IH1), SEQ ID NO: 39 (CHIKV DVG-IU1), SEQ ID NO: 40 (CHIKV DVG-CV1), SEQ ID NO: 41 (CHIKV DVG-CV2), SEQ ID NO: 42 (CHIKV DVG-CV3), SEQ ID NO: 43 (CHIKV DVG-CV4), SEQ ID NO: 44 (CHIKV DVG-CH1), SEQ ID NO: 45 (CHIKV DVG-CH2), SEQ ID NO: 46 (CHIKV DVG-CH3), SEQ ID NO: 47 (CHIKV DVG-CM1), SEQ ID NO: 48 (CHIKV DVG-CU1), SEQ ID NO: 49 (CHIKV DVG-CU2), SEQ ID NO: 50 (CHIKV DVG-CA1), SEQ ID NO: 51 (CHIKV DVG-CA2), SEQ ID NO: 52 (CHIKV DVG-CA3), SEQ ID NO: 53 (CHIKV DVG-CA4), SEQ ID NO: 55 (RV DVG A01a-TIP-01), SEQ ID NO: 56 (RV DVG A01a-TIP-02), SEQ ID NO: 57 (RVDVG A01a-TIP-03), SEQ ID NO: 58 (RV DVG A01a-TIP-04), SEQ ID NO: 59 (RVDVG A01a-TIP-05), SEQ ID NO: 60 (RV DVG A01a-TIP-06), SEQ ID NO: 62 (RVDVG A16-TIP-01), SEQ ID NO: 63 (RV DVG A16-TIP-02), SEQ ID NO: 64 (RV DVG A16-TIP-03), SEQ ID NO: 65 (RV DVG A16-TIP-04), SEQ ID NO: 66 (RV DVG A16-TIP-05), SEQ ID NO: 89 (RV DVG C15-TIP-01), SEQ ID NO: 90 (RV DVG C15-TIP-02), SEQ ID NO: 91 (RV DVG C15-TIP-03), SEQ ID NO: 92 (RV DVG C15-TIP-04), SEQ ID NO: 68 (RV DVG B14-TIP-01), SEQ ID NO: 69 (RV DVG B14-TIP-02), SEQ ID NO: 70 (RV DVG B14-TIP-03), SEQ ID NO: 71 (RV DVG B14-TIP-04), SEQ ID NO: 72 (RV DVG B14-TIP-05), SEQ ID NO: 73 (RV DVG B14-TIP-06), SEQ ID NO: 74 (RV DVG B14-TIP-07), SEQ ID NO: 75 (RV DVG B14-TIP-08), SEQ ID NO: 76 (RV DVG B14-TIP-09), SEQ ID NO: 77 (RV DVG B14-TIP-10), SEQ ID NO: 78 (RV DVG B14-TIP-11), SEQ ID NO: 79 (RV DVG B14-TIP-12), SEQ ID NO: 80 (RV DVG B14-TIP-13), SEQ ID NO: 81 (RV DVG B14-TIP-14), SEQ ID NO: 82 (RV DVG B14-TIP-15), SEQ ID NO: 83 (RV DVG B14-TIP-16), SEQ ID NO: 84 (RV DVG B14-TIP-17), SEQ ID NO: 85 (RV DVG B14-TIP-18), SEQ ID NO: 86 (RV DVG B14-TIP-19), SEQ ID NO: 87 (RV DVG B14-TIP-20), SEQ ID NO:93 (YFV DVG-A), SEQ ID NO: 94 (YFV DVG-B), SEQ ID NO: 95 (YFV DVG-C), SEQ ID NO: 96 (YFV DVG-D), SEQ ID NO: 97 (YFV DVG-E), SEQ ID NO: 98 (YFV DVG-F), SEQ ID NO: 99 (YFV DVG-G), SEQ ID NO: 100 (YFV DVG-H), SEQ ID NO: 101 (YFV DVG-I), SEQ ID NO: 102 (YFV DVG-J), SEQ ID NO: 103 (YFV DVG-K), SEQ ID NO: 104 (YFV DVG-L), SEQ ID NO:105 (WNV DVG-1), SEQ ID NO: 106 (WNV DVG-2), SEQ ID NO: 107 (WNV DVG-3), SEQ ID NO: 108 (WNV DVG-4), SEQ ID NO: 109 (WNV DVG-5), SEQ ID NO: 110 (WNV DVG-6), SEQ ID NO: 111 (SARS-CoV-2 DVG-1), SEQ ID NO: 112 (SARS-CoV-2 DVG_2) and SEQ ID NO: 113 (SARS-CoV-2 DVG_3), or a nucleotide sequence having at least 70% identity, more preferably at least 80% identity ; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences.

14. A defective interfering particle comprising the DVG of claim 12 or claim 13.

15. A method of treating a viral infection in a subject, comprising administering an efficient therapeutic amount of at least one DVG or defective interfering particle according to any one of claims 12 to 14.

16. The method according to claim 15, wherein the DVG is administered as a naked RNA.

17. The method according to claim 15, wherein at least one DVG is a defective interfering CHIKV genome.

18. The method according to claim 17, wherein at least one defective interfering CHIKV genome comprises or consists of a nucleotide sequence selected from the group consisting of: SEQ ID NO: 34 (CHIKV DVG-IV1), SEQ ID NO: 35 (CHIKV DVG-IV2), SEQ ID NO: 36 (CHIKV DVG-IV3), SEQ ID NO: 37 (CHIKV DVG-IV4), SEQ ID NO: 38 (CHIKV DVG-IH1), SEQ ID NO: 39 (CHIKV DVG-IU1), SEQ ID NO: 40 (CHIKV DVG-CV1), SEQ ID NO: 41 (CHIKV DVG-CV2), SEQ ID NO: 42 (CHIKV DVG-CV3), SEQ ID NO: 43 (CHIKV DVG-CV4), SEQ ID NO: 44 (CHIKV DVG-CH1), SEQ ID NO: 45 (CHIKV DVG-CH2), SEQ ID NO: 46 (CHIKV DVG-CH3), SEQ ID NO: 47 (CHIKV DVG-CM1), SEQ ID NO: 48 (CHIKV DVG-CU1), SEQ ID NO: 49 (CHIKV DVG-CU2), SEQ ID NO: 50 (CHIKV DVG-CA1), SEQ ID NO: 51 (CHIKV DVG-CA2), SEQ ID NO: 52 (CHIKV DVG-CA3) and SEQ ID NO: 53 (CHIKV DVG-CA4).

19. The method according to claim 15, wherein at least one defective interfering genome is a defective interfering ZIKV genome.

20. The method according to claim 19, wherein at least one interfering ZIKV genome comprises or consists of a nucleotide sequence selected from the group consisting of: SEQ ID NO: 22 (ZIKV DVG-A), SEQ ID NO: 23 (ZIKV DVG-B), SEQ ID NO: 24 (ZIKV DVG-C), SEQ ID NO: 25 (ZIKV DVG-D), SEQ ID NO: 26 (ZIKV DVG-E), SEQ ID NO: 27 (ZIKV DVG-F), SEQ ID NO: 28 (ZIKV DVG-G), SEQ ID NO: 29 (ZIKV DVG-H), SEQ ID NO: 30 (ZIKV DVG-I), SEQ ID NO: 31 (ZIKV DVG-J), SEQ ID NO: 32 (ZIKV DVG-K) and SEQ ID NO: 33 (ZIKV DVG-L), in particular SEQ ID NO: 32 (ZIKV DVG-K).

21. The method according to claim 15, wherein at least one defective interfering genome is a defective interfering EV71 genome.

22. The method according to claim 21, wherein at least one defective interfering EV71 genome comprises or consists of a nucleotide sequence selected from the group consisting of: SEQ ID NO: 1 (DG1 : EV71 DVG 293-390), SEQ ID NO: 2 (DG2 : EV71 DVG 294-404), SEQ ID NO: 3 (DG3 : EV71 DVG 1746-2895), SEQ ID NO: 4 (DG4 : EV71 DVG 1752-2894), SEQ ID NO: 5 (DG5 : EV71 DVG 1752-2896), SEQ ID NO: 6 (DG6 : EV71 DVG 1880-6487), SEQ ID NO: 7 (DG7 : EV71 DVG 1880-6488), SEQ ID NO: 8 (DG8 : EV71 DVG 3513-6516), SEQ ID NO: 9 (DG9 : EV71 DVG 5475-5634), SEQ ID NO: 10 (DG10: EV71 DVG 5610-6876), SEQ ID NO: 11 (DG11 : EV71 DVG 5610-6877), SEQ ID NO: 12 (DG12: EV71 DVG 5746-5821), SEQ ID NO: 13 (DG13 : EV71 DVG 6322-6356), SEQ ID NO: 14 (DG14: EV71 DVG 6322-6358), SEQ ID NO: 15 (DG15: EV71 DVG 6728-6779), SEQ ID NO: 16 (DG16: EV71 DVG 6966-7012), SEQ ID NO: 17 (DG17: EV71 DVG 6966-7014), SEQ ID NO: 18 (DG18: EV71 DVG 7098-7122), SEQ ID NO: 19 (DG19: EV71 DVG 7165-7200), SEQ ID NO: 20 (DG20: EV71 DVG 7165-7201) and SEQ ID NO: 21 (DG21: EV71 DVG 7238-7292).

23. The method according to claim 15, wherein at least one defective interfering genome is a defective interfering RV genome.

24. The method according to claim 23, wherein at least one defective interfering RV genome comprises or consists of a nucleotide sequence selected from the group consisting of: SEQ ID NO: 55 (RV DVG A01a-TIP-01), SEQ ID NO: 56 (RV DVG A01a-TIP-02), SEQ ID NO: 57 (RV DVG A01a-TIP-03), SEQ ID NO: 58 (RV DVG A01a-TIP-04), SEQ ID NO: 59 (RV DVG A01a-TIP-05), SEQ ID NO: 60 (RV DVG A01a-TIP-06), SEQ ID NO: 62 (RV DVG A16-TIP-01), SEQ ID NO: 63 (RV DVG A16-TIP-02), SEQ ID NO: 64 (RV DVG A16-TIP-03), SEQ ID NO: 65 (RV DVG A16-TIP-04), SEQ ID NO: 66 (RV DVG A16-TIP-05), SEQ ID NO: 89 (RV DVG C15-TIP-01), SEQ ID NO: 90 (RV DVG C15-TIP-02), SEQ ID NO: 91 (RV DVG C15-TIP-03), SEQ ID NO: 92 (RV DVG C15-TIP-04), SEQ ID NO: 68 (RV DVG B14-TIP-01), SEQ ID NO: 69 (RV DVG B14-TIP-02), SEQ ID NO: 70 (RV DVG B14-TIP-03), SEQ ID NO: 71 (RV DVG B14-TIP-04), SEQ ID NO: 72 (RV DVG B14-TIP-05), SEQ ID NO: 73 (RV DVG B14-TIP-06), SEQ ID NO: 74 (RV DVG B14-TIP-07), SEQ ID NO: 75 (RV DVG B14-TIP-08), SEQ ID NO: 76 (RV DVG B14-TIP-09), SEQ ID NO: 77 (RV DVG B14-TIP-10), SEQ ID NO: 78 (RV DVG B14-TIP-11), SEQ ID NO: 79 (RV DVG B14-TIP-12), SEQ ID NO: 80 (RV DVG B14-TIP-13), SEQ ID NO: 81 (RV DVG B14-TIP-14), SEQ ID NO: 82 (RV DVG B14-TIP-15), SEQ ID NO: 83 (RV DVG B14-TIP-16), SEQ ID NO: 84 (RV DVG B14-TIP-17), SEQ ID NO: 85 (RV DVG B14-TIP-18), SEQ ID NO: 86 (RV DVG B14-TIP-19) and SEQ ID NO: 87 (RV DVG B14-TIP-20).

25. The method according to claim 15, wherein the at least one defective interfering genome is a defective interfering YFV genome.

26. The method according to claim 25, wherein the at least one defective interfering YFV genome comprises or consists of a nucleotide sequence selected from the group consisting of: SEQ ID NO: 93 (YFV DVG-A), SEQ ID NO: 94 (YFV DVG-B), SEQ ID NO: 95 (YFV DVG-C), SEQ ID NO: 96 (YFV DVG-D), SEQ ID NO: 97 (YFV DVG-E), SEQ ID NO: 98 (YFV DVG-F), SEQ ID NO: 99 (YFV DVG-G), SEQ ID NO: 100 (YFV DVG-H), SEQ ID NO: 101 (YFV DVG-I), SEQ ID NO: 102 (YFV DVG-J), SEQ ID NO: 103 (YFV DVG-K) and SEQ ID NO: 104 (YFV DVG-L).

27. The method according to claim 15, wherein the at least one defective interfering genome is a defective interfering WNV genome.

28. The method according to claim 27, wherein the at least one defective interfering WNV genome comprises or consists of a nucleotide sequence selected from the group consisting of: SEQ ID NO:105 (WNV DVG-1), SEQ ID NO: 106 (WNV DVG-2), SEQ ID NO: 107 (WNV DVG-3), SEQ ID NO: 108 (WNV DVG-4), SEQ ID NO: 109 (WNV DVG-5) and SEQ ID NO: 110 (WNV DVG-6).

29. The method according to claim 15, wherein the at least one defective interfering genome is a defective interfering CV genome.

30. The method according to claim 29, wherein the at least one defective interfering CV genome comprises or consists of a nucleotide sequence selected from the group consisting of: SEQ ID NO: 111 (SARS-CoV-2 DVG_1), SEQ ID NO: 112 (SARS-CoV-2 DVG_2) and SEQ ID NO: 113 (SARS-CoV-2 DVG_3).

31. The defective interfering genome DVG or defective interfering particle according to any one of claims 12 to 14 for use in a method of treatment of CHIKV infection to a subject, in particular wherein the defective interfering genome comprises or consists of a nucleotide sequence selected from the group consisting of: SEQ ID NO: 34 (CHIKV DVG-IV1), SEQ ID NO: 35 (CHIKV DVG-IV2), SEQ ID NO: 36 (CHIKV DVG-IV3), SEQ ID NO: 37 (CHIKV DVG-IV4), SEQ ID NO: 38 (CHIKV DVG-IH1), SEQ ID NO: 39 (CHIKV DVG-IU1), SEQ ID NO: 40 (CHIKV DVG-CV1), SEQ ID NO: 41 (CHIKV DVG-CV2), SEQ ID NO: 42 (CHIKV DVG-CV3), SEQ ID NO: 43 (CHIKV DVG-CV4), SEQ ID NO: 44 (CHIKV DVG-CH1), SEQ ID NO: 45 (CHIKV DVG-CH2), SEQ ID NO: 46 (CHIKV DVG-CH3), SEQ ID NO: 47 (CHIKV DVG-CM1), SEQ ID NO: 48 (CHIKV DVG-CU1), SEQ ID NO: 49 (CHIKV DVG-CU2), SEQ ID NO: 50 (CHIKV DVG-CA1), SEQ ID NO: 51 (CHIKV DVG-CA2), SEQ ID NO: 52 (CHIKV DVG-CA3) and SEQ ID NO: 53 (CHIKV DVG-CA4).

32. The defective interfering genome DVG or defective interfering particle according to any one of claims 12 to 14 for use in a method of treatment of ZIKV infection to a subject, in particular wherein the defective interfering genome comprises or consists of a nucleotide sequence selected from the group consisting of: SEQ ID NO: 22 (ZIKV DVG-A), SEQ ID NO: 23 (ZIKV DVG-B), SEQ ID NO: 24 (ZIKV DVG-C), SEQ ID NO: 25 (ZIKV DVG-D), SEQ ID NO: 26 (ZIKV DVG-E), SEQ ID NO: 27 (ZIKV DVG-F), SEQ ID NO: 28 (ZIKV DVG-G), SEQ ID NO: 29 (ZIKV DVG-H), SEQ ID NO: 30 (ZIKV DVG-I), SEQ ID NO: 31 (ZIKV DVG-J), SEQ ID NO: 32 (ZIKV DVG-K) and SEQ ID NO: 33 (ZIKV DVG-L).

33. The defective interfering genome DVG or defective interfering particle according to any one of claims 12 to 14 for use in a method of treatment of EV71 infection to a subject, in particular wherein the defective interfering genome comprises or consists of a nucleotide sequence selected from the group consisting of: SEQ ID NO: 1 (DG1 : EV71 DVG 293-390), SEQ ID NO: 2 (DG2 : EV71 DVG 294-404), SEQ ID NO: 3 (DG3 : EV71 DVG 1746-2895), SEQ ID NO: 4 (DG4 : EV71 DVG 1752-2894), SEQ ID NO: 5 (DG5 : EV71 DVG 1752-2896), SEQ ID NO: 6 (DG6 : EV71 DVG 1880-6487), SEQ ID NO: 7 (DG7 : EV71 DVG 1880-6488), SEQ ID NO: 8 (DG8 : EV71 DVG 3513-6516), SEQ ID NO: 9 (DG9 : EV71 DVG 5475-5634), SEQ ID NO: 10 (DG10: EV71 DVG 5610-6876), SEQ ID NO: 11 (DG11 : EV71 DVG 5610-6877), SEQ ID NO: 12 (DG12: EV71 DVG 5746-5821), SEQ ID NO: 13 (DG13 : EV71 DVG 6322-6356), SEQ ID NO: 14 (DG14: EV71 DVG 6322-6358), SEQ ID NO: 15 (DG15: EV71 DVG 6728-6779), SEQ ID NO: 16 (DG16: EV71 DVG 6966-7012), SEQ ID NO: 17 (DG17: EV71 DVG 6966-7014), SEQ ID NO: 18 (DG18: EV71 DVG 7098-7122), SEQ ID NO: 19 (DG19: EV71 DVG 7165-7200), SEQ ID NO: 20 (DG20: EV71 DVG 7165-7201) and SEQ ID NO: 21 (DG21: EV71 DVG 7238-7292).

34. The defective interfering genome DVG or defective interfering particle according to any one of claims 12 to 14 for use in a method of treatment of RV infection to a subject, in particular wherein the defective interfering genome comprises or consists of a nucleotide sequence selected from the group consisting of: SEQ ID NO: 55 (RV DVG A01a-TIP-01), SEQ ID NO: 56 (RV DVG A01a-TIP-02), SEQ ID NO: 57 (RV DVG A01a-TIP-03), SEQ ID NO: 58 (RV DVG A01a-TIP-04), SEQ ID NO: 59 (RV DVG A01a-TIP-05), SEQ ID NO: 60 (RV DVG A01a-TIP-06), SEQ ID NO: 62 (RV DVG A16-TIP-01), SEQ ID NO: 63 (RV DVG A16-TIP-02), SEQ ID NO: 64 (RV DVG A16-TIP-03), SEQ ID NO: 65 (RV DVG A16-TIP-04), SEQ ID NO: 66 (RV DVG A16-TIP-05), SEQ ID NO: 89 (RV DVG C15-TIP-01), SEQ ID NO: 90 (RV DVG C15-TIP-02), SEQ ID NO: 91 (RV DVG C15-TIP-03), SEQ ID NO: 92 (RV DVG C15-TIP-04), SEQ ID NO: 68 (RV DVG B14-TIP-01), SEQ ID NO: 69 (RV DVG B14-TIP-02), SEQ ID NO: 70 (RV DVG B14-TIP-03), SEQ ID NO: 71 (RV DVG B14-TIP-04), SEQ ID NO: 72 (RV DVG B14-TIP-05), SEQ ID NO: 73 (RV DVG B14-TIP-06), SEQ ID NO: 74 (RV DVG B14-TIP-07), SEQ ID NO: 75 (RV DVG B14-TIP-08), SEQ ID NO: 76 (RV DVG B14-TIP-09), SEQ ID NO: 77 (RV DVG B14-TIP-10), SEQ ID NO: 78 (RV DVG B14-TIP-11), SEQ ID NO: 79 (RV DVG B14-TIP-12), SEQ ID NO: 80 (RV DVG B14-TIP-13), SEQ ID NO: 81 (RVDVGB14-TIP-14), SEQ ID NO: 82 (RV DVG B14-TIP-15), SEQ ID NO: 83 (RV DVG B14-TIP-16), SEQ ID NO: 84 (RV DVG B14-TIP-17), SEQ ID NO: 85 (RV DVG B14-TIP-18), SEQ ID NO: 86 (RV DVG B14-TIP-19) and SEQ ID NO: 87 (RV DVG B14-TIP-20).

35. The defective interfering genome DVG or defective interfering particle according to any one of claims 12 to 14 for use in a method of treatment of YFV infection to a subject, in particular wherein the defective interfering genome comprises or consists of a nucleotide sequence selected from the group consisting of: SEQ ID NO: 93 (YFV DVG-A), SEQ ID NO: 94 (YFV DVG-B), SEQ ID NO: 95 (YFV DVG-C), SEQ ID NO: 96 (YFV DVG-D), SEQ ID NO: 97 (YFV DVG-E), SEQ ID NO: 98 (YFV DVG-F), SEQ ID NO: 99 (YFV DVG-G), SEQ ID NO: 100 (YFV DVG-H), SEQ ID NO: 101 (YFV DVG-I), SEQ ID NO: 102 (YFV DVG-J), SEQ ID NO: 103 (YFV DVG-K) and SEQ ID NO: 104 (YFV DVG-L).

36. The defective interfering genome DVG or defective interfering particle according to any one of claims 12 to 14 for use in a method of treatment of WNV infection to a subject, in particular wherein the defective interfering genome comprises or consists of a nucleotide sequence selected from the group consisting of: SEQ ID NO:105 (WNV DVG-1), SEQ ID NO: 106 (WNV DVG-2), SEQ ID NO: 107 (WNV DVG-3), SEQ ID NO: 108 (WNV DVG-4), SEQ ID NO: 109 (WNV DVG-5) and SEQ ID NO: 110 (WNV DVG-6).

37. The defective interfering genome DVG or defective interfering particle according to any one of claims 12 to 14 for use in a method of treatment of CV infection to a subject, in particular a SARS-CoV-2 infection to a subject, in particular wherein the defective interfering genome comprises or consists of a nucleotide sequence selected from the group consisting of: SEQ ID NO: 111 (SARS-CoV-2 DVG_1), SEQ ID NO: 112 (SARS-CoV-2 DVG_2) and SEQ ID NO: 113 (SARS-CoV-2 DVG_3).

38. A pharmaceutical, immunogenic, or therapeutic composition, comprising at least one DVG according to claim 12 or claim 13, and a pharmaceutically acceptable carrier.

39. A pharmaceutical, immunogenic, or therapeutic composition, comprising at least one defective interfering particle according to claim 14, and a pharmaceutically acceptable carrier.

40. A vaccine comprising the composition according to claim 38 or 39.

41. Use of a DVG according to claim 12 or 13, or a defective interfering particle according to claim 14, for the preparation of a drug for the treatment of a patient infected with a target virus of the DVG or defective interfering particle.

42. Use of a DVG according to claim 12 or 13, or a defective interfering particle according to claim 14, for the preparation of a drug for the treatment of a patient infected with CHIKV.

43. Use of a DVG according to claim 12 or 13, or a defective interfering particle according to claim 14, for the preparation of a drug for the treatment of a patient infected with ZIKV.

44. Use of a DVG according to claim 12 or 13, or a defective interfering particle according to claim 14, for the preparation of a drug for the treatment of a patient infected with EV71.

45. Use of a DVG according to claim 12 or 13, or a defective interfering particle according to claim 14, for the preparation of a drug for the treatment of a patient infected with RV.

46. Use of a DVG according to claim 12 or 13, or a defective interfering particle according to claim 14, for the preparation of a drug for the treatment of a patient infected with YFV.

47. Use of a DVG according to claim 12 or 13, or a defective interfering particle according to claim 14, for the preparation of a drug for the treatment of a patient infected with WNV.

48. Use of a DVG according to claim 12 or 13, or a defective interfering particle according to claim 14, for the preparation of a drug for the treatment of a patient infected with CV, in particular SARS-CoV-2.

49. A polynucleotide comprising or consisting of a nucleotide sequence selected from the group consisting of: SEQ ID NO: 1 (DG1 : EV71 DVG 293-390), SEQ ID NO: 2 (DG2 : EV71 DVG 294-404), SEQ ID NO: 3 (DG3 : EV71 DVG 1746-2895), SEQ ID NO: 4 (DG4 : EV71 DVG 1752-2894), SEQ ID NO: 5 (DG5 : EV71 DVG 1752-2896), SEQ ID NO: 6 (DG6 : EV71 DVG 1880-6487), SEQ ID NO: 7 (DG7 : EV71 DVG 1880-6488), SEQ ID NO: 8 (DG8 : EV71 DVG 3513-6516), SEQ ID NO: 9 (DG9 : EV71 DVG 5475-5634), SEQ ID NO: 10 (DG10: EV71 DVG 5610-6876), SEQ ID NO: 11 (DG11 : EV71 DVG 5610-6877), SEQ ID NO: 12 (DG12: EV71 DVG 5746-5821), SEQ ID NO: 13 (DG13 : EV71 DVG 6322-6356), SEQ ID NO: 14 (DG14: EV71 DVG 6322-6358), SEQ ID NO: 15 (DG15: EV71 DVG 6728-6779), SEQ ID NO: 16 (DG16: EV71 DVG 6966-7012), SEQ ID NO: 17 (DG17: EV71 DVG 6966-7014), SEQ ID NO: 18 (DG18: EV71 DVG 7098-7122), SEQ ID NO: 19 (DG19: EV71 DVG 7165-7200), SEQ ID NO: 20 (DG20: EV71 DVG 7165-7201), SEQ ID NO: 21 (DG21: EV71 DVG 7238-7292), SEQ ID NO: 22 (ZIKV DVG-A), SEQ ID NO: 23 (ZIKV DVG-B), SEQ ID NO: 24 (ZIKV DVG-C), SEQ ID NO: 25 (ZIKV DVG-D), SEQ ID NO: 26 (ZIKV DVG-E), SEQ ID NO: 27 (ZIKV DVG-F), SEQ ID NO: 28 (ZIKV DVG-G), SEQ ID NO: 29 (ZIKV DVG-H), SEQ ID NO: 30 (ZIKV DVG-I), SEQ ID NO: 31 (ZIKV DVG-J), SEQ ID NO: 32 (ZIKV DVG-K), SEQ ID NO: 33 (ZIKV DVG-L), SEQ ID NO: 34 (CHIKV DVG-IV1), SEQ ID NO: 35 (CHIKV DVG-IV2), SEQ ID NO: 36 (CHIKV DVG-IV3), SEQ ID NO: 37 (CHIKV DVG-IV4), SEQ ID NO: 38 (CHIKV DVG-IH1), SEQ ID NO: 39 (CHIKV DVG-IU1), SEQ ID NO: 40 (CHIKV DVG-CV1), SEQ ID NO: 41 (CHIKV DVG-CV2), SEQ ID NO: 42 (CHIKV DVG-CV3), SEQ ID NO: 43 (CHIKV DVG-CV4), SEQ ID NO: 44 (CHIKV DVG-CH1), SEQ ID NO: 45 (CHIKV DVG-CH2), SEQ ID NO: 46 (CHIKV DVG-CH3), SEQ ID NO: 47 (CHIKV DVG-CM1), SEQ ID NO: 48 (CHIKV DVG-CU1), SEQ ID NO: 49 (CHIKV DVG-CU2), SEQ ID NO: 50 (CHIKV DVG-CA1), SEQ ID NO: 51 (CHIKV DVG-CA2), SEQ ID NO: 52 (CHIKV DVG-CA3), SEQ ID NO: 53 (CHIKV DVG-CA4), SEQ ID NO: 55 (RV DVG A01a-TIP-01), SEQ ID NO: 56 (RV DVG A01a-TIP-02), SEQ ID NO: 57 (RV DVG A01a-TIP-03), SEQ ID NO: 58 (RV DVG A01a-TIP-04), SEQ ID NO: 59 (RV DVG A01a-TIP-05), SEQ ID NO: 60 (RV DVG A01a-TIP-06), SEQ ID NO: 62 (RV DVG A16-TIP-01), SEQ ID NO: 63 (RV DVG A16-TIP-02), SEQ ID NO: 64 (RV DVG A16-TIP-03), SEQ ID NO: 65 (RV DVG A16-TIP-04), SEQ ID NO: 66 (RV DVG A16-TIP-05), SEQ ID NO: 89 (RV DVG C15-TIP-01), SEQ ID NO: 90 (RV DVG C15-TIP-02), SEQ ID NO: 91 (RV DVG C15-TIP-03), SEQ ID NO: 92 (RV DVG C15-TIP-04), SEQ ID NO: 68 (RV DVG B14-TIP-01), SEQ ID NO: 69 (RV DVG B14-TIP-02), SEQ ID NO: 70 (RV DVG B14-TIP-03), SEQ ID NO: 71 (RV DVG B14-TIP-04), SEQ ID NO: 72 (RV DVG B14-TIP-05), SEQ ID NO: 73 (RV DVG B14-TIP-06), SEQ ID NO: 74 (RV DVG B14-TIP-07), SEQ ID NO: 75 (RV DVG B14-TIP-08), SEQ ID NO: 76 (RV DVG B14-TIP-09), SEQ ID NO: 77 (RV DVG B14-TIP-10), SEQ ID NO: 78 (RV DVG B14-TIP-11), SEQ ID NO: 79 (RV DVG B14-TIP-12), SEQ ID NO: 80 (RV DVG B14-TIP-13), SEQ ID NO: 81 (RV DVG B14-TIP-14), SEQ ID NO: 82 (RV DVG B14-TIP-15), SEQ ID NO: 83 (RV DVG B14-TIP-16), SEQ ID NO: 84 (RV DVG B14-TIP-17), SEQ ID NO: 85 (RV DVG B14-TIP-18), SEQ ID NO: 86 (RV DVG B14-TIP-19), SEQ ID NO: 87 (RV DVG B14-TIP-20), SEQ ID NO:93 (YFV DVG-A), SEQ ID NO: 94 (YFV DVG-B), SEQ ID NO: 95 (YFV DVG-C), SEQ ID NO: 96 (YFV DVG-D), SEQ ID NO: 97 (YFV DVG-E), SEQ ID NO: 98 (YFV DVG-F), SEQ ID NO: 99 (YFV DVG-G), SEQ ID NO: 100 (YFV DVG-H), SEQ ID NO: 101 (YFV DVG-I), SEQ ID NO: 102 (YFV DVG-J), SEQ ID NO: 103 (YFV DVG-K), SEQ ID NO: 104 (YFVDVG-L), SEQ ID NO:105 (WNV DVG-1), SEQ ID NO: 106 (WNV DVG-2), SEQ ID NO: 107 (WNV DVG-3), SEQ ID NO: 108 (WNV DVG-4), SEQ ID NO: 109 (WNV DVG-5), SEQ ID NO: 110 (WNV DVG-6), SEQ ID NO: 111 (SARS-CoV-2 DVG_1), SEQ ID NO: 112 (SARS-CoV-2 DVG_2) and SEQ ID NO: 113 (SARS-CoV-2 DVG_3), or a polynucleotide having at least 70% identity, more preferably at least 80% identity; more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, or more preferably at least 99% identity with one of these sequences..

50. An expression vector or a plasmid comprising the polynucleotide according to claim 49.

51. A cell line producing the DVG according to claim 12 or 13.
